(19) United States
(12) Patent Application Publication
(10) Pub. No.: US 2022/0363766 A1
(43) Pub. Date: Nov. 17, 2022

(54) COMPOSITIONS AND METHODS FOR TREATING CYTOTOXIC T CELL RESISTANT TUMORS

(71) Applicant: Dana-Farber Cancer Institute, Inc., Boston, MA (US)

(72) Inventors: Kai WUCHERPFENNIG, Brookline, MA (US); Lucas FERRARI DE ANDRADE, Alston, MA (US); Adrienne LUOMA, Boston, MA (US)

(21) Appl. No.: 17/767,035

(22) PCT Filed: Oct. 9, 2020
(86) PCT No.: PCT/US2020/055009

§ 371 Date: Apr. 6, 2022

Related U.S. Application Data
(60) Provisional application No. 62/912,826, filed on Oct. 9, 2019.

(51) Int. Cl. C07K 16/28; C12N 15/113; A61K 31/4045; A61K 38/17; A61K 35/17; A61P 35/00

(52) U.S. Cl. CPC C07K 16/2833; C12N 15/1138; A61K 31/4045; A61K 38/1774; A61K 35/17; A61P 35/00; C12N 2310/14; C07K 2317/76; A61K 39/00

(57) ABSTRACT

Embodiments described herein are directed to compositions and methods for treating tumors resistant to checkpoint blockade by activating NK cells.

This application claims priority from U.S. Provisional Application No. 62/912,826, filed Oct. 9, 2019, the entire contents of which are incorporated herein by reference.

### GOVERNMENT INTERESTS

This invention was made with government support under grant numbers CA173750, T32 CA207021, and R01 CA238039 awarded by The National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of embodiments described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD

Embodiments described herein are directed to compositions and methods for treating tumors resistant to checkpoint blockade by activating NK cells.

### BACKGROUND

Checkpoint blockade with antibodies targeting the programmed cell death protein 1 (PD-1) or cytotoxic T-lymphocyte-associated protein 4 (CTLA-4) inhibitory receptors on T cells can induce durable anti-tumor immunity even in patients with advanced cancer. However, many patients fail to benefit from these therapies due to primary or secondary resistance. Cytotoxic T cells play a central role in the efficacy of checkpoint blockade based on their ability to recognize tumor-derived peptides bound to major histocompatibility complex class I (MHC-I) proteins. Recognition of such MHC-I—peptide complexes by the T cell receptor (TCR) triggers the release of interferon-γ (IFNγ) by T cells which inhibits tumor cell proliferation and enhances expression of MHC-I proteins on both tumor and dendritic cells. Resistance to checkpoint blockade is therefore frequently mediated by loss of MHC-I expression by tumor cells, either by mutation or epigenetic silencing of key genes in the MHC-I (B2M, TAP1, TAP2 and other genes) or IFNγ (JAK1, JAK2) pathways. A low number or loss of neoantigens also diminishes tumor immunity mediated by cytotoxic T cells. There are currently no alternative immunotherapies for patients with solid tumors resistant to checkpoint blockade and cytotoxic T cells.

### SUMMARY

Aspects described herein comprise a method of treating, preventing, or alleviating a symptom of a cancer in a subject. In embodiments, treating cancer is indicated by stopping or reducing tumor growth and/or metastasis.

In embodiments, the cancer is resistant to cytotoxic T cells. In embodiments, the cancer is an MCH class I deficient cancer or a cancer resistant to IFN gamma. In embodiments, the cancer is resistant to immunotherapy, such as a cancer resistant to anti-PD1 and/or anti-PD-L1 antibodies.

Non-limiting examples of such cancers that can be treated by embodiments described herein comprise melanoma, lung cancer, renal cancer, bladder cancer, Hodgkin's lymphoma, breast cancer, stomach cancer, and pancreatic cancer.

In an embodiment, the method comprises administering to the subject a therapeutically effective amount of a composition comprising one or more activating agent(s) and a pharmaceutically acceptable carrier. For example, the activating agent(s) activates NK cells through the NKG2D and/or CD16 receptors, thereby causing lysis of one or more cancer cells in the subject, and wherein the cancer is resistant to cytotoxic T cells.

In embodiments, the activating agent comprises a polynucleotide, a polypeptide, a small molecule, a cytokine, or a combination thereof. For example, the activating agent comprises an antibody, such as a monoclonal antibody. For example, the antibody comprises an anti-MICA antibody, an anti-MICB antibody, or both. In embodiments, the antibody binds the alpha-3 domain of MICA/B. In embodiments, the antibody comprises one or more sequences of Table 1.

In embodiments, the activating agent(s) inhibits MICA/MICB shedding by the tumor, thereby increasing the density of NKG2D receptor ligands on tumor cells. For example, the anti-MICA/MICB antibody inhibits the shedding of MICA/MICB by the tumor.

Embodiments can further comprise a step of administering to the subject a therapeutically effective amount of a second composition comprising one or more additional therapeutic or prophylactic agent(s) and a pharmaceutically acceptable carrier. In embodiments, the one or more additional therapeutic or prophylactic agent(s) comprises a toxin, a radiolabel, radiotherapy, an siRNA, a small molecule, a peptide, an antibody, a genetically engineered cell, radiation, or a cytokine.

For example, the one or more additional therapeutic or prophylactic agent comprises an HDAC inhibitor, such as panobinostat.

For example, the one or more additional therapeutic or prophylactic agent comprises cytokine, such as IL2, IL15, IL12, or IL18.

For example, the one or more additional therapeutic or prophylactic agent comprises a small molecule. In embodiments, the small molecule comprises a proteasome inhibitor.

For example, the one or more additional therapeutic or prophylactic agent comprises an antibody, such as an anti-PD1 antibody, anti-PDL1 antibody and/or anti-CTLA-4 antibody.

For example, the one or more additional therapeutic or prophylactic agent comprises a genetically engineered cell, such as a CAR T cell.

Embodiments herein can further comprise a step of testing the cancer for a Jak1 mutation and/or a B2m mutation.

Aspects described herein are also drawn to a method of sensitizing a cancer cell in a subject to NK cells.

In embodiments, the method comprises the method comprises administering to the subject a therapeutically effective amount of a composition comprising one or more activating agent(s) and a pharmaceutically acceptable carrier. For example, the activating agent(s) activates NK cells, thereby causing lysis of one or more cancer cells in the subject, and wherein the cancer is resistant to cytotoxic T cells.

In embodiments, activation of the NKG2D receptor and/or CD16 receptor activates NK cells.

In embodiments, the activating agent(s) inhibits MICA/MICB shedding by the cancer cell, thereby activating the NKG2D and/or CD16 receptor. Thus, MICA/B on the surface of the cancer cell activates the NKG2D receptor, the CD16 receptor, or both.

Other objects and advantages of embodiments described herein will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows the characterization of NK cells infiltrating human melanoma metastases by single-cell RNA-seq. (A) Isolation of NK cells from blood and melanoma metastasis (patient CY158) by flow cytometry. NK cells were identified as lymphocyte-size single viable cells that were positive for CD45 and CD56 markers but negative for CD3, CD4, CD8a, CD14, CD15 and CD163 markers. Numbers indicate the percentage of NK cells in the total lymphocyte population (which also includes T cells and B cells). (B) NK cells in tumor are different to NK cells in blood. A single cell RNA-seq analysis of blood and tumor-infiltrating NK cells for CY158 patient. UMAP plots were used to visualize blood and tumor-infiltrating NK cell populations and the percentage of NK cells in each cluster is indicated for blood and tumor NK cells (left). NK cell clusters are color coded and key differentially expressed genes are shown for each cluster (right). (C) Comparison of NK cell populations in metastases and blood by scRNA-seq, with data from three patients (CY155, CY158 and CY160) merged. NK cell clusters in blood (left) and metastases (right) were visualized using UMAP plots. Key differentially expressed genes are indicated for each cluster as well as the percentage of NK cells assigned to a given cluster. (D) mRNA transcripts for selected genes in blood (top) and tumor-infiltrating (bottom) NK cell populations were visualized using UMAP plots. The intensity of the blue color indicates the level of expression for indicated genes in individual cells.

FIG. 2 shows the identification of NK cell populations as well as expression of genes related to cytotoxicity and chemokines. (A-D) Identification of NK cell and ILC3 cell populations. Published single cell data from human innate lymphocytes isolated from tonsil were used to define gene expression signatures for NK cells and ILC3.³¹ UMAP plots and violin plots show the degree of similarity between these gene expression signatures and the gene expression patterns of sequenced cells. Cells from blood and tumors were interrogated using the NK cell signature (A, B) as well as the ILC3 signature (C, D). (E) Cytotoxicity gene expression signature (GZMA, GZMB, GZMH, GZMK, GZMM, PRF1, GNLY and NKG7) for NK cells isolated from blood (top) and melanoma metastases (bottom). UMAP and violin plots are shown to indicate the scores for this signature across NK cell clusters. (F, G) UMAP plots showing expression of chemokines XCL1 and XCL2 (abbreviated as XCL1/2) (F) as well as CCL3, CCL4, CCL4L2 and CCL5 (abbreviated as CCL3/4/4L2/5) (G) by blood (top) and tumor-infiltrating NK cells (bottom).

FIG. 3 shows differential expression of inhibitory receptors by NK cells in melanoma metastases compared to circulating NK cells and identification of NK cell populations by flow cytometry. (A) Expression of inhibitory receptors by NK cells isolated from blood and melanoma metastases. The intensity of the blue color indicates the level of expression for indicated genes in individual cells. (B) Expression of genes used for identification of NK cell populations (FGFBP2 and FCGR3A) and effector molecules (GZMA and GZMK). The intensity of the blue color indicates the level of expression for indicated genes in individual cells. (C) Validation of three NK cell populations identified by scRNA-seq in blood samples by flow cytometry using FGFBP2 and CD16a as markers. NK cells were identified by gating on CD45 and CD56 positive cells that were negative for CD3E, CD19, CD14, CD15, CD163 and a dead cell marker. A representative analysis is shown for CY165 patient. (D) Quantification of three NK cell populations in blood and tumor samples based on FGFBP2 and CD16a markers. Labeling for granzymes A and K is also shown for each of the three populations. MFI=Mean Fluorescence Intensity. Each dot in these graphs represents an individual patient. Statistical analysis was performed by two-way ANOVA, Bonferroni's post-hoc tests, *p<0.05, **p<0.01, ***p<0.001.

FIG. 4 shows B2M inactivation sensitizes human melanoma cells to MICA/B mAb. (A) Validation of efficiency of B2M gene inactivation. Human A375 melanoma cells were edited with control or B2M gRNAs (designated as Control and B2M-KO, respectively). Edited A375 cells were treated with the indicated concentrations of IFNγ for 24 hours and surface levels of HLA-A/B/C were quantified by flow cytometry. MFI=Mean Fluorescence Intensity. (B-C) Human A375 melanoma cells edited with control or B2M gRNAs were cultured for 24 hours with MICA (7C6-hIgG1) or isotype control antibodies at the indicated concentrations. Quantification of soluble MICA produced by edited melanoma cells using a sandwich ELISA (B). The 7C6 antibody does not interfere with detection of soluble MICA by ELISA, as reported previously.²⁰ MICA/B surface protein levels on control and B2M-edited melanoma cells were quantified by flow cytometry using PE-conjugated MICA/B mAb 6D4 (C). This mAb binds to the MICA/B α1-α2 domains and does not compete with the 7C6 antibody, as reported previously.²⁰ (D) Effect of human NK cells on A375 melanoma cells dependent on MHC-I expression and MICA mAb treatment. GFP⁺ A375 melanoma cells edited with control or B2M gRNAs were plated at a density of 5×10³ cells per well in a 96-well plate. Melanoma cells were pre-treated with 7C6-hIgG1 or isotype control mAbs (20 μg/ml) for 24 hours prior to addition of purified human NK cells at different effector to target ratios (0:1, 0.5:1 or 1:1). IL-2 (300 U/ml) was added to support NK cell survival. The number of GFP⁺ A375 melanoma cells was quantified by imaging cytometry using a Nexcelom Celigo instrument at multiple time points over 72-hours period. Data representative of three independent experiments (A-D). Statistical analyses were performed by two-way analysis of variance (ANOVA) with Bonferroni's multiple comparison test (D), *p<0.05, ***p<0.001.

FIG. 5 shows MICA/B mAb treatment induces immunity against metastases resistant to cytotoxic T cells. (A) B16F10-MICA cells edited with control, B2m or Jak1 gRNAs were treated for 24 hours with IFNγ (10 ng/ml) or solvent control (PBS), and surface levels of H-2K^{b} was analyzed by flow cytometry. (B) MICA mAb induced immunity against established metastases with inactivating mutations in B2m or Jak1 genes. B16F10-MICA melanoma cells were edited with control, B2m or Jak1 gRNAs, and 7×10⁵ tumor cells were injected i.v. into B cell deficient (Ighm^{-/-}) mice. On day 7, a subset of mice was euthanized for quantification of metastases, while the remaining mice were treated with 7C6-mIgG2a or control mAbs (200 μg i.p. on days 7, 8, and 12). On day 14, lung surface metastases were counted under a stereomicroscope. (C) Impact of MICA mAb treatment on survival of mice with B2m or Jak1 deficient melanoma metastases. WT mice were inoculated i.v. with 2×10⁵ B16F10-MICA cells edited with control, B2m or Jak1 gRNAs. Mice received 7C6-mIgG2a or isotype control mAbs on days 1 and 2 and mouse survival was recorded. (D) Expression of MHC-I by LLC1-MICA cells. LLC1-MICA cells were edited with control or B2m gRNAs, and were either stimulated with IFNγ (10 ng/ml) or solvent control (PBS) for 24 hours. Surface H-2K^{b} protein levels were quantified by flow cytometry. (E) MICA mAb treatment of lung metastases formed by LLC1 lung cancer cells. WT C57BL6/J mice were inoculated i.v. with 1×10⁶ (1M) or 1.5×10⁶ (1.5M) LLC1-MICA tumor cells edited with control or B2m gRNAs. On day 2 following tumor cell inoculation, mice were treated with indicated mAb (200 μg i.p.); additional treatments were given on day 3 and then once per week. Lung metastases were counted on day 14. (F) MICA mAb treatment of LLC1-MICA metastases in mice reconstituted with allogeneic or syngeneic NK cells. Rag2^{-/-}Il2rg^{-/-} double knockout mice were injected with NK cells (2×10⁵ cells) from CB6F1/J mice or C56BL/6 mice, which were allogeneic or syngeneic to LLC1 cells, respectively. A third group of Rag2^{-/-} Il2rg^{-/-} mice did not receive NK cells. LLC1-MICA tumor cells (7×10⁵) were injected i.v. 24 hours following NK cell transfer. On days 2, 3, and then once per week following tumor cell inoculation, mice were treated with the indicated antibodies (200 μg). Metastases were counted on day 14. Data representative of three independent experiments (A, D) or pooled from three (B, E) or two (C, F) independent experiments. Statistical analyses were performed by two-tailed unpaired Student's t tests (B, E-F), and Log-rank (Mantel-Cox) test (C). *p<0.05, **p<0.01, ***p<0.001.

FIG. 6 shows NK cells are essential for treatment of B2m and Jak1 deficient melanoma metastases with a MICA/B antibody. (A) Wild-type (WT) C57BL/6 mice were inoculated i.v. with 7×10⁵ B16F10-MICA cells that had been edited with control, B2m or Jak1 gRNAs. Mice were treated with 7C6-mIgG2a or isotype control mAbs (200 μg) one day later as well as on days 2 and 7. NK cell depletion was performed by injection of 100 μg of anti-asialo GM1 (anti-asGM1) on days −1, 0, and 7 relative to tumor cell inoculation; control mice received an isotype control antibody. Lung surface metastases were quantified on day 14 following tumor inoculation. (B) Analysis of NK cell infiltration into lung tissue. Tumor injection and mAb treatment were done as described in ‘A’, with tumor cells that expressed ZsGreen that enabled identification by flow cytometry. On day 12 following tumor cell inoculation, mice were injected i.v. with an APC-conjugated anti-CD45.2 antibody to distinguish blood and tissue-infiltrating NK cells, as reported previously.²⁰ Lung-infiltrating NK cells were identified as CD3ϵ− TCRβ− NK1.1+ CD49b+ EOMES+ viable cells with low staining for CD45.2-APC (injected i.v.) but high staining for CD45.2-PE-CY7 (added to cell suspension). The ratio of NK cells to ZsGreen⁺ B16F10-MICA cells is shown. (C, D) Numbers of ZsGreen⁺ B16F10-MICA cells (C) and lung-infiltrating NK cells (D) for the indicated genotypes and treatment groups for the experiment described in (B). EOMES labeling was used to differentiate NK cells from ILC1. Data pooled from two independent experiments (A-D). Statistical analyses were performed using two-way ANOVA with Bonferroni's posthoc test (A) or two-tailed unpaired Student's t tests (B-D), *p<0.05, **p,0.01, ***p<0.001.

FIG. 7 shows the combination of the HDAC inhibitor panobinostat and a MICA mAb enhances surface levels of MICA/B and inhibits growth of metastases in NSG mice reconstituted with human NK cells. (A) Increase in NKG2D ligand mRNA levels following treatment with panobinostat. A375 melanoma cells were treated for 24 hours with panobinostat (50 nM), and mRNA was extracted for bulk RNA-seq. mRNA levels for NKG2D ligand and MHC class I genes are shown as ratio (log 2 fold change) for the panobinostat and PBS groups. (B) Increase in MICA/B surface protein levels following treatment with panobinostat plus MICA/B mAb. A375 melanoma cells were incubated with the indicated mAbs (20 μg/ml) and increasing concentrations of panobinostat for 24 hours. MICA/B surface levels (left) and A375 cell viability (right) were quantified by flow cytometry. Shed MICA was quantified by sandwich ELISA (middle). (C) Treatment of short-term human melanoma cell lines with the combination of panobinostat plus MICA/B mAb. The indicated melanoma cell lines were treated in vitro with the indicated mAbs (20 μg/ml) plus increasing concentrations of panobinostat for 24 hours. MICA/B surface levels were quantified by flow cytometry. (D) In vivo synergy of panobinostat plus MICA/B mAb treatment on MICA/B surface protein levels in metastases formed by human melanoma cells. NSG mice were inoculated i.v. with 1×10⁶ ZsGreen+ A375 melanoma cells. Two weeks later, mice were treated on two subsequent days with the indicated mAbs (200 μg)+/−panobinostat (10 mg/kg). 24 hours following the last treatment, MICA/B surface levels were analyzed on tumor cells in lung metastases (large, viable, ZsGreen+, CD45− cells). (E-F) NSG mice were reconstituted with purified human NK cells (2×10⁶ i.v.) that had been expanded in vitro. In vivo survival of NK cells was supported by simultaneous administration of IL-2 (7.5×10⁴ units) via intraperitoneal injection. On day 1, mice were inoculated i.v. with control or B2M edited A375 cells (5×10⁵). On days 2 and 3, mice received another dose of IL-2, the indicated mAbs (200 μg)+/−10 mg/kg panobinostat. On day 14, the number of lung surface metastases was counted. Illustration of experimental design (E) and quantification of lung surface metastases (F). Data representative of three independent experiments (B and C), or pooled from two independent experiments (D and F). Statistical analyses were performed by two-tailed unpaired Student's t test (D) and two-way ANOVA, Bonferroni's post-hoc tests (F), *p<0.05, **p<0.01, ***p<0.001.

FIG. 8 shows flow cytometry analysis of NK cells from patients' blood and tumor samples. NK cells were identified as lymphocyte-size viable cells that expressed CD45 and CD56, but that did not express CD3, CD19 (in some of the samples), CD14, CD15 and CD163. Percentage of NK cells in the total lymphocyte population (including T and B cells) for blood NK cells (left) and tumor-infiltrating NK cells (right).

FIG. 9 shows comparison of circulating and tumor-infiltrating NK cell populations from individual patients by scRNA-seq. (A-B) Single cell RNA-seq analysis of blood and tumor-infiltrating NK cells is shown for samples from two patients, CY155 (A) and CY160 (B). UMAP plots were used to visualize blood and tumor-infiltrating NK cell populations from each patient. Also, the percentage of NK cells in each cluster is indicated for blood and tumor NK cells (left). NK cell clusters are color coded and key differentially expressed genes are shown for each cluster (right).

FIG. 10 shows analysis of ILC1 and ILC2 gene expression signatures and expression of chemokines. (A-B) Published single cell data from human innate lymphocytes isolated from tonsil were used to define gene expression signatures for ILC1 cells and ILC2.³¹ These signatures were used to investigate blood (A) and tumor (B) NK cells. (C) Expression of chemokines by NK cells isolated from blood and melanoma metastases. The intensity of the blue color indicates the level of expression for indicated genes in individual cells.

FIG. 11 shows analyses of surface receptors and gene expression signatures. (A-B) Gene expression signatures for activating NK cell receptors (KLRK1, KLRF1, FCGR3A, CD226, CD244, NCR1, NCR2 and NCR3) and inhibitory NK cell receptors (KIR2DL1, KIR2DL2, KIR2DL3, KIR2DL4, KLRC1, TIGIT, CD96, HAVCR2, PDCD1, LAG3) were used to compare blood (A) and tumor-infiltrating (B) NK cells. These signatures were visualized using UMAP and violin plots. (C) Expression of activating and inhibitory receptors by single cells by blood (top) and tumor (bottom) NK cells. The intensity of the blue color indicates the level of expression for indicated genes in individual cells. (D) Expression of NKG2D by NK cells as analyzed by flow cytometry. The histograms for CY158 patient are shown for illustration.

FIG. 12 shows analysis of surface HLA class I and MICA/B proteins on tumor cells from melanoma metastases. Melanoma metastases were surgically resected from patients and analyzed by flow cytometry. Tumor cells were identified as viable large single cells that were CD45 negative. (A, B) Expression of classical MHC-I (HLA-A/B/C) (A) and MICA/B (B) proteins by melanoma cells from lesions for which NK cells were also analyzed by scRNA-seq. (C-D) Expression of classical MHC-I proteins (C) and MICA/B (D) by tumor cells in metastases from an additional group of patients. (E) Quantification of shed MICA in plasma from the indicated melanoma patients (CY156P-CY166) and healthy donors (HD) by sandwich ELISA. Shed MICB was not detected in these samples.

FIG. 13 shows characterization of B2M deficient A375 melanoma cells and inhibition of NK cell-mediated killing of melanoma cells by recognition of MHC-I. (A) Validation of efficiency of B2M gene inactivation in A375 melanoma cells. Cells were edited with control or B2M gRNAs (designated as Control and B2M-KO, respectively). Cells were treated with the indicated concentration of IFNγ (1 ng/ml) or PBS for 24 hours, and surface HLA-A/B/C proteins were quantified by flow cytometry. (B) Comparison of MICA/B surface levels on control and B2M edited A375 cells. Cells were treated with 7C6-hIgG1 or isotype control mAbs (10 μg/ml) for 24 hours and MICA/B surface protein was analyzed by flow cytometry. (C) NK cells isolated from a healthy donor were cultured for 24 hours in the presence of 1000 U/ml of IL-2. Parental A375 melanoma cells were incubated 7C6-hIgG1 or isotype control antibodies (20 μg/ml) for 24 hours prior to use in the cytotoxicity assay. NK cell-mediated killing of A375 cells was analyzed using a 4-hour ⁵¹Cr-release assay. Indicated KIR or isotype control antibodies were added to co-cultures at 10 μg/ml. Data are representative of three independent experiments (A-C). Statistical analysis was performed by two-way ANOVA, Bonferroni's post-hoc test (C), ***p<0.001.

FIG. 14 shows characterization of B16F10-MICA cell lines and in vivo activity of MICA mAb. (A) B16F10-MICA cells edited with control, B2m or Jak1 gRNAs were treated for 24 hours with the indicated concentrations of IFNγ, and surface levels of H-2K^{b} (left) and MICA (right) were analyzed by flow cytometry. MFI=Mean Fluorescence Intensity. Data representative of three independent experiments. (B) B cell deficient (Ighm^{-/-}) mice were inoculated i.v. with 7×10⁵ B16F10-MICA cells edited with control, B2m or Jak1 gRNAs. When metastases were established (day 7), mice were treated with MICA or isotype control mAbs (200 μg on days 7, 8 and 12). Shed MICA in plasma samples was quantified using a sandwich ELISA. Data were pooled from two independent experiments. Statistical analyses were done by two-way ANOVA, Bonferroni's post-hoc test, ***p<0.001.

FIG. 15 shows effect of panobinostat treatment on a panel of human tumor cell lines. (A-B) A375 melanoma cells were treated with panobinostat (50 nM) or solvent control (PBS) for 24 hours, and gene expression was examined by bulk RNA-seq. Key differentially expressed genes in cells treated with panobinostat or solvent control (A) and key immunological pathways (B) upregulated in panobinostat compared to control treated A375 cells. FDR, false discovery rate; q-val, q value. (C-H) The indicated human tumor cell lines were treated with the indicated antibodies (20 μg/ml) and increasing concentrations of the HDAC inhibitor panobinostat for 24 hours. MICA/B surface levels were quantified by flow cytometry using PE-labeled 6D4 mAb (left graphs in C-H). Shed MICA in supernatants was quantified by sandwich ELISA (right graphs in C and D). (E-H) The ELISA kit did not detect shed MICA for these cell lines, likely due to specificity for allele variants or shedding of MICB (ELISA was specific for MICA). 7C6 antibodies with different Fc regions were used according to the availability of such antibodies at the time of the assays; the Fc region of this antibody does not affect the inhibition of MICA/B shedding, as previously reported.²⁰ Data representative of three independent experiments.

FIG. 16 shows panobinostat did not inhibit reconstitution of NSG mice with human NK cells. NSG mice were injected i.v. with 2×10⁶ in vitro-expanded human NK cells from healthy donors. Immediately following NK cell inoculation, mice were treated with IL-2 (7.5×10⁴ units) to support NK cell survival; mice also received panobinostat (10 mg/kg in PBS) or PBS as a control. 24 hours later, blood NK cells were analyzed by flow cytometry. (A) Number of circulating NK cells identified as CD45+ CD56+ CD3− viable cells. (B, C) Percentage of blood NK cells labeled with CD16a (B) or NKG2D (C) mAbs. Data pooled from two independent experiments (A-C).

FIG. 17 shows summary of investigated melanoma metastases. ScRNA-seq analysis was performed for the top three cases (highlighted in red); other tumor samples were used to examine tumor cell populations for expression of MHC class I and MICA/B proteins and NK cells by flow cytometry. The location of surgically resected metastases and prior treatment history are listed.

FIG. 18 shows inactivation of B2M gene enhances NK cell-mediated killing of human melanoma cells in the presence of a MICA/B mAb. (A) Validation of efficiency of B2M gene inactivation. Control or B2M-KO human A375 melanoma cells were treated with the indicated concentrations of IFNγ for 24 hours and surface levels of HLA-A/B/C were quantified by flow cytometry. MFI=Mean Fluorescence Intensity. (B-D) Control or B2M-KO human A375 melanoma cells were cultured for 24 hours with MICA/B (7C6-hIgG1) or isotype control antibodies at the indicated concentrations. Quantification of shed MICA released by melanoma cells using a sandwich ELISA (B). The 7C6 antibody did not interfere with detection of soluble MICA by ELISA, as reported previously (23). MICA/B surface protein levels on control and B2M-KO melanoma cells were quantified by flow cytometry using PE-conjugated MICA/B mAb 6D4 (C). This mAb binds to the MICA/B α1-α2 domains and does not compete with the 7C6 antibody, as reported previously (23). Histograms representative of the experiment shown in ‘C’ (D). (E) Effect of human NK cells on A375 melanoma cells dependent on MHC-I expression and MICA/B mAb treatment. GFP⁺ A375 melanoma cells (control or B2M-KO) were plated at a density of 5×10³ cells per well in a 96-well plate. Melanoma cells were pre-treated with 7C6-hIgG1 or isotype control mAbs (20 g/ml) for 24 hours prior to addition of purified human NK cells at different effector to target ratios (0:1, 0.5:1 or 1:1). IL-2 (300 U/ml) was added to support NK cell survival. The number of GFP⁺ A375 melanoma cells was quantified by imaging cytometry using a Nexcelom Celigo instrument at multiple time points over a 72-hour period. Data representative of three independent experiments (A-E). Statistical analyses were performed by two-way analysis of variance (ANOVA) with Bonferroni's multiple comparison test (E), *p<0.05, ***p<0.001.

FIG. 19 shows MICA/B mAb treatment induces immunity against melanoma metastases with inactivating mutations in B2m and Jak1 genes. (A) B16F10-MICA cells (control, B2m-KO or Jak1-KO) were treated for 24 hours with IFNγ (10 ng/ml) or solvent control (PBS), and surface level of H-2K^{b} was analyzed by flow cytometry. (B) MICA/B mAb treatment for established metastases with inactivating mutations in B2m or Jak1 genes. B16F10-MICA melanoma cells (7×10⁵ control, B2m-KO or Jak1-KO tumor cells) were injected i.v. into B cell deficient (Ighm^{-/-}) mice. On day 7, a subset of mice was euthanized for quantification of metastases, while the remaining mice were treated with 7C6-mIgG2a or control mAbs (200 μg i.p. on days 7, 8, and 12). On day 14, lung surface metastases were counted under a stereomicroscope. (C) Impact of MICA/B mAb treatment on survival of mice with B2m or Jak1 deficient melanoma metastases. WT mice (Ighm^{+/+}) were inoculated i.v. with 2×10⁵ control, B2m-KO or Jak1-KO B16F10-MICA cells. Mice received 7C6-mIgG2a or isotype control mAbs on days 1 and 2, and mouse survival was recorded. Data representative of three independent experiments (A) or pooled from three (B) or two (C) independent experiments. Statistical analyses were performed by two-tailed unpaired Student's t-tests (B), and Log-rank (Mantel-Cox) test (C). *p<0.05, **p<0.01, ***p<0.001.

FIG. 20 shows classical MHC-I molecules expressed by lung cancer cells inhibit NK cells and reduce the efficacy of MICA/B antibody treatment. (A) Expression of MHC-I by LLC1-MICA cells. Control or B2m-KO LLC1-MICA cells were either stimulated with IFNγ (10 ng/ml) or solvent control (PBS) for 24 hours. Surface H-2K^{b} protein levels were quantified by flow cytometry. (B) MICA/B mAb treatment of lung metastases formed by LLC1 lung cancer cells. WT C57BL6/J mice were inoculated i.v. with 1×10⁶ (1M) or 1.5×10⁶ (1.5M) LLC1-MICA tumor cells (control or B2m-KO). On day 2 following tumor cell inoculation, mice were treated with indicated mAb (200 μg i.p.); additional treatments were given on day 3 and then once per week. Lung metastases were counted on day 14. (C) MICA/B mAb treatment of LLC1-MICA metastases in mice reconstituted with allogeneic or syngeneic NK cells. Rag2^{-/-}Il2rg^{-/-} double knockout mice were injected with NK cells (2×10⁵ cells) from CB6F1/J mice or C56BL/6 mice, which were allogeneic or syngeneic to LLC1 cells, respectively. A third group of Rag2^{-/-}Il2rg^{-/-} mice did not receive NK cells. LLC1-MICA tumor cells (7×10⁵) were injected i.v. 24 hours following NK cell transfer. On days 2, 3, and then once per week following tumor cell inoculation, mice were treated with the indicated antibodies (200 μg). Metastases were counted on day 14. Data representative of three independent experiments (A) or pooled from three (B) or two (C) independent experiments. Statistical analyses were performed by two-tailed unpaired Student's t-test (B-C). *p<0.05, **p<0.01, ***p<0.001.

FIG. 21 shows NK cells are essential for treatment of B2m and Jak1 deficient melanoma metastases with a MICA/B antibody. (A) Wild-type (WT) C57BL/6 mice were inoculated i.v. with 7×10⁵ B16F10-MICA cells (control, B2m-KO or Jak1-KO). Mice were treated with 7C6-mIgG2a or isotype control mAbs (200 μg) one day later as well as on days 2 and 7. CD8 T cell depletion was performed by injection of 100 μg of anti-CD8β, whereas NK cell depletion was performed by injection of 100 g of anti-asialo GM1 (anti-asGM1) or anti-NK1.1; all depleting antibodies were administered on days −1, 0, and 7 relative to tumor cell inoculation; control mice received an isotype control antibody. Lung surface metastases were quantified on day 14 following tumor inoculation. (B) Analysis of NK cell infiltration into lung tissue. Tumor injection and mAb treatment were done as described in ‘A’, with tumor cells that expressed ZsGreen to enable their identification by flow cytometry. On day 12 following tumor cell inoculation, mice were injected i.v. with an APC-conjugated anti-CD45.2 antibody to distinguish blood and tissue-infiltrating NK cells, as reported previously (23). Lung-infiltrating NK cells were identified as CD3ε− TCRβ− NK1.1+ CD49b+ EOMES+ viable cells with low staining for CD45.2-APC (injected i.v.) but high staining for CD45.2-PE-CY7 (added to cell suspension). The ratio of NK cells to ZsGreen⁺ B16F10-MICA cells is shown. (C, D) Numbers of ZsGreen⁺ B16F10-MICA cells (C) and lung-infiltrating NK cells (D) for the indicated genotypes and treatment groups for the experiment described in (B). EOMES labeling was used to differentiate NK cells from ILC1. Data pooled from two independent experiments (A-D). Statistical analyses were performed using two-way ANOVA with Bonferroni's posthoc test (A) or two-tailed unpaired Student's t-test (B-D), *p<0.05, **p,0.01, ***p<0.001.

FIG. 22 shows the combination of the HDAC inhibitor panobinostat and a MICA/B mAb enhances surface levels of MICA/B on tumor cells. (A) Increase in NKG2D ligand mRNA levels following treatment with panobinostat. A375 melanoma cells were treated for 24 hours with panobinostat (50 nM), and mRNA was extracted for bulk RNA-seq. mRNA levels for NKG2D ligand and MHC class I genes are shown as ratio (log 2 fold change) for the panobinostat and PBS groups. (B) A375 melanoma cells were treated for 24 hours with panobinostat (50 nM) or solvent control (PBS), and the expression of the indicated genes was analyzed by RT-qPCR (triplicates per condition). *p<0.05, **p<0.01, and ***p<0.001, statistical analysis was performed using two-tailed unpaired Student's t-test with Welch's correction. Error bars represent standard deviation of three technical replicates. (C) Increase in MICA/B surface protein levels following treatment with panobinostat plus MICA/B mAb. A375 melanoma cells were incubated with the indicated mAbs (20 μg/ml) and increasing concentrations of panobinostat for 24 hours. MICA/B surface levels (left) and A375 cell viability (right) were quantified by flow cytometry. Shed MICA was quantified by sandwich ELISA (middle). (D) Representative histograms of the data shown in FIG. 22C. (E) Treatment of short-term human melanoma cell lines with the combination of panobinostat plus MICA/B mAb. The indicated melanoma cell lines were treated in vitro with the indicated mAbs (20 μg/ml) plus increasing concentrations of panobinostat for 24 hours. MICA/B surface levels were quantified by flow cytometry. Cell lines had different basal and induced levels of MICA/B; they were ordered from low to high MICA/B expression.

Data representative of three independent experiments (B, C, and E).

FIG. 23 shows HDAC-MICA/B antibody combination therapy inhibits growth of metastases in NSG mice reconstituted with human NK cells. (A) In vivo synergy of panobinostat plus MICA/B mAb treatment on MICA/B surface protein levels in metastases formed by human melanoma cells. NSG mice were inoculated i.v. with 1×10⁶ ZsGreen+A375 melanoma cells. Two weeks later, mice were treated on two subsequent days with the indicated mAbs (200 μg)+/−panobinostat (10 mg/kg). 24 hours following the last treatment, MICA/B surface levels were analyzed on tumor cells in lung metastases (large, viable, ZsGreen+, CD45− cells). (B-C) NSG mice were reconstituted with purified human NK cells (2×10⁶ i.v.) that had been expanded in vitro. In vivo survival of NK cells was supported by simultaneous administration of IL-2 (7.5×10⁴ units) via intraperitoneal injection. On day 1, mice were inoculated i.v. with control or B2M-KO A375 cells (5×10⁵). On days 2 and 3, mice received another dose of IL-2, the indicated mAbs (200 μg)+/−10 mg/kg panobinostat; on day 3 an additional dose of NK cells was also administered. On day 14, the number of lung surface metastases was counted. Illustration of experimental design (B) and quantification of lung surface metastases (C). Data pooled from two independent experiments (A and C). Statistical analyses were performed by two-tailed unpaired Student's t-test (A) and two-way ANOVA, Bonferroni's post-hoc test (C), *p<0.05, **p<0.01, ***p<0.001.

FIG. 24 shows characterization of B2M deficient A375 melanoma cells and inhibition of NK cell-mediated killing of melanoma cells by recognition of MHC-1. (A) Validation of efficiency of B2M gene inactivation in A375 melanoma cells. Control and B2M-KO cells were treated with the indicated concentration of IFNγ (1 ng/ml) or PBS for 24 hours, and surface HLA-A/B/C protein levels were quantified by flow cytometry. (B) Control and B2M-KO A375 melanoma cells were treated with or without IFNγ (50 ng/ml) for 24 hours. Western blots (20 g of total protein per lane) were probed with antibodies specific for B2M and tubulin. (C) NK cells isolated from a healthy donor were cultured for 24 hours in the presence of 1,000 U/ml of IL-2. Parental A375 melanoma cells were treated with 7C6-hIgG1 or isotype control antibodies (20 μg/ml) for 24 hours prior to use in the cytotoxicity assay. NK cell-mediated killing of A375 cells was analyzed using a 4-hour ⁵¹Cr-release assay. Indicated KIR or isotype control antibodies were added to co-cultures at 10 μg/ml. Data are representative of three independent experiments (A-C). Statistical analysis was performed by two-way ANOVA, Bonferroni's post-hoc test (C), ***p<0.001.

FIG. 25 shows characterization of B16F10-MICA cell lines and in vivo activity of MICA/B mAb. Control, B2m-KO or Jak1-KO B16F10-MICA cells were treated for 24 hours with the indicated concentrations of IFNγ. (B) Surface levels of H2-D^{b} were analyzed by flow cytometry. MFI=Mean Fluorescence Intensity. (C) Control, B2M-KO or Jak1-KO B16F10 melanoma cells were treated with or without IFNγ (50 ng/ml) for 24 hours. Western blots (20 g of total protein per lane) were probed with antibodies specific for B2M, JAK1 or GAPDH (loading control). Data representative of three independent experiments.

FIG. 26 shows B2m-KO and Jak1-KO B16F10-MICA cells are resistant to CD8 T cell-mediated cytotoxicity. Control, B2m-KO and Jak1-KO B16F10 melanoma cells were pulsed overnight with Ova peptide (10 nM), washed and added to 96 well plates (5,000 cells per well). Naïve OT-I T cells were added at different effector to target ratios (1:1, 2:1 and 5:1; the 0:1 condition contained no T cells). Cells were co-cultured for 48 hours (8-10 replicates per condition); wells were then washed to remove T cells as well as dead tumor cells, and adherent live tumor cells were counted using a Celigo Image Cytometer. Statistical significance was determined using a multiple t-test, error bars represent the standard deviation, *** p<0.0001.

FIG. 27 shows characterization of control and B2m-KO LLC1-MICA cell lines. (A) Control and B2m-KO LLC1-MICA cell lines were cultured for 24 hours with the indicated concentrations of IFNγ, and surface expression of H2-D^{b} was analyzed by flow cytometry. (B) Control or B2M-KO LLC1-MICA cells were treated with or without IFNγ (50 ng/ml) for 24 hours. Western blots (20 g of total protein per lane) were probed with antibodies specific for B2M or tubulin (loading control).

FIG. 28 shows in vivo efficacy of 7C6 antibody in the B16F10 metastasis model. (A) B cell deficient (Ighm^{-/-}) mice were inoculated i.v. with 7×10⁵ control, B2m-KO or Jak1-KO B16F10-MICA cells. When metastases were established (day 7), mice were treated with MICA/B or isotype control mAbs (200 μg on days 7, 8 and 12). Shed MICA in plasma samples was quantified using a sandwich ELISA. Data were pooled from two independent experiments. Statistical analyses were performed by two-way ANOVA, Bonferroni's post-hoc test, ***p<0.001. (B) WT mice were inoculated intravenously with control, B2m-KO or Jak1-KO B16F10-MICA cells and treated with the indicated antibodies as described in FIGS. 21A and B. Surface expression of NKG2D and CD16 receptors was analyzed on lung-infiltrating NK cells by flow cytometry. Data were pooled from two independent experiments. *p<0.05, calculated with two-tailed unpaired Student's t-test.

FIG. 29 shows the effect of panobinostat on gene expression by A375 cells. (A-B) A375 cells were treated for 24 hours with panobinostat (50 nM) or PBS and analyzed by bulk RNA-seq, as described in FIG. 22A. Key differentially expressed genes in cells treated with panobinostat or solvent control (A) and key immunological pathways (B) upregulated in panobinostat compared to control treated A375 cells. FDR, false discovery rate; q-val, q-value.

FIG. 30 shows the effect of panobinostat treatment on MICA/B expression by melanoma cells. Representative histograms for data on primary melanoma cell lines shown in FIG. 22E.

FIG. 31 shows the effect of panobinostat and 7C6 antibody on MICA/B expression by a diverse panel of tumor cell lines. (A-F) The human tumor cell lines were treated with the indicated antibodies (20 μg/ml) and increasing concentrations of the HDAC inhibitor panobinostat for 24 hours. MICA/B surface levels were quantified by flow cytometry using PE-labeled 6D4 mAb (left graphs in A, B and graphs C-F). Shed MICA in supernatants was quantified by sandwich ELISA (right graphs in A and B). 7C6 antibodies with different Fc regions were used based on the availability of such antibodies at the time of the assays; the Fc region of this antibody does not affect inhibition of MICA/B shedding, as previously reported (Andrade et al., Science 359, 1537-1542 (2018)). Data representative of three independent experiments.

FIG. 32 shows specificity of ELISA assay for MICA compared to MICB. The supernatants of B16F10 cell lines transduced with human MICA (allele 009) or MICB (allele 005) cDNAs were analyzed using an ELISA for MICA (Abcam, Ab59569) that was used throughout this study. These B16F10 cell lines were described previously (Andrade et al., Science 359, 1537-1542 (2018)). The ELISA detected soluble MICA shed by the B16F10-MICA cell line, but not shed MICB released by B16F10-MICB cells.

FIG. 33 shows panobinostat did not inhibit reconstitution of NSG mice with human NK cells and synergized with 7C6 mAb to enhance surface MICA/B expression on melanoma metastases. NSG mice were injected i.v. with 2×10⁶ in vitro expanded human NK cells from healthy donors. Immediately following NK cell inoculation, mice were treated with IL-2 (7.5×10⁴ units) to support NK cell survival; mice also received panobinostat (10 mg/kg in PBS) or PBS as a control. 24 hours later, blood NK cells were analyzed by flow cytometry. (A) Number of circulating NK cells identified as CD45+ CD56+ CD3− viable cells. (B, C) Percentage of blood NK cells labeled with CD16a (B) or NKG2D (C) mAbs. Data pooled from two independent experiments (A-C). (D) Representative histograms of the data shown in FIG. 23A.

FIG. 34 shows genes and the corresponding species and sequences.

### DETAILED DESCRIPTION

Resistance to cytotoxic T cells can arise from mutations in many pathways. For example, resistance to cytotoxic T cells is frequently mediated by loss of MHC class I expression or IFNγ signaling in tumor cells, such as mutations of B2M or JAK1 genes, among others. Activated NK cells can target such resistant tumors, but suitable NK cell-based strategies remain to be developed. Aspects of embodiments described herein address this shortcoming. Specifically, it is shown that B2M and JAK1 deficient metastases were targeted by NK cells following treatment with a mAb that blocked MICA/B shedding, a frequent evasion mechanism in human cancers. Exemplary mAbs can be found in WO2018217688A1, which is incorporated by reference herein in its entirety.

Further, single cell analysis of NK cells in human melanoma metastases, including patients who progressed following checkpoint blockade, identified major transcriptional differences between tumor-infiltrating and circulating NK cells. NK cells are present in most human melanoma metastases, including patients who failed therapy with PD-1 or CTLA-4 mAbs. These cells have transcriptional programs that reflect important functions, such as cytotoxicity, and secretion of chemokines that recruit key immune cell populations required for T cell mediated tumor immunity, such as XCL1 and XCL2 which recruit dendritic cells that express the relevant receptor (XCR1).

Finally, the gene expression programs of seven tumor-infiltrating NK cell clusters indicate significant specialization, including cytotoxicity and chemokine secretion. NK cell-based immunotherapy therefore provides an opportunity to target tumors with mutations that render them resistant to cytotoxic T cells.

### Abbreviations and Definitions

Detailed descriptions of one or more embodiments are provided herein. However, these embodiments can be embodied in various forms. Therefore, specific details disclosed herein are not to be interpreted as limiting, but rather as a basis for the claims and as a representative basis for teaching one skilled in the art to employ embodiments described herein in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Methods of Treatment

Aspects of embodiments described herein are directed towards methods of treating a cell proliferative disorder, such as cancer. More specifically, aspects of embodiments described herein are directed towards methods of treating checkpoint blockade resistant cancer, such as those cancers resistant to cytotoxic T cells.

The terms “cancer” and “cancerous” can refer to or describe the physiological condition in mammals that is typically characterized by unregulated cell growth, as well as any of a number of characteristic structural and/or molecular features. A “cancerous cell” is understood as a cell having specific structural properties, lacking differentiation and in many instances, being capable of invasion and metastasis, see DeVita, V. et al. (eds.), 2001, Cancer Principles and Practice of Oncology, 6th. Ed., Lippincott Williams & Wilkins, Philadelphia, Pa.). The term cancer includes, for example, cancers of the female reproductive organs including, for example, ovarian cancer, cervical cancer and uterine cancer; lung cancer; breast cancer; renal cell carcinoma; Hodgkin's lymphoma; Non-Hodgkin's lymphoma; cancers of the genitourinary system including, for example, kidney cancer, prostate cancer, bladder cancer, and urethral cancer; cancers of the head and neck; liver cancer; cancers of the gastrointestinal system including, for example, stomach cancer, esophageal cancer, small bowel cancer or colon cancer; cancers of the biliary tree; pancreatic cancer; cancers of the male reproductive system including, for example, testicular cancer; Gestational trophoblastic disease; cancers of the endocrine system including, for example, thyroid cancer, parathyroid cancer, adrenal gland cancer, carcinoid tumors, insulinomas and PNET tumors; sarcomas, including, for example, Ewing's sarcoma, osteosarcoma, liposarcoma, leiomyosarcoma, and rhabdomyosarcoma; mesotheliomas; cancers of the skin; melanomas; cancers of the central nervous system; pediatric cancers: and cancers of the hematopoietic system including, for example, all forms of leukemia, myelodysplastic syndromes, myeloproliferative disorders and multiple myeloma. Cancers can also include, for example urological cancers, such as bladder cancer; carcinomas, such as bladder, breast, cervical, cholangiocarcinoma, colorectal, esophageal, gastric, head and neck, kidney, liver, lung. ‘nasopharyngeal, ovarian, pancreas/gall bladder, prostrate and thyroid carcinomas; musculoskeletal carcinomas, such as, osteosarcoma, synovial sarcoma, and rhabdomyosarcoma; soft tissue sarcomas, such as, MFH/fibrosarcoma, leiomyosarcoma, and kaposi's sarcoma; haematopietic malignancies, such as, multiple myeloma, lymphomas, adult T-cell leukemia, acute myelogenous leukemia, and chronic myeloid leukemia; and other neoplasms, such as glioblastomas, astrocytomas, melanoma, mesothelioma, and Wilms' tumor (Birchmeier et al., Nat Rev MoI Cell Bio 2003 4(12):912-925.

The terms “cell proliferative disorder” and “proliferative disorder” can refer to disorders that are associated with some degree of abnormal cell proliferation.

The term “tumor” can refer to all neoplastic cell growth and proliferation, whether malignant or benign, and all pre-cancerous and cancerous cells and tissues.

Unless otherwise indicated by context, the term “cancer”, as used herein, can refer to cancer, cell proliferative disorder or tumor. Similarly, the term “cancer cells” can refer to cells of a cancer, cell proliferative disorder or tumor, unless otherwise indicated by context.

Unless otherwise indicate by context, the terms “cancer”, “cell proliferative disorder” or “tumor” can be used interchangeably.

The terms “treat” or “treatment” can refer to both therapeutic treatment and prophylactic or preventative measures, wherein the desired outcome is to prevent, slow down (lessen), or reverse an undesired physiological change or disorder, such as the progression of cancer. Beneficial or desired clinical results can include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment can include those already with the condition or disorder as well as those prone to have the condition or disorder or those in which the condition or disorder is to be prevented.

In the context of cancer, for example, the term “treating” can include any or all of: preventing growth, proliferation, or metastasis of tumor cells, cancer cells, or of a tumor; preventing replication of tumor cells or cancer cells, lessening of overall tumor burden or decreasing the number of cancerous cells, and ameliorating one or more symptoms associated with the disease.

Embodiments described herein provides for both prophylactic and therapeutic methods of treating a subject suffering from, at risk of, or susceptible to a cancer, or other cell proliferation-related diseases or disorders. For example, the methods are used to treat, prevent or alleviate a symptom of cancer. In an embodiment, the methods are used to treat, prevent or alleviate a symptom of a solid tumor. Non-limiting examples of other tumors that can be treated by embodiments herein comprise lung cancer, ovarian cancer, prostate cancer, colon cancer, bladder cancer, renal cancer, breast cancer, cervical cancer, brain cancer, skin cancer, liver cancer, pancreatic cancer or stomach cancer. Additionally, the methods of Embodiments described herein can be used to treat hematologic cancers such as leukemia and lymphoma, such as Hodgkin's lymphoma. Alternatively, the methods can be used to treat, prevent or alleviate a symptom of a cancer that has metastasized.

In an embodiment, Embodiments described herein provides for methods of treating a subject suffering from, at risk of, or susceptible to a cancer that is resistant to cytotoxic T cells and/or T cell-based therapies (such as checkpoint blockade). For example, cytotoxic T cells play a central role in the efficacy of checkpoint blockade based on their ability to recognize tumor-derived peptides bound to major histocompatibility complex class I (MHC-I) proteins. Recognition of such MHC-I—peptide complexes by the T cell receptor (TCR) triggers the release of interferon-γ (IFNγ) by T cells which inhibits tumor cell proliferation and enhances expression of MHC-I proteins on both tumor and dendritic cells. Resistance to checkpoint blockade is therefore frequently mediated by loss of MHC-I expression by tumor cells, either by mutation or epigenetic silencing of key genes in the MHC-I (B2M, TAP1, TAP2 and other genes) or IFNγ (JAK1, JAK2) pathways. A low number or loss of neoantigens also diminishes tumor immunity mediated by cytotoxic T cells.

Accordingly, in one aspect, Embodiments described herein provides methods for preventing, treating or alleviating a symptom cancer or a cell proliferative disease or disorder in a subject by administering to the subject a monoclonal antibody or fragment or derivative thereof (for example, an scFv antibody or a bi-specific antibody) that activates an anti-tumor NK cell response. An activated NK cell response can be determined by, for example, analysis of tumor biopsy (such as comparing pre-treatment biopsy to post-treatment biopsy). Such analysis can include, for example, multi-color immunofluorescence for granzyme A and perform, together with NK cell marker, such as NKp46 and CD56.

In an embodiment, an anti-MICA/B antibody can be administered to the subject. Many human cancers express the MHC-I polypeptide-related sequence A (MICA) and MICB (MICA/B) proteins that serve as ligands for the activating NK group 2D (NKG2D) receptor on NK cells and subpopulations of T cells. However, tumors frequently evade NKG2D receptor-mediated tumor immunity by proteolytic shedding of MICA/B proteins. The α3 domain of MICA/B is a domain essential for shedding, and monoclonal antibodies that bind to this domain can inhibit MICA/B shedding and induced NK cell-mediated tumor immunity. The increased density of MICA/B proteins on tumor cells enhanced NKG2D receptor-mediated activation in NK cells, and the Fc segment of tumor-bound antibodies also activated NK cells through the CD16 Fc receptor. Treatment with such MICA/B antibodies induced a striking shift of tumor-infiltrating NK cells to a highly cytotoxic state.

Non-limiting examples of an anti-MICA/B antibodies that can be utilized in embodiments herein include any antibody that is specific for anti-MICA/B. In an embodiment, the antibody is specific for the 3 domain of MICA/B. See, for example, WO2018217688, which is incorporated by reference herein in its entirety.

Therefore, embodiments described herein comprise antibodies, such as monoclonal antibodies, such as human monoclonal antibodies, that specifically bind MHC class I polypeptide-related sequence A (MICA) and/or B (MICB) a3 domain, the site of proteolytic shedding and have desirable functional properties. These properties include inhibition of MICA/B shedding by human cancer cells, stabilization of cell surface MICA/B for NK cell recognition, and activation of both NKG2D and CD16 Fc receptors on NK cells. MICA antibodies with these properties restore immune activation by stress molecules that activate cytotoxic lymphocytes.

In some embodiments, the monoclonal antibodies, or antigen binding portions thereof, which bind to MICA and/or MICB comprise heavy and light chain variable regions, wherein the heavy chain CDR1, CDR2, and CDR3 sequences comprise SEQ ID NOs: 1-3, respectively, as displayed in Table 1. In some embodiments, the monoclonal antibodies, or antigen binding portions thereof, comprise heavy and light chain variable regions wherein light chain CDR1, CDR2, and CDR3 sequences comprise SEQ ID NOs: 4-6, respectively, as displayed in Table 1. In some embodiments, the monoclonal antibodies, or antigen binding portions thereof, which bind to MICA and/or MICB comprise heavy and light chain variable regions, the heavy chain CDR1, CDR2, and CDR3 sequences comprising SEQ ID NOs: 1-3, respectively, and the light chain CDR1, CDR2 and CDR3 sequences comprising SEQ ID NOs: 4-6.

Provided herein are isolated monoclonal antibodies, or antigen binding portions thereof, which bind to MICA and/or MICB and comprise a heavy and light chain variable regions, wherein the heavy chain variable region comprises an amino acid sequence which is at least 90%, 95A, 96%, 97%, 98%, 99%, or 1000 identical to the amino acid sequence set forth in SEQ ID NO: 7.

Provided herein are isolated monoclonal antibodies, or antigen binding portions thereof, which bind to MICA and/or MICB and comprise heavy and light chain variable regions, wherein the light chain variable region comprises an amino acid sequence which is at least 90%, 95%, 96%, 97%, 98%, 99%, or 100% identical to the amino acid sequence set forth in SEQ ID NO: 8.

TABLE 1

| | | | |
|---|---|---|---|
| Heavy Chain | Amino acid | NYAMN | SEQ ID NO: 1 |
| CDR1 | sequence | | |
| | Nucleotide | AACTATGCAATGAAC | SEQ ID NO: 9 |
| | sequence | | |
| Heavy Chain | Amino acid | WINTHTGDPTYADDFKG | SEQ ID NO: 2 |
| CDR2 | sequence | | |
| | Nucleotide | TGGATAAACACCCACACTG | SEQ ID NO: 10 |
| | sequence | GAGACCCAACATATGCTGA | |
| | | TGACTTCAAGGGA | |
| Heavy Chain | Amino acid | TYGNYAMDY | SEQ ID NO: 3 |
| CDR3 | sequence | | |
| | Nucleotide | ACTTATGGTAATTACGCTA | SEQ ID NO: 11 |
| | sequence | TGGACTAC | |
| Light Chain | Amino acid | SASQDISNYLN | SEQ ID NO: 4 |
| CDR1 | sequence | | |
| | Nucleotide | AGTGCAAGTCAGGACATTA | SEQ ID NO: 12 |
| | sequence | GCAATTATTTAAAC | |
| Light Chain | Amino acid | DTSILHL | SEQ ID NO: 5 |
| CDR2 | sequence | | |
| | Nucleotide | GACACATCAATTTTACAC | SEQ ID NO: 13 |
| | sequence | TTA | |
| Light Chain | Amino acid | QQYSKFPRT | SEQ ID NO: 6 |
| CDR3 | sequence | | |
| | Nucleotide | CAGCAGTATAGTAAAT | SEQ ID NO: 14 |
| | sequence | TTCCTCGGACG | |
| Heavy Chain | Amino acid | QIQLVQSGPELKKPGETVKV | SEQ ID NO: 7 |
| variable | sequence | SCKASGYMFTNYAMNWVK | |
| region | | QAPEKGLKWMGWINTHTGD | |
| | | PTYADDFKGRIAFSLETSAS | |
| | | TAYLQINNLKNEDTATYFCV | |
| | | RTYGNYAMDYWGQGTSVT | |
| | | VSSAKTTAPSVYPLAPVCGD | |
| | | TTGSSVTLGCLVKGYFPEPV | |
| | | TLTWNSGSLSSGVHTFPAVL | |
| | | QSDLYTLSSSVTVTSS | |
| | Nucleotide | CAGATCCAGTTGGTGCAGT | SEQ ID NO: 15 |
| | Sequence | CTGGACCTGAGCTGAAGAA | |
| | | GCCTGGAGAGACAGTCAAG | |
| | | GTCTCCTGCAAGGCTTCTG | |
| | | GGTATATGTTCACAAACTA | |
| | | TGCAATGAACTGGGTGAAG | |
| | | CAGGCTCCAGAAAAGGGTT | |
| | | TAAAGTGGATGGGCTGGAT | |
| | | AAACACCCACACTGGAGAC | |
| | | CCAACATATGCTGATGACT | |
| | | TCAAGGGACGAATTGCCTT | |
| | | CTCTTTGGAAACCTCTGCC | |
| | | AGCACTGCCTATTTGCAGA | |
| | | TCAACAACCTCAAAAATGA | |
| | | GGACACGGCTACATATTTC | |
| | | TGTGTAAGAACTTATGGTA | |
| | | ATTACGCTATGGACTACTG | |
| | | GGGTCAAGGAACCTCAGTC | |
| | | ACCGTCTCCTCAGCCAAAA | |
| | | CAACAGCCCCATCGGTCTA | |
| | | TCCACTGGCCCCTGTGTGT | |
| | | GGAGATACAACTGGCTCCT | |
| | | CGGTGACTCTAGGATGCCT | |
| | | GGTCAAGGGTTATTTCCCT | |
| | | GAGCCAGTGACCTTGACCT | |
| | | GGAACTCTGGATCCCTGTC | |
| | | CAGTGGTGTGCACACCTTC | |
| | | CCAGCTGTCCTGCAGTCTG | |
| | | ACCTCTACACCCTCAGCAG | |
| | | CTCAGTGACTGTAACCTCG | |
| | | AGC | |
| Light Chain | Amino acid | DIQMTQTTSSLSASLGDRVTI | SEQ ID NO: 8 |
| variable | sequence | SCSASQDISNYLNWYQQKPD | |
| region | | GTVKLLIYDTSILHLGVPSR | |
| | | FSGSGSGTDYSLTISNLEPEDI | |
| | | ATYYCQQYSKFPRTFGGGTT | |
| | | LEIK | |
| | Nucleotide | GATATCCAGATGACACAGA | SEQ ID NO: 16 |
| | Sequence | CCACATCCTCCCTGTCTGCC | |
| | | TCTCTGGGAGACAGAGTCA | |
| | | CCATCAGTTGCAGTGCAAG | |
| | | TCAGGACATTAGCAATTAT | |
| | | TTAAACTGGTATCAGCAGA | |
| | | AACCAGATGGAACTGTTAA | |
| | | ACTCCTGATCTATGACACA | |
| | | TCAATTTTACACTTAGGAG | |
| | | TCCCATCAAGGTTCAGTGG | |
| | | CAGTGGGTCTGGGACAGAT | |
| | | TATTCTCTCACCATCAGTAA | |
| | | CCTGGAACCTGAAGATATT | |
| | | GCCACTTACTATTGTCAGC | |
| | | AGTATAGTAAATTTCCTCG | |
| | | GACGTTCGGTGGAGGCACC | |
| | | ACGCTGGAAATCAAA | |

For example, the antibody is includes clone 7C6 or example 7C6-hIgG1), 6F11, and/or 1C2.

In a related aspect, embodiments herein can comprise nucleic acids encoding the heavy and/or light chain variable regions of the anti-MICA and/or anti-MICB antibodies, or antigen binding portions thereof, expression vectors comprising the nucleic acid molecules, and cells transformed with the expression vectors. Also, embodiments herein can further comprise methods of preparing the anti-MICA and/or anti-MICB antibodies, comprising expressing an anti-MICA and/or anti-MICB antibody in a cell and isolating the antibody from the cell.

Also provided herein are compositions comprising anti-MICA and/or anti-MICB antibodies, or antigen binding portions thereof, and a carrier. Also provided herein are immunoconjugates comprising the anti-MICA and/or anti-MICB antibodies described herein, linked to an agent. Also provided herein are kits comprising the anti-MICA and/or anti-MICB antibodies, or antigen binding portions thereof, and instructions for use.

In embodiments, the antibodies can be administered in a therapeutically effective amount as described further herein.

The terms “patient” or “subject” can be used interchangeably. Examples of a “patient” or “subject” include, but are not limited to, a human, rat, mouse, guinea pig, monkey, pig, goat, cow, horse, dog, cat, bird and fowl. In an exemplary embodiment, the patient is a human.

A “cancer patient” can refer to an individual that has been diagnosed as having cancer. Examples of cancers include, but are not limited to, a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye, thyroid, adrenal gland, and other endocrine glands and related structures, secondary and unspecified malignant neoplasms of lymph nodes, secondary malignant neoplasm of respiratory and digestive systems and secondary malignant neoplasm of other sites. In an embodiment, said cancer is melanoma, lung cancer, such as non-small-cell lung cancer, prostate cancer, renal-cell cancer or colorectal cancer.

Subjects at risk for or susceptible to cancer or cell proliferation-related diseases or disorders can include patients who have a family history of cancer or a subject exposed to a known or suspected cancer-causing agent. Administration of a prophylactic agent can occur prior to the manifestation of cancer such that the disease is prevented or, alternatively, delayed in its progression. Administration of a therapeutic agent can occur once a subject has been diagnosed with cancer such that the progression of the disease is reversed, delayed, or stopped.

In one aspect, embodiments described herein provides methods for preventing, treating or alleviating a symptom of cancer or a cell proliferative disease or disorder in a subject by administering to the subject a monoclonal antibody or fragment or derivative thereof (for example, an scFv antibody or a bi-specific antibody) that activates an anti-tumor NK cell response. As described herein, the term “treating” can include any or all of: preventing growth, proliferation, or metastasis of tumor cells, cancer cells, or of a tumor; preventing replication of tumor cells or cancer cells, lessening of overall tumor burden or decreasing the number of cancerous cells (such as by inducing cell lysis), and ameliorating one or more symptoms associated with the disease.

As used herein, “metastasis” can refer to the distant spread of a malignant tumor from its sight of origin. Cancer cells can metastasize through the bloodstream, through the lymphatic system, across body cavities, or any combination thereof.

The term “cell proliferation” can refer to a relative increase in cell number, whether by cell division or by inhibition of cell death (e.g., necrosis, apoptosis), for example. Inappropriate cell proliferation can result from, for example, inappropriate cell growth, an excessive cell division, cell division (i.e., mitosis), and/or inappropriate cell survival.

The term “cell lysis” can refer to the disintegration of cells by destruction of walls or membranes. For example, the breaking down of the cell (i.e., lysis) can be by viral, chemical, enzymic, or osmotic mechanisms that compromise the integrity of the cell.

In another aspect, embodiments described herein provides methods for sensitizing a cancer or cancer cells to the anti-cancer effect of Natural killer cells. Natural killer cells (also known as NK cells, K cells, and/or killer cells) are a type of lymphocyte that plays a role in the host-rejection of tumors and virtually infected cells. Natural Killer (NK) cells recognize tumor cells by molecular mechanisms that differ substantially from those required by cytotoxic T cells. NK cell recognition of tumor cells is mediated by ligands associated with malignant transformation, including DNA damage and cellular stress. Without wishing to be bound by theory, tumors resistant to cytotoxic T cells can respond to NK cell-based immunotherapy approaches.

Activation of natural killer (NK) cells is dictated by a balance between negative signals provided by inhibitory receptors upon interaction with major histocompatibility complex (MHC) class I molecules and positive signals promoted by a variety of activating receptors. NK cells express a wide array of activating receptors that cooperate in driving the natural cytotoxic response. These receptors include the natural cytotoxicity receptors (NCRs), the signaling lymphocyte activation molecule (SLAM) family receptor member, 2B4, the Ig-like receptor DNAX accessory molecule-1 (DNAM1), and the lectin-like receptor natural-killer receptor group 2, member D (NKG2D), NKp46 and CD16a (receptor for IgG).

NKG2D is a potent activating receptor constitutively expressed on all NK cells but is also present on invariant natural killer T (NKT) cells and subsets of T cells including CD8⁺ αβ T cells, and γδ T cells. It can bind several ligands poorly expressed on healthy cells but is upregulated upon stressing stimuli in the context of cancer or viral infection. Several in vivo models indicate a fundamental role for the NKG2D receptor in NK cell responses toward abnormal cells.

The most remarkable characteristic of NKG2D receptor resides in its ability to bind to a large repertoire of self-proteins induced by stress pathways, thus mediating the “induced self” recognition. In humans, these ligands include the highly polymorphic MHC class I related proteins (MIC)A and MICB, and 6 members of UL16 binding proteins (ULBP). NKG2D ligands (NKG2DLs) are absent on the surface of the vast majority of healthy tissues but are upregulated under stressing conditions, including mitosis, viral infection and cancer by several pathways mainly acting at transcriptional and post-transcriptional levels.

Many human cancers express the MHC-I polypeptide-related sequence A (MICA) and MICB (MICA/B) proteins that serve as ligands for the activating NK group 2D (NKG2D) receptor on NK cells and subpopulations of T cells. However, tumors frequently evade NKG2D receptor-mediated tumor immunity by proteolytic shedding of MICA/B proteins. The 3 domain of MICA/B is a domain essential for shedding, and monoclonal antibodies that bind to this domain can inhibit MICA/B shedding and induced NK cell-mediated tumor immunity. The increased density of MICA/B proteins on tumor cells enhanced NKG2D receptor-mediated activation in NK cells. Treatment with such MICA/B antibodies induced a striking shift of tumor-infiltrating NK cells to a highly cytotoxic state.

In addition to NKG2D, the Fc segment of tumor-bound antibodies also activated NK cells through the CD16 Fc receptor. Upon IgG binding, CD16 initiates signaling cascades that produce a diverse variety of responses including antibody-dependent cell-mediated cytotoxicity (ADCC), degranulation, and cytokine secretion. CD16 is expressed on macrophages, natural killer (NK) cells and neutrophils. In this context, its expression on NK cells is of particular relevance.

Referring to the examples, MHC class I deficient tumor cells (i.e., tumor resistant to cytotoxic T cells) are efficiently killed in the presence of activating agents such as MICA antibodies which activate NK cells through NKG2D and CD16. Thus, tumors resistant to cytotoxic T cells can be targeted through NK cell activation through NKG2D and CD16.

Aspects of embodiments described herein comprise activating agents and the use thereof to activate the anti-cancer NK cell response. The term “activating agent” can refer to any agent that enables the activation of NK cells. For example, the activating agent can activate NK cells through NKG2D and/or CD16. For example, the activating agent can be a polynucleotide, a polypeptide, a biologic, a cytokine (for example, IL-15), or a small molecule. Molecular markers of activation of NK cells are known in the art, and include high level expression of cytotoxicity proteins (perform, granzyme A) and cytokines (such as IFN gamma). In an embodiment, the activating agent is an antibody or fragment thereof, such as an anti-MICA/B antibody. In an embodiment, the activating agent is an antibody or fragment thereof as described in Table 1.

Aspects of embodiments described herein are useful for preventing, treating, or alleviating a symptom of a drug-resistant cancer. As used herein, a “drug resistant” or “refractory” cancer, cell proliferative disorder or tumor can refer to a refractory cancer, cell proliferative disorder or tumor which cells exhibit reduced cytotoxicity to a drug as compared to a comparable, sensitive cells. For example, the tumor and/or cancer cell can be resistant to cytotoxic T cells, such as that induced by checkpoint blockade therapy.

For example, aspects of embodiments described herein are useful for preventing, treating, or alleviating a symptom of a cancer resistant to cytotoxic T cells and checkpoint blockade, such as a cancer resistant to immunotherapies that activate T cells. For example, the cancer can be resistant to anti-CTLA4, anti-PD1, and/or anti-PDL1 antibodies. Immune checkpoints are inhibitory pathways that slow down or stop immune reactions and prevent excessive tissue damage from uncontrolled activity of immune cells. “Checkpoint blockade (CPB) therapy” can refer to therapy that inhibits the inhibitory pathways, allowing more extensive immune activity. Such therapy can comprise treatment with any small molecule chemical compound, antibody, nucleic acid molecule, or polypeptide, or fragment thereof, that inhibits the inhibitory pathways. “Checkpoint blockade therapy” can also refer to stimulation of a preexisting immune response. In certain embodiments, CPB therapy is therapy with an inhibitor of the programmed death-1 (PD-1) pathway, for example an anti-PD1 antibody, such as, but not limited to Nivolumab. In other embodiments, CPB therapy is therapy with an anti-cytotoxic T-lymphocyte-associated antigen (CTLA-4) antibody. In additional embodiments, the CPB therapy is targeted at another member of the CD28CTLA4 Ig superfamily such as BTLA, LAG3, ICOS, PDL1 or KIR (Page et al., Annual Review of Medicine 65:27 (2014)). In further additional embodiments, the CPB therapy is targeted at a member of the TNFR superfamily such as CD40, OX40, CD 137, GITR, CD27 or TEVI-3. In some cases, targeting a checkpoint inhibitor is accomplished with an inhibitory antibody or similar molecule. In other cases, it is accomplished with an agonist for the target; examples of this class include the stimulatory targets OX40 and GITR. For example, CPB therapy comprising therapy with antibodies selected from anti-CTLA4, anti-PD1, anti-PDL1 antibodies and a combination thereof.

The nature of the CPB therapy is not critical to embodiments described herein and examples of suitable agents are described herein. In embodiments, the CPB therapy is therapy with antibodies selected from anti-CTLA4, anti-PD1, anti-PDL1 antibodies and a combination thereof. An exemplary anti-CTLA4 antibody is ipilimumab. An exemplary anti-PD1 antibody is nivolumab. A significant number of cancer patients undergoing CPB therapy, after a first period of regression, become resistant to CPB therapy resulting in progression of the tumor. Resistance to CPB therapy is linked to reduced expression of a gene relating to antigen processing pathway or a product thereof, such as B2M and Jak1, among others. As described herein, B2M and JAK1 deficient metastases were targeted by NK cells following treatment with a mAb that blocked MICA/B shedding, a frequent evasion mechanism in human cancers.

Aspects of embodiments described herein are also useful for preventing, treating, or alleviating a symptom of a cancer resistant to cytotoxic T cells. “T cell” refers to T lymphocytes, and includes, but is not limited to, γ:δ⁺ T cells, NK T cells, CD4+ T cells and CD8+ T cells. CD4+ T cells include T_{H}0, T_{H}1 and T_{H}2 cells, as well as regulatory T cells (T_{reg}). There are at least three types of regulatory T cells: CD4+ CD25+ T_{reg}, CD25⁻ T_{H}0 T_{reg}, and CD25⁻ T_{R}1 T_{reg}. “Cytotoxic T cell” refers to a T cell that can kill another cell. The majority of cytotoxic T cells are CD8+ MHC class I-restricted T cells, however some cytotoxic T cells are CD4+.

Most T cell receptors (TCRs) recognize the complex of a peptide antigen (or a peptide fragment of an antigen) bound to an MHC molecule (MHC:antigen complex). The TCR is responsible for the antigen specificity of each T cell, as well as for restriction to recognition of antigen displayed by MHC class I molecules versus MHC class II molecules. TCRs originating in CD4+ T cells are MHC class II restricted, meaning that TCRs originating from CD4+ T cells only recognize antigen displayed by MHC class II molecules. TCRs originating from CD8+ T cells are MHC class I restricted, and only recognize antigen displayed by MHC class I molecules.

Without wishing to be bound by theory, tumors resistant to cytotoxic T cells can respond to NK cell-based immunotherapy approaches. In fact, loss of MHC-I expression by tumor cells (also referred to as MHC Class I deficient cancers) render them more sensitive to NK cells because MHC-I proteins serve as ligands for inhibitory NK cell receptors. Thus, aspects of embodiments described herein can also be considered to be useful to treat, prevent, or alleviate a symptom of an MHC Class I deficient cancer.

Aspects of embodiments described herein can also be considered to be useful to treat, prevent, or alleviate a symptom of a cancer resistant to IFN gamma released by T cells. For example, aspects of embodiments described herein can comprise activating NK cells against tumors with one or more loss mutations in gamma interferon pathway.

Aspects of embodiments described herein can also be considered to be useful to treat, prevent, or alleviate a symptom of a cancer that is MHC Class I deficient and also resistant to IFN gamma.

Further, aspects of embodiments described herein are useful for preventing, treating, or alleviating the symptoms of cancer resistant to cancer immunotherapy. Cancer immunotherapy can refer to a diverse set of therapeutic strategies designed to induce the patient's own immune system to fight the tumor.

Several types of immunotherapy are used to treat cancer. These treatments can either help the immune system attack the cancer directly or stimulate the immune system in a more general way.

Types of immunotherapy that help the immune system act directly against the cancer include checkpoint inhibitors, which are drugs that help the immune system respond more strongly to a tumor. These drugs work by releasing the inhibitor pathways that keep T cells from killing cancer cells. These drugs do not target the tumor directly. Instead, they interfere with the ability of cancer cells to avoid immune system attack. Such immunotherapy can include, for example, checkpoint blockade inhibitors, such as anti-CTLA4, anti-PD1 and/or anti-PDL1. Adoptive cell transfer is a treatment that attempts to boost the natural ability of a subject's T cells to fight cancer. In this treatment, T cells are taken from the subject's tumor and then those that are most active against the cancer are grown in large batches in the lab before being administered back to the subject. Adoptive cell transfer can include therapies called CAR T-cell therapy, which uses T cells that are engineered in a laboratory to target a specific cancer. Monoclonal antibodies, also known as therapeutic antibodies, are immune system proteins produced in the lab. These antibodies are designed to attach to specific targets found on cancer cells. Some monoclonal antibodies mark cancer cells so that they will be better seen and destroyed by the immune system, and these are a type of immunotherapy. Other monoclonal antibodies that are used in cancer treatment do not cause a response from the immune system. Such monoclonal antibodies are considered to be targeted therapy, rather than immunotherapy. Treatment vaccines work against cancer by boosting one's immune system's response to cancer cells.

Other types of immunotherapy that enhance or stimulate the body's immune response to fight the cancer include cytokines, which are proteins made by your body's cells. They play important roles in the body's normal immune responses and also in the immune system's ability to respond to cancer. The two main types of cytokines used to treat cancer are called interferons and interleukins.

Another aspect of embodiments described herein is directed towards compositions and methods for sensitizing a cell to an anti-cancer agent. “Sensitizing” can refer to the ability of an activating agent to increase the sensitivity of a designated system, such as a cell or tumor. This meaning can include changing (i.e., sensitizing) a cell to make it more responsive to an anti-cancer compound or regimen to which it previously was not sensitive or was less sensitive. Sensitizing and “more sensitive” can also include increasing the sensitivity of a cell or tumor such that exposure to a previously non-killing substance results in cell death

In embodiments, one or more activating agent(s) are administered to a subject to treat, prevent, or alleviate a symptom of cancer. The activating agent(s) can be provided in a pharmaceutically acceptable composition that can be in any form that allows for the composition to be administered to a patient. For example, the composition can be in the form of a liquid or solid. Typical routes of administration include, without limitation, oral, topical, parenteral, sublingual, rectal, vaginal, ocular, and intra-tumor. Parenteral administration includes subcutaneous injections, intravenous, intramuscular, intrasternal injection or infusion techniques. In one embodiment, the composition can be administered by infusion using a minipump infusion system.

In an embodiment, the pharmaceutical composition can be administered to a subject as antibody preparation. An antibody preparation, for example, one having high specificity and high affinity for its target antigen, can have an effect due to its binding with the target. For example, the α3 domain of MICA/B is a domain essential for shedding, and monoclonal antibodies that bind to this domain can inhibit MICA/B shedding and induced NK cell-mediated tumor immunity. The increased density of MICA/B proteins on tumor cells enhanced NKG2D receptor-mediated activation in NK cells, and the Fc segment of tumor-bound antibodies also activated NK cells through the CD16 Fc receptor. Treatment with such MICA/B antibodies induced a striking shift of tumor-infiltrating NK cells to a highly cytotoxic state.

Activating agents of embodiments described herein, for example antibodies that specifically bind the α3 domain of MICA/B, can be administered for the treatment of a cancer in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R. Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, the particular disease being treated, and the severity of the particular disease being treated. Judgment of such factors by medical caregivers is within the ordinary skill in the art. The amount will also depend on the individual patient to be treated, the route of administration, the type of formulation, the characteristics of the compound used, the disease being treated, the severity of the disease, and the desired effect. The amount used can be determined by pharmacological and pharmacokinetic principles well known in the art.

The term “effective amount” or “therapeutically effective amount” can refer to an amount of a drug or therapeutic agent (i.e., activating agent) effective to treat (e g, kill) a cancer cell in a mammal. In the case of cancer, the effective amount of the drug can reduce the number of cancer cells; reduce the tumor size; inhibit (i.e., slow to some extent and/or stop) cancer cell infiltration into peripheral organs; inhibit (i.e., slow to some extent and/or stop) tumor metastasis; inhibit, to some extent, tumor growth; and/or relieve to some extent one or more of the symptoms associated with the cancer. To the extent the drug can prevent growth and/or kill existing cancer cells, it can be cytostatic and/or cytotoxic. For cancer therapy, efficacy can, for example, be measured by assessing the time to disease progression (TTP) and/or determining the response rate (RR).

A therapeutically effective amount of the embodiments described herein can be the amount needed to achieve a therapeutic objective. As noted herein, this can be a binding interaction between an antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. As another example, this can be the activation of NK cells, such as the anti-cancer activity of NK cells.

In an embodiment, the dosage of an activating agent administered to a subject (e.g., a patient) is typically 0.1 mg/kg to 100 mg/kg of the patient's body weight, between 0.1 mg/kg and 20 mg/kg of the patient's body weight, or 1 mg/kg to 10 mg/kg of the patient's body weight.

Referring to antibodies, human antibodies have a longer half-life within the human body than antibodies from other species due to the immune response to the foreign polypeptides. Thus, lower dosages of human antibodies and less frequent administration can be used. Further, the dosage and frequency of administration of antibodies can be reduced by enhancing uptake and tissue penetration (e.g., into the brain) of the antibodies by modifications such as, for example, lipidation. Common ranges for therapeutically effective dosing of an antibody or antibody fragment of Embodiments described herein can be, by way of nonlimiting example, from about 0.1 mg/kg body weight to about 50 mg/kg body weight. Common dosing frequencies can range, for example, from twice daily to once a week.

In embodiments, the smallest inhibitory antibody fragment that specifically binds to the binding domain of the target protein can be used. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90: 7889-7893 (1993)). The formulation can also contain more than one active compound as necessary for the indication being treated, for example those with complementary activities that do not adversely affect each other. Alternatively, or in addition, the composition can comprise an agent that enhances its function, such as, for example, a cytotoxic agent, cytokine (e.g. IL-15, IL12, IL18), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. For example, this is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

As described in detail herein, compositions of embodiments described herein as described herein, such as those containing one or more activating agents, can be administered in combination with one or more additional therapeutic agents or therapeutic or prophylactic regimens. For example, the one or more additional therapeutic agent can be a chemotherapeutic agent, a cytokine (such as IL15, IL12, IL18), radiotherapy, or an immunotherapeutic agent.

In additional embodiments, the compositions described herein can be administered in combination with other therapeutic or prophylactic regimens, such as, for example, radiation therapy.

Embodiments described herein provides for methods of treating cancer in a patient by administering two or more antibodies that bind to the same epitope of an antigen or, alternatively, two or more different epitopes of the antigen. Alternatively, the cancer can be treated by administering a first antibody that binds to a first antigen and a second antibody that binds to a protein other than the first antigen. For example, the first antibody can bind to MICA/B, and the second antibody can bine to PD1, PDL1, CTLA4, or a combination thereof.

In other embodiments, the cancer can be treated by administering a bispecific antibody that binds to a first antigen and that binds to a protein other than the first antigen.

In some embodiments, embodiments described herein provides for the administration of a first antibody or activating agent alone or in combination with a second activating agent or antibody that recognizes another protein other than that recognized by the first antibody, with cells that can effect or augment an immune response. For example, these cells can be peripheral blood mononuclear cells (PBMC), or any cell type that is found in PBMC, e.g., cytotoxic T cells, macrophages, and natural killer (NK) cells.

Additionally, embodiments described herein provides administration of one or more activating agents and an anti-neoplastic agent, such a small molecule, a growth factor, a cytokine, or other therapeutics including biomolecules such as peptides, peptidomimetics, peptoids, polynucleotides, lipid-derived mediators, small biogenic amines, hormones, neuropeptides, and proteases. Small molecules include, but are not limited to, inorganic molecules and small organic molecules. Suitable growth factors or cytokines include an IL-2, IL12, IL15, IL18, GM-CSF, IL-12, and TNF-alpha. Small molecule libraries are known in the art. (See, Lam, Anticancer Drug Des., 12: 145, 1997.)

An embodiment also comprises (a) evaluating the patient to determine if the patient has a refractory or drug resistant cancer, or cancer resistant to cytotoxic T cells; (b) administering an effective amount of one or more activating agents to the patient; and (c) monitoring the patient to determine the status of the cancer.

For example, a biological sample from the subject can be evaluated for a marker of resistance to cytotoxic T cells, such as a Jak1 or B2M mutation or any other mutations that abrogate the function of the IFN gamma signaling pathway (such as Stat1 mutation) or the MHC class I antigen presentation pathway in tumor cells (such as Tap1 or Tap2 mutation). The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of Embodiments described herein can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations.

The step of evaluating and/or monitoring the patient or the patient's cancer can comprise the use of a probe for detecting the presence of a cellular marker in a sample. For example, the probe can contain a detectable label. In embodiments, the probe is an antibody, but can also be a polynucleotide or small molecule. Antibodies can be polyclonal or monoclonal. An intact antibody, or a fragment thereof (e.g., Fab, scFv, or F(ab)2) can be used. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Detection can be facilitated by coupling (i.e., physically linking) the probe or antibody to a detectable substance. Examples of detectable substances include, but are not limited to, various enzymes, prosthetic groups, fluorescent materials, luminescent materials, bioluminescent materials, and radioactive materials. Non-limiting examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P or ³H.

Compositions for Treating Cancer

Aspects of embodiments described herein are also directed towards compositions for preventing, treating or alleviating a symptom of cancer or a cellular proliferative disease.

Further, aspects of embodiments described herein are directed towards compositions for preventing growth, proliferation, or metastasis of tumor cells, cancer cells, or of a tumor; preventing replication of tumor cells or cancer cells, lessening of overall tumor burden or decreasing the number of cancerous cells, and ameliorating one or more symptoms associated with the disease.

In embodiments, the compositions comprise one or more activating agents as described herein. For example, the composition can comprise an antibody or fragment thereof as described in Table 1. For example, the composition can comprise cytokines, such as IL-2, IL-3, IL-4, IL-5, IL-6, IL-7, IL-10, IL-12, IL-13, IL-15, IL-18, anti-CD40, CD40L, and TNF-α. In embodiments, the cytokine is IL-15.

The compositions are suitable for veterinary or human administration. The compositions of embodiments described herein can be in any form that allows for the composition to be administered to a human or an animal. For example, the composition can be in the form of a solid, liquid or gas (aerosol). Typical routes of administration include, without limitation, oral, topical, parenteral, sublingual, rectal, vaginal, ocular, and intranasal. Parenteral administration includes subcutaneous injections, intravenous, intramuscular, intrasternal injection or infusion techniques. For example, the compositions are administered parenterally. Pharmaceutical compositions of embodiments described herein can be formulated so as to allow the activating agent, such as an anti-MICA/B antibody, to be bioavailable upon administration of the composition to an animal. Compositions can take the form of one or more dosage units, where for example, a tablet can be a single dosage unit, and a container of an activating agent in aerosol form can hold a plurality of dosage units.

Materials used in preparing the pharmaceutical compositions can be nontoxic in the amounts used. It will be evident to those of ordinary skill in the art that the optimal dosage of the active ingredient(s) in the pharmaceutical composition, such as the activating agent, will depend on a variety of factors. Relevant factors include, without limitation, the type of animal (e.g., human), the particular form of the activating agent, the particular disease to be treated or prevented, the manner of administration, and the composition employed.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

When intended for oral administration, the composition can be in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it can contain, in addition to materials of the above type, a liquid carrier such as polyethylene glycol, cyclodextrin or a fatty oil.

The composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The liquid compositions of embodiments described herein, whether they are solutions, suspensions or other like form, can also include one or more of the following: sterile diluents such as water for injection, saline solution, such as physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline can be an adjuvant. An injectable composition can be sterile.

The amount of the composition that is effective in the treatment of a disorder or condition will depend on the nature of the disorder or condition, and can be determined by standard clinical techniques. In addition, in vitro or in vivo assays can be employed to help identify optimal dosage ranges. The precise dose to be employed in the compositions will also depend on the route of administration, and the seriousness of the disease or disorder, and can be decided according to the judgment of the practitioner and each patient's circumstances.

The compositions can comprise an effective amount of at least one activating agent such that a suitable dosage will be obtained. For example, this amount is at least about 0.01% of by weight of the composition. When intended for oral administration, this amount can be varied to range from about 0.10% to about 80% by weight of the composition. Oral compositions can comprise from about 4% to about 50% by weight of the composition. Compositions of embodiments described herein can be prepared so that a parenteral dosage unit contains from about 0.01% to about 2% by weight of the activating agent.

For intravenous administration, the composition can comprise from about 1 to about 250 mg of an activating agent per kg of the animal's body weight. For example, the amount administered will be in the range from about 4 to about 25 mg/kg of body weight of the activating agent.

In embodiments, the dosage of the activating agent administered to an animal is typically about 0.1 mg/kg to about 1000 mg/kg of the animal's body weight. For example, the dosage of the activating agent administered to an animal is typically about 0.1 mg/kg to about 250 mg/kg of the animal's body weight. For example, the dosage administered to an animal is between about 0.1 mg/kg and about 20 mg/kg of the animal's body weight, such as about 1 mg/kg to about 10 mg/kg of the animal's body weight.

The compositions can be administered by any convenient route, for example by infusion or bolus injection, by absorption through epithelial or mucocutaneous linings (e.g., oral mucosa, rectal and intestinal mucosa, etc.). Administration can be systemic or local. Various delivery systems are known, e.g., encapsulation in liposomes, microparticles, microcapsules, capsules, etc., and can be used to administer a composition. In certain embodiments, more than one activating agent or composition is administered to an animal. Methods of administration include, but are not limited to, oral administration and parenteral administration; parenteral administration including, but not limited to, intradermal, intramuscular, intraperitoneal, intravenous, subcutaneous; intranasal, epidural, sublingual, intranasal, intracerebral, intraventricular, intrathecal, intravaginal, transdermal, rectally, by inhalation, or topically to the ears, nose, eyes, or skin. In embodiments, the mode of administration can be left to the discretion of the practitioner, and will depend in-part upon the site of the medical condition (for example, the site of the cancer or intratumorally).

In an embodiment, the activating agent(s) or compositions are administered parenterally.

In an embodiment, the activating agent(s) or compositions are administered intravenously, such as by an infusion pump or drip.

In specific embodiments, one or more activating agents or compositions can be administered locally to the area in need of treatment. This can be achieved, for example, and not by way of limitation, by local infusion during surgery; topical application, e.g., in conjunction with a wound dressing after surgery; by injection; by means of a catheter; by means of a port; by means of a suppository; or by means of an implant, the implant being of a porous, non-porous, or gelatinous material, including membranes, such as sialastic membranes, or fibers. In one embodiment, administration can be by direct injection at the site (or former site) of a cancer, tumor or neoplastic or pre-neoplastic tissue. In another embodiment, administration can be by direct injection at the site (or former site) of manifestation of disease.

Pulmonary administration can also be employed, e.g., by use of an inhaler or nebulizer, and formulation with an aerosolizing agent, or via perfusion in a fluorocarbon or synthetic pulmonary surfactant. In certain embodiments, the activating agent or compositions can be formulated as a suppository, with traditional binders and carriers such as triglycerides.

In another embodiment, the activating agent can be delivered in a vesicle, such as a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see ibid.)

In yet another embodiment, the activating agent or compositions can be delivered in a controlled release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled-release system can be placed in proximity of the target of the activating agent or compositions, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 249:1527-1533 (1990)) can be used.

The term “carrier” can refer to a diluent, adjuvant or excipient, with which an activating agent is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the activating agent and pharmaceutically acceptable carriers are sterile. Water can be a carrier when the activating agent is administered intravenously. Saline solutions and aqueous dextrose and glycerol solutions can also be employed as liquid carriers, such as for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions can also contain minor amounts of wetting or emulsifying agent, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in “Remington's Pharmaceutical Sciences” by E. W. Martin.

In an embodiment, the activating agent(s) are formulated in accordance with routine procedures as a pharmaceutical composition adapted for intravenous administration to animals, such as human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. In embodiments, the ingredients are supplied either separately or mixed together in unit dosage form, for example, as a dry lyophilized powder or water free concentrate in a hermetically sealed container such as an ampoule or sachette indicating the quantity of active agent. Where an activating agent is to be administered by infusion, it can be dispensed, for example, with an infusion bottle containing sterile pharmaceutical grade water or saline. Where the activating agent is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compounds. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard carriers such as mannitol, lactose, starch, magnesium stearate, sodium saccharine, cellulose, magnesium carbonate, etc. Such carriers can be of pharmaceutical grade.

The compositions can be intended for topical administration, in which case the carrier can be in the form of a solution, emulsion, ointment or gel base. The base, for example, can comprise one or more of the following: petrolatum, lanolin, polyethylene glycols, beeswax, mineral oil, diluents such as water and alcohol, and emulsifiers and stabilizers. Thickening agents can be present in a composition for topical administration. If intended for transdermal administration, the composition can be in the form of a transdermal patch or an iontophoresis device. Topical formulations can comprise a concentration of an activating agent from about 0.1% to about 10% w/v (weight per unit volume of composition).

The composition can be intended for rectal administration, in the form, e.g., of a suppository which will melt in the rectum and release the activating agent. The composition for rectal administration can contain an oleaginous base as a suitable nonirritating excipient. Such bases include, without limitation, lanolin, cocoa butter and polyethylene glycol.

The composition can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The compositions can consist of gaseous dosage units, e.g., it can be in the form of an aerosol. The term aerosol is used to denote a variety of systems ranging from those of colloidal nature to systems consisting of pressurized packages. Delivery can be by a liquefied or compressed gas or by a suitable pump system that dispenses the active ingredients. Aerosols of the activating agent can be delivered in single phase, biphasic, or tri-phasic systems in order to deliver the activating agent(s). Delivery of the aerosol includes the necessary container, activators, valves, sub containers, Spacers and the like, which together can form a kit. Aerosols can be determined by one skilled in the art, without undue experimentation.

Whether in solid, liquid or gaseous form, the compositions of embodiments described herein can comprise a pharmacological agent used in the treatment, prevention, or alleviation of a symptom of cancer or a cell proliferative disease.

The pharmaceutical compositions can be prepared using methodology well known in the pharmaceutical art. For example, a composition intended to be administered by injection can be prepared by combining an activating agent with water so as to form a solution. A surfactant can be added to facilitate the formation of a homogeneous solution or suspension. Surfactants are compounds that non-covalently interact with an activating agent so as to facilitate dissolution or homogeneous suspension of the activating agent in the aqueous delivery system.

In embodiments, the activating agent is a small molecule. The term “small molecule” can refer to a non-peptidic, non-oligomeric organic compound either synthesized in the laboratory or found in nature. Small molecules can refer to compounds that are “natural product-like”, however, the term “small molecule” is not limited to “natural product-like” compounds. Rather, a small molecule is typically characterized in that it possesses one or more of the following characteristics including having several carbon-carbon bonds, having multiple stereocenters, having multiple functional groups, having at least two different types of functional groups, and having a molecular weight of less than 1500, although this characterization is not intended to be limiting for the purposes of embodiments described herein.

The term small molecule scaffold can to a chemical compound having at least one site for functionalization. In an embodiment, the small molecule scaffold can have a multitude of sites for functionalization. These functionalization sites can be protected or masked as can be appreciated by one of skill in this art. The sites can also be found on an underlying ring structure or backbone.

In embodiments, the activating agent is a cytokine. The term “cytokine” can refer to a molecule, such as a protein, that is released by one cell population which act as an intracellular mediator for the same cell population (autocrine) or another cell population (paracrine). Examples of such cytokines are lymphokines, monokines, and traditional polypeptide hormones. Some cytokines are growth hormone such as human growth hormone, N-methionyl human growth hormone, and bovine growth hormone; parathyroid hormone; thyroxine; insulin; proinsulin; relaxin; prorelaxin; glycoprotein hormones such follicle stimulating hormone (FSH), thyroid stimulating hormone (TSH), luteinizing hormone (LH); hepatic growth factor; fibroblast growth factor; prolactin; placental lactogen; tumor necrosis factor-α and beta; Mueller tube inhibiting substance; mouse gonadotropin-associated peptide; inhibin; activin; vascular endothelial growth factor (VEGF); integrin; thrombopoietin (TPO); nerve growth factors such as NGF-beta; platelet-derived growth factor (PDGF); transforming growth factor (TGF), e.g. TG-.alpha. and TGF-beta; insulin-like growth factor (IGF), e.g. IGF-I and IGF-II; erythropoietin (EPO); osteoinductive factors; interferons such as interferon-.alpha.,-beta and -gamma; colony stimulating factors (CSF), such as macrophages-CSF (M-CSF); granulocyte-macrophage-CSF (GM-CSF); and granulocyte-CSF (G-CSF); interleukin (IL), such IL-1, IL-1a, IL-2, IL-3, IL-4, IL-5, IL-6, IL-7, IL-8, IL9, IL-11, IL-12; and other polypeptide factors including LIF and kit ligand (KL, also known as steel factor) is included. In embodiments, the activating agent is a biologic. A “biologic” or “biological agent” can refer to any pharmaceutically active agent made from living organisms and/or their products which is intended for use as a therapeutic. In one embodiment of Embodiments described herein, biologic agents include, but are not limited to e.g., antibodies, nucleic acid molecules (polynucleotides), e.g., antisense nucleic acid molecules, polypeptides or proteins.

In embodiments, the activating agent is a polynucleotide. A “polynucleotide” can encompass a singular “polynucleotide” as well as plural “polynucleotides”. A “polynucleotide” can refer to chain of nucleotides, which can be a nucleic acid, nucleic acid sequence, oligonucleotide, nucleotide, or any fragment thereof. It can be DNA or RNA of genomic DNA, mRNA, cDNA, siRNA, or synthetic origin, double-stranded or single-stranded, and combined with carbohydrate, lipids, protein or other materials to perform a such as activity or form a useful composition.

In embodiments, the activating agent is a polypeptide. A “polypeptide” can encompass a singular “polypeptide” as well as plural “polypeptides,” and can refer to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide can be replaced with another amino acid residue from the same side chain family. In another embodiment, a string of amino acids can be replaced with a structurally similar string that differs in order and/or composition of side chain family members.

In an embodiment, the activating agent is an antibody, an antibody-fragment, or a derivative thereof. As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment”, as used herein, can refer to a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)2, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” can refer to a fusion protein of the variable regions of the heavy (VH) and light chains (VL) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. (See Huston et al. (1988) Proc Nat Acad Sci USA 85(16):5879-5883). In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases in order to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG1, IgG2, IgG3 and IgG4 and others. The immunoglobulin subclasses (isotypes) e.g., IgG1, IgG2, IgG3, IgG4, IgG5, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions largely adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. One example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

As used herein, the term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants usually consist of chemically active surface groupings of molecules such as amino acids or sugar side chains and usually have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3).

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the equilibrium binding constant (KD) of the interaction, wherein a smaller KD represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kon) and the “off rate constant” (Koff) can be determined by calculation of the concentrations and the actual rates of association and dissociation. (See Nature 361:186-87 (1993)). The ratio of Koff/Kon enables the cancellation of all parameters not related to affinity, and is equal to the equilibrium binding constant, KD. (See, Davies et al. (1990) Annual Rev Biochem 59:439-473). An antibody of embodiments described herein can specifically bind to a PD-1 epitope when the equilibrium binding constant (KD) is ≤10 μM, ≤10 nM, ≤10 pM, or ≤100 pM to about 1 pM, as measured by kinetic assays such as radioligand binding assays or similar assays known to those skilled in the art, such as BIAcore. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it can bind to a random, unrelated epitope.

Various procedures known within the art can be used for the production of poly clonal or monoclonal antibodies directed against a protein of Embodiments described herein, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, N.Y., incorporated herein by reference).

Antibodies can be purified by well-known techniques, such as affinity chromatography using protein A or protein G, which provide primarily the IgG fraction of immune serum. Subsequently, or alternatively, the specific antigen, which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site that can immunoreact with a particular epitope of the antigen characterized by a unique binding affinity for it.

Monoclonal antibodies can include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, that exhibit the desired biological activity (see, e.g., U.S. Pat. No. 4,816,567; Morrison et al., 1984, Proc. Natl. Acad. Sci. USA 81:6851-6855). For example, a chimeric antibody can be derived from the variable region from a mouse antibody and the constant region from a human antibody.

“Humanized” forms of non-human (e.g., rodent) antibodies can refer to chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., 1986, Nature 321:522-525; Riechmann et al., 1988, Nature 332:323-329; and Presta, 1992, Curr. Op. Struct. Biol. 2:593-596.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or can produce antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are desired, or spleen cells or lymph node cells can be used if non-human mammalian sources are desired. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, particularly myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines that are useful are those that fuse efficiently, support stable high-level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, Calif.) and the American Type Culture Collection (Manassas, Va.). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of Embodiments described herein can be readily isolated and sequenced using conventional procedures (e.g., by using oligonucleotide probes that can specifically bind to genes encoding the heavy and light chains of murine antibodies). The hybridoma cells of Embodiments described herein can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of Embodiments described herein, or can be substituted for the variable domains of one antigen-combining site of an antibody of Embodiments described herein to create a chimeric bivalent antibody.

Fully human antibodies, for example, are antibody molecules in which the entire sequence of both the light chain and the heavy chain, including the CDRs, arise from human genes. Such antibodies are termed “human antibodies” or “fully human antibodies”. “Humanized antibodies” can be antibodies from non-human species whose light chain and heavy chain protein sequences have been modified to increase their similarity to antibody variants produced in humans. Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, such as improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) For example, the non-human part of the antibody (such as the CDR(s) of a light chain and/or heavy chain) can bind to the target antigen. A humanized monoclonal antibody can also be referred to a “human monoclonal antibody” herein.

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (commonly rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20).

Human monoclonal antibodies, such as fully human and humanized antibodies, can be prepared by using trioma technique; the human B-cell hybridoma technique (see Kozbor, et al, 1983 Immunol Today 4: 72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, antibodies can also be produced using other techniques, including phage display libraries. (See Hoogenboom and Winter, J. Mol. Biol, 227:381 (1991); Marks et al., J. Mol. Biol, 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al, Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies can additionally be produced using transgenic nonhuman animals which are modified so as to produce fully human antibodies rather than the animal's endogenous antibodies in response to challenge by an antigen. (See, PCT publication no. WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. A non-limiting example of such a nonhuman animal is a mouse, and is termed the Xenomouse™ as disclosed in PCT publication nos. WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules. Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, N.Y.) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99/53049.

The antibody of interest can also be expressed by a vector containing a DNA segment encoding the single chain antibody described above. Vectors include, but are not limited to, chemical conjugates such as described in WO 93/64701, which has targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used and include Moloney murine leukemia viruses. DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87: 1149 (1990), Adenovirus Vectors (see LeGal LaSalle et al, Science, 259:988 (1993); Davidson, et al, Nat. Genet 3:219 (1993); Yang, et al, J. Virol. 69:2004 (1995) and Adeno-associated Virus Vectors (see Kaplitt, M. G. et al, Nat. Genet. 8: 148 (1994).

Pox viral vectors introduce the gene into the cell's cytoplasm. Avipox virus vectors result in only a short-term expression of the nucleic acid. Adenovirus vectors, adeno-associated virus vectors, and herpes simplex virus (HSV) vectors can be used for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaP04 precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91:2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways, for example, to try to bind to and disrupt MICA/B shedding from the tumor cell.

Techniques can be adapted for the production of single-chain antibodies specific to an antigenic protein of Embodiments described herein (See e.g., U.S. Pat. No. 4,946,778). In addition, methods can be adapted for the construction of Fab expression libraries (See e.g., Huse, et al, 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen can be produced by techniques known in the art including, but not limited to: (i) an F(ab′)2 fragment produced by pepsin digestion of an antibody molecule; (ii) an Fab fragment generated by reducing the disulfide bridges of an F(ab′)2 fragment; (iii) an Fab fragment generated by the treatment of the antibody molecule with papain and a reducing agent and (iv) Fv fragments.

Heteroconjugate antibodies are also within the scope of embodiments described herein. Heteroconjugate antibodies are composed of two covalently joined antibodies. Such antibodies can, for example, target immune system cells to unwanted cells (see U.S. Pat. No. 4,676,980), and for treatment of HIV infection (See PCT Publication Nos. WO91/00360; WO92/20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of embodiments described herein can be modified with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176: 1 191-1 195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al, Anti-Cancer Drug Design, 3 219-230 (1989)).

In certain embodiments, an antibody of embodiments described herein can comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Without wishing to be bound by theory, Fc variants with improved affinity for FcRn can to have longer serum half-lives, and such molecules have useful applications in methods of treating mammals where long half-life of the administered antibody is desired, e.g., to treat a chronic disease or disorder. In contrast, Fc variants with decreased FcRn binding affinity can to have shorter halt-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be desired include those applications in which localization to the brain, kidney, and/or liver is desired. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of Embodiments described herein comprise an Fc domain having one or more of the following substitutions: V284E, H285E, N286D, K290E and S304D (EU numbering).

In some embodiments, mutations are introduced to the constant regions of the mAb such that the antibody dependent cell-mediated cytotoxicity (ADCC) activity of the mAb is altered. For example, the mutation can be a LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In other embodiments, antibodies of embodiments described herein for use in the diagnostic and treatment methods described herein have a constant region, e.g., an IgG1 or IgG4 heavy chain constant region, which can be altered to reduce or eliminate glycosylation. For example, an antibody of Embodiments described herein can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In an embodiment, the antibody comprises an Fc variant with an amino acid substitution at amino acid position 228 or 299 (EU numbering). In other embodiments, the antibody comprises an IgG1 or IgG4 constant region comprising an S228P and a T299A mutation (EU numbering).

Exemplary amino acid substitutions which confer reduced or altered glycosylation are described in PCT Publication No, WO05/018572, which is incorporated by reference herein in its entirety. In some embodiments, the antibodies of Embodiments described herein, or fragments thereof, are modified to eliminate glycosylation. Such antibodies, or fragments thereof, can be referred to as “agly” antibodies, or fragments thereof, (e.g. “agly” antibodies). While not wishing to be bound by theory “agly” antibodies, or fragments thereof, can have an improved safety and stability profile in vivo. Exemplary agly antibodies, or fragments thereof, comprise an aglycosylated Fc region of an IgG4 antibody which is devoid of Fc-effector function thereby eliminating the potential for Fc mediated toxicity to the normal vital tissues and cells that express PD-1. In yet other embodiments, antibodies of Embodiments described herein, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

Embodiments described herein also is directed to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See PCT Publication No. WO94/11026, and U.S. Pat. No. 5,736,137).

Those of ordinary skill in the art understand that a large variety of moieties can be coupled to the resultant antibodies or to other molecules of Embodiments described herein. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved either by direct condensation of existing side chains or by the incorporation of external bridging molecules. Many bivalent or polyvalent linking agents are useful in coupling protein molecules, such as the antibodies of embodiments described herein, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133: 1335-2549 (1984); Jansen et al., Immunological Reviews 62: 185-216 (1982); and Vitetta et al, Science 238: 1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of Embodiments described herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (-hydroxysulfo-succinimide: Pierce Chem. Co., Cat. #24510) conjugated to EDC.

The linkers described herein can contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, for example, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al, Proc. Natl. Acad. Sci. USA, 82: 3688 (1985); Hwang et al, Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of embodiments described herein can be conjugated to the liposomes as described in Martin et al, J. Biol. Chem., 257: 286-288 (1982) via a disulfide-interchange reaction.

Aspects of embodiments described herein comprise isolated monoclonal antibodies, such as those specific against MICA/B. The term “isolated” as used herein with respect to cells, nucleic acids, such as DNA or RNA, refers to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and not be found in the natural state. “Isolated” can also refer to cells or polypeptides which are isolated from other cellular proteins or tissues. Isolated polypeptides can include both purified and recombinant polypeptides.

An “isolated molecule” (e.g., an antibody) is one which has been identified and separated and/or recovered from a component of its natural environment. Contaminant components of its natural environment are materials which can interfere with diagnostic or therapeutic uses for the molecule, and can include enzymes, hormones, and other proteinaceous or nonproteinaceous solutes. In some embodiments, the molecule will be purified (1) to greater than 95% by weight of molecule as determined by the Lowry method, or to more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. An “isolated molecule” (e.g., an antibody) includes the molecule in situ within recombinant cells since at least one component of the molecule's natural environment will not be present. Ordinarily, however, an isolated molecule will be prepared by at least one purification step.

As described herein, the antibodies or activating agents of embodiments described herein (also referred to herein as “active compounds”), and derivatives, fragments, analogs and homologs thereof, can be incorporated into pharmaceutical compositions suitable for administration. Such pharmaceutical compositions can comprise the antibody or agent and a pharmaceutically acceptable carrier. As described herein in detail, the term “pharmaceutically acceptable carrier” can include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of embodiments described herein is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents can be included, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients described herein, as required, followed by filtered sterilization. For example, dispersions are prepared by incorporating the active compound into a sterile vehicle that contains a basic dispersion medium and the required other ingredients from those described herein. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of Embodiments described herein are dictated by and directly dependent on the unique characteristics of the active compound and the therapeutic effect to be achieved, and the limitations inherent in the art of compounding such an active compound for the treatment of individuals.

The pharmaceutical compositions can be included in a container, pack, or dispenser together with instructions for administration.

Compositions as described herein, such as those containing one or more activating agents, can be administered in combination with one or more additional prophylactic or therapeutic agents, or one or more additional therapeutic or prophylactic regimens. Thus, the term “combination therapy” can refer to a therapeutic regimen comprising at least one activating agent and at least one or more additional prophylactic or therapeutic agents or regimens.

In one embodiment the additional agent, such as an additional chemotherapeutic agent, is that to which treatment of the cancer has not been found to be refractory. In another embodiment, the additional agent is that to which the treatment of cancer has been found to be refractory. The composition can be administered to a patient who has also undergone surgery as treatment for the cancer. In one embodiment, the additional method of treatment is radiation therapy.

In a specific embodiment, the activating agent is administered concurrently with the additional agent, such as a chemotherapeutic agent, or with radiation therapy. In another specific embodiment, the additional agent or radiation therapy is administered prior or subsequent to administration of the composition, in one aspect at least an hour, five hours, 12 hours, a day, a week, a month, in further aspects several months (e.g., up to three months), prior or subsequent to administration of a composition.

In embodiments, the additional therapeutic and/or prophylactic agent comprises a radiotherapeutic agent/radiotherapy, a polynucleotide, a polypeptide, a small molecule, an antibody, a genetically engineered cell, radiation, or any combination thereof.

For example, the phrase a “radiotherapeutic agent” can refer to the use of electromagnetic or particulate radiation in the treatment of neoplasia. Examples of radiotherapeutic agents are provided in, but not limited to, radiation therapy and is known in the art (Hellman, Principles of Radiation Therapy, Cancer, in Principles and Practice of is Oncology, 248-75 (Devita et al., ed., 4 edit., volume 1, 1993).

For example, the additional agent can comprise a small molecule. In embodiments, the small molecule comprises an HDAC inhibitor, such as panobinostat. Panobinostat is a type of drug called a histone deacetylase (HDAC) inhibitor. Panobinostat is a non-selective HDAC inhibitor that inhibits multiple histone deacetylase enzymes which leads to apoptosis of malignant cells via multiple pathways. The skilled artisan will recognize that any one of a number of HDAC inhibitors, including those that are FDA approved or in clinical trials, can be utilized in embodiments described herein. For example, HDAC inhibitors vorinostat, romidepsin, and belinostat have been approved for some T-cell lymphoma and panobinostat for multiple myeloma. See, for example, Eckschlager, Tomas, et al. “Histone deacetylase inhibitors as anticancer drugs.” International journal of molecular sciences 18.7 (2017): 1414. For example, the HDAC inhibitor can be a hydroxamic acid, a short chain fatty acid, a benzamide, a cyclic tetrapeptide, or a sirtuin inhibitor.

In other embodiments, the small molecule can comprise a proteasome inhibitor. Proteasomes are protease complexes which are responsible for degrading endogenous proteins. Proteins to be destroyed are recognized by proteasomes because of the presence of ubiquitin conjugated to the targeted protein. The ubiquitin-proteasome pathway plays an essential role in regulating the intracellular concentration of specific proteins, thereby maintaining homeostasis within cells. Proteasome inhibitors prevent this targeted decomposition of protein, which can affect multiple signaling cascades within the cell. For example, the proteasome inhibitor can be Bortezomib (Velcade), Carfilzomib (Kyprolis) or Ixazomib (Ninlaro).

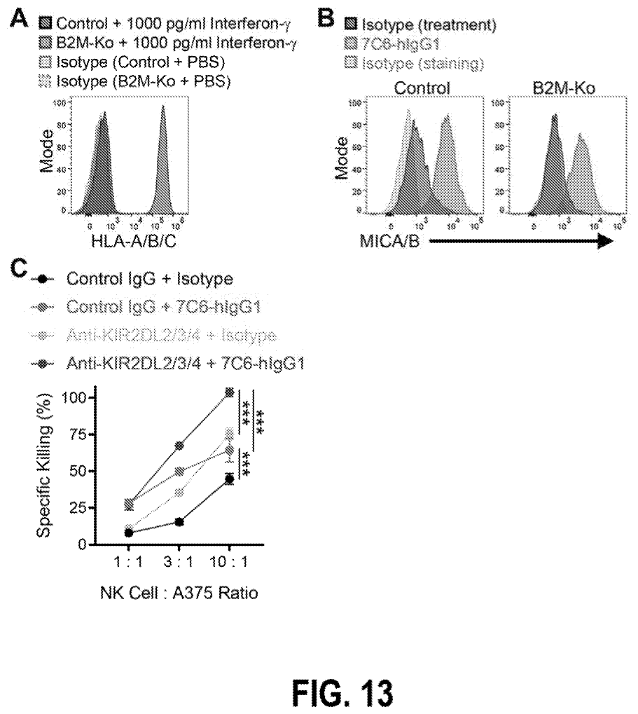

In embodiments, the additional agent can be a polypeptide such as an antibody. In embodiments, the antibody can be an antibody specific for PD1 (anti-PD1 antibody), PDL1 (anti-PDL1 antibody) or CTLA4 (anti-CTLA4 antibody). In other embodiments, the additional therapeutic agent is an antibody that binds to an inhibitory receptor on NK cells, such as KIR, TIGIT, NKG2A and CD161 antibodies. In other embodiments, the antibody can be to CD160, CD96, or TIM-3. Referring to FIG. 13, the antibody can be an anti-KIR2DL2/3/4 antibody.

In embodiments, the additional agent can be a chemotherapeutic agent. A “chemotherapeutic agent” can refer to a chemical compound useful in the treatment of cancer. Chemotherapeutic agents that can be administered with the compositions described herein include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

Examples of chemotherapeutic agents also include alkylating agents such as thiotepa and CYTOXAN® cyclosphosphamide; alkyl sulfonates such as busulfan, improsulfan, piposulfan and treosulfan; decarbazine; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (such as cryptophycin 1 and cryptophycin 8); a dolastatin; duocarmycin (including the synthetic analogues, KW-2189 and CBT-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide or uracil mustard; nitrosureas such as carmustine, chlorozotocin, fotemustine, lomustine, nimustine, and ranimnustine; bisphosphonates such as clodronate; antibiotics such as the enediyne antibiotics (e.g., calicheamicin, especially calicheamicin gamma1I and calicheamicin omegaI1 (see, e.g., Agnew, 1994, Chem. Intl. Ed. Engl. 33: 183-186) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antibiotic chromophores, aclacinomysins, actinomycin, authramycin, azaserine, bleomycins (e.g., bleomycin A2, bleomycin B2 and peplomycin), cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, tiazofurin, ribavarin, EICAR, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; a folic acid replenisher such as folinic acid (leucovorin); aceglatone; anti-folate anti-neoplastic agents such as ALIMTA®, LY231514 pemetrexed, dihydrofolate reductase inhibitors such as methotrexate and trimetrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEXRM, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; deferoxamine; lentinan; lonidainine; a maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; cytosine arabinoside (“Ara-C”); cyclophosphamide; thiotepa; taxoids and taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); epipodophyllins such as etoposide (VP-16), teniposide, tepotecan, 9-aminocamptothecin, camptothecin and crisnatol; ifosfamide; mitoxantrone; vinca alkaloids such as vincristine (ONCOVIN®), vindesine, vinca alkaloid, and vinorelbine (NAVELBINE®); novantrone; edatrexate; daunomycin; aminopterin; xeloda; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Cytokines that can be administered with the compositions include, but are not limited to, IL-2, IL-3, IL-4, IL-5, IL-6, IL-7, IL-10, IL-12, IL-13, IL-15, IL-18, anti-CD40, CD40L, and TNF-α.

In some embodiments, the compositions described herein can be administered in combination with other immunotherapeutic agents. Non-limiting examples of immunotherapeutic agents include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8.

In embodiments, the additional agent can be administered over a series of sessions. Any one or a combination of the additional agents listed herein can be administered. With respect to radiation, any radiation therapy protocol can be used depending upon the type of cancer to be treated. For example, but not by way of limitation, X-ray radiation can be administered; such as, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

Additionally, compositions and methods of treatment of cancer described herein are provided as an alternative to standard anti-cancer regimens, such as chemotherapy or radiation therapy where the chemotherapy or the radiation therapy has proven or can prove too toxic, e.g., results in unacceptable or unbearable side effects, for the patient being treated. The patient being treated can, optionally, be treated with another cancer treatment such as surgery, radiation therapy or chemotherapy, depending on which treatment is found to be acceptable or bearable.

The activating agent also can be used in an in vitro or ex vivo fashion, such as for the treatment of certain cancers, including, but not limited to, leukemias and lymphomas, such treatment involving autologous stem cell transplants.

Methods for treating drug resistant cancer or cancer resistant to cytotoxic T cells include administering to a patient in need thereof an effective amount of at least one activating agent and optionally another therapeutic agent that is an anti-cancer agent. Suitable anticancer agents include those described herein, and include but are not limited to, methotrexate, taxol, L-asparaginase, mercaptopurine, thioguanine, hydroxyurea, cytarabine, cyclophosphamide, ifosfamide, nitrosoureas, cisplatin, carboplatin, mitomycin C, dacarbazine, procarbizine, topotecan, nitrogen mustards, cytoxan, etoposide, 5-fluorouracil, floxuridine, doxifluridine, and ratitrexed, BCNU, irinotecan, a camptothecin, bleomycin, doxorubicin, idarubicin, daunorubicin, dactinomycin, plicamycin, mitoxantrone, asparaginase, vinblastine, vincristine, vinorelbine, paclitaxel, and docetaxel.

Also included are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, megestrol, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, leuprolide acetate, and goserelin; as well as troxacitabine (a 1,3-dioxolane nucleoside cytosine analog); antisense oligonucleotides, such as those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; vitamin DA analogs such as EB 1089, CD 1093 and KH 1060; and pharmaceutically acceptable salts, acids or derivatives of any of the above.

In additional embodiments, the additional therapeutic agent can be a photodynamic agent such as vertoporfin (BPD-MA), phthalocyanine, photosensitizer Pc4, demethoxy-hypocrellin A (2BA-2-DMHA); a cytokine such as Interferon-α, Interferon-γ or tumor necrosis factor; Gemcitabine, Velcade™ (bortezomib), Revlamid™ (lenalidomide) or Thalamid; Lovastatin; 1-methyl-4-phenylpyridinium ion; staurosporine; an actinomycin such as Actinomycin D or dactinomycin; an Anthracyclines such as daunorubicin, doxorubicin (adriamycin), idarubicin, epirubicin, pirarubicin, zorubicin and mtoxantrone; and MDR inhibitors such as verapamil and a Ca2+ATPase inhibitors such as thapsigargin; and pharmaceutically acceptable salts, acids or derivatives of any of the above.

### Other Embodiments

While Embodiments described herein has been described in conjunction with the detailed description thereof, the foregoing description is intended to illustrate and not limit the scope of Embodiments described herein, which is defined by the scope of the appended claims. Other aspects, advantages, and modifications are within the scope of the following claims.

Embodiments described herein will be further described in the following examples, which do not limit the scope of Embodiments described herein described in the claims.

### EXAMPLES

Examples are provided below to facilitate a more complete understanding of embodiments described herein. The following examples illustrate the exemplary modes of making and practicing Embodiments described herein. However, the scope of Embodiments described herein is not limited to specific embodiments disclosed in these Examples, which are for purposes of illustration only, since alternative methods can be utilized to obtain similar results.

### Example 1—Treatment of Cancers Resistant to Checkpoint Blockade Due to Loss of MHC Class I Expression

Checkpoint blockade with antibodies targeting inhibitory receptors on T cells are widely used to treat cancer. Antibodies targeting the PD-1/PD-L1 pathway have been approved for the treatment of a variety of cancers, non-limiting examples of which include melanoma, lung cancer, renal cancer, bladder cancer and Hodgkin's lymphoma. Checkpoint blockade requires cytotoxic T cells as the effector cells of the immune response which kill tumor cells following recognition of tumor-derived peptides bound to MHC class I proteins. Tumors that lose MHC class I expression due to inactivating genetic mutations or epigenetic mechanisms are resistant to checkpoint blockade. However, MHC class I proteins serve as a ligand for inhibitory KIR receptors on NK cells. Loss of this inhibitory signaling is however not sufficient for induction of tumor immunity because activating signals for NK cells are absent or weak.

We have shown that MHC class I deficient tumor cells are efficiently killed in the presence of MICA antibodies which activate NK cells through two major receptors, NKG2D and CD16. Importantly, we demonstrate that a MICA antibody has single-agent activity against treatment resistant cancer cells in vivo. Without wishing to be bound by theory, MHC class I deficient tumor cells can be targeted with monoclonal antibodies that induce NK cell activation through the major activating receptors, including NKG2D and CD16. For example, such monoclonal antibodies can be MICA antibodies or other tumor-targeting antibodies that activate NK cells.

There are currently no immunotherapies for cancers that have lost MHC class I expression. Such cancers are resistant to all immunotherapies that require cytotoxic T cells as the effector mechanism. The clinical need in this area is high. With our approach, NK cells are activated due to signaling through two major NK cell receptors (NKG2D and CD16) which is not opposed by inhibitory signals from KIR receptors that bind MHC class I proteins.

Aspects of embodiments described herein comprise the use of combination therapy with PD-1 or CTLA-4 checkpoint blockade in patients resistant to monotherapy with checkpoint blockade. Only 20% of patients respond to PD-1 blockade in most responsive cancers, so there is a lot of room for improvement.

### Example 2—Harnessing Natural Killer Cells for the Treatment of Tumors Resistant to Cytotoxic T Cells

Abstract

Resistance to cytotoxic T cells is frequently mediated by loss of MHC class I expression or IFNγ signaling in tumor cells, such as mutations of B2M or JAK1 genes. NK cells can target such resistant tumors, but suitable NK cell-based strategies remain to be developed. Aspects of embodiments described herein address this shortcoming. We show that B2M and JAK1 deficient metastases were targeted by NK cells following treatment with a mAb that blocked MICA/B shedding, a frequent evasion mechanism in human cancers. We also performed a single cell analysis of NK cells in human melanoma metastases, including patients who progressed following checkpoint blockade. We identified major transcriptional differences between tumor-infiltrating and circulating NK cells. Also, the gene expression programs of seven tumor-infiltrating NK cell clusters indicate significant specialization, including cytotoxicity and chemokine secretion. NK cell-based immunotherapy therefore provides an opportunity to target tumors with mutations that render them resistant to cytotoxic T cells.

Introduction

Checkpoint blockade with antibodies targeting the programmed cell death protein 1 (PD-1) or cytotoxic T-lymphocyte-associated protein 4 (CTLA-4) inhibitory receptors on T cells can induce durable anti-tumor immunity even in patients with advanced cancer. However, many patients fail to benefit from these therapies due to primary or secondary resistance.¹ Cytotoxic T cells play a central role in the efficacy of checkpoint blockade based on their ability to recognize tumor-derived peptides bound to major histocompatibility complex class I (MHC-I) proteins.² Recognition of such MHC-I—peptide complexes by the T cell receptor (TCR) triggers the release of interferon-γ (IFNγ) by T cells which inhibits tumor cell proliferation and enhances expression of MHC-I proteins on both tumor and dendritic cells.³ Resistance to checkpoint blockade is therefore frequently mediated by loss of MHC-I expression by tumor cells, either by mutation or epigenetic silencing of key genes in the MHC-I (B2M, TAP1, TAP2 and other genes) or IFNγ (JAK1, JAK2) pathways.^{4, 5, 6} A low number or loss of neoantigens also diminishes tumor immunity mediated by cytotoxic T cells.^{7, 8, 9, 10} There are currently no alternative immunotherapies for patients with solid tumors resistant to checkpoint blockade.

Natural Killer (NK) cells recognize tumor cells by molecular mechanisms that differ substantially from those required by cytotoxic T cells. NK cell recognition of tumor cells is mediated by ligands associated with malignant transformation, including DNA damage and cellular stress.¹² Without wishing to be bound by theory, tumors resistant to cytotoxic T cells can respond to NK cell-based immunotherapy approaches. In fact, loss of MHC-I expression by tumor cells render them more sensitive to NK cells because MHC-I proteins serve as ligands for inhibitory NK cell receptors.¹² However, induction of NK cell-mediated tumor immunity can also require effective targeting of immune evasion mechanisms that hinder NK cell-mediated attack of tumor cells. For example, many human cancers express the MHC-I polypeptide-related sequence A (MICA) and MICB (MICA/B) proteins that serve as ligands for the activating NK group 2D (NKG2D) receptor on NK cells and subpopulations of T cells.^{13, 14} However, tumors frequently evade NKG2D receptor-mediated tumor immunity by proteolytic shedding of MICA/B proteins.^{15, 16, 17, 18, 19} We recently developed monoclonal antibodies (mAbs) that bind to the 3 domain of MICA/B, a domain essential for shedding. See Table 1, for example, and WO2018217688A1, which is incorporated by reference herein in its entirety. These mAbs inhibited MICA/B shedding and induced NK cell-mediated tumor immunity. The increased density of MICA/B proteins on tumor cells enhanced NKG2D receptor-mediated activation in NK cells, and the Fc segment of tumor-bound antibodies also activated NK cells through the CD16 Fc receptor. Treatment with such MICA/B antibodies induced a striking shift of tumor-infiltrating NK cells to a highly cytotoxic state.²⁰

Therefore, without wishing to be bound by theory, NK cell-based therapies can treat metastatic lesions resistant to cytotoxic T cells. However, rather little is known about human NK cells that infiltrate solid tumors. We performed a single cell analysis of NK cells infiltrating human melanoma metastases, including metastases from patients whose tumors had progressed following checkpoint blockade. These data identified new NK cell subpopulations and highlighted striking differences between tumor-infiltrating and circulating NK cell populations. Treatment with a MICA/B α3 domain mAb enabled NK cell-mediated immunity against tumors with inactivating mutations in the MHC-I or IFNγ signaling pathways (B2m and Jak1 mutations, respectively). These results will help to guide the development of NK cell-based immunotherapies for human cancers resistant to cytotoxic T cells and the validation thereof.

Results

Single Cell Characterization of NK Cells Infiltrating Human Melanoma Metastases

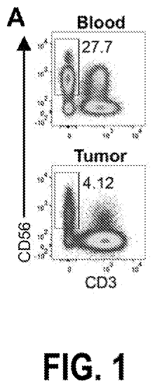

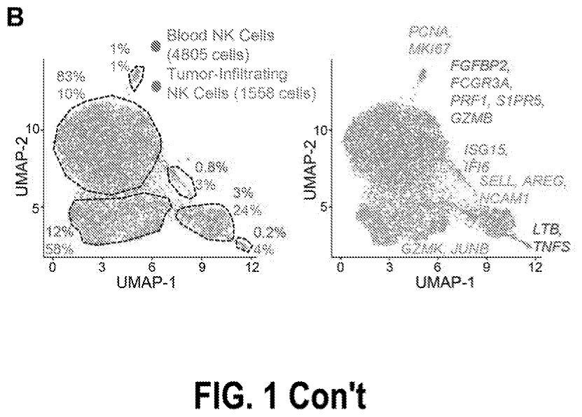

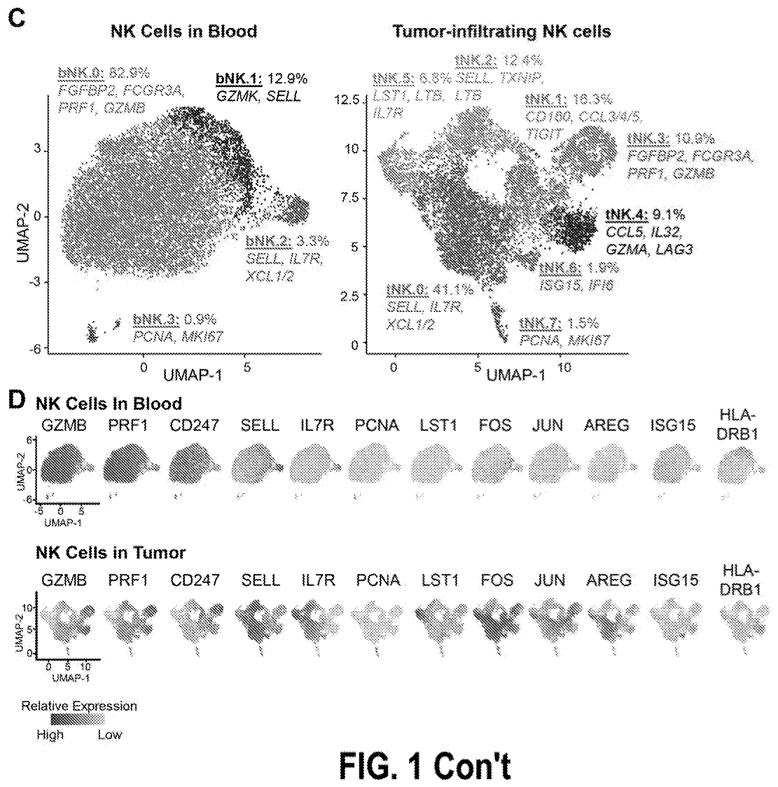

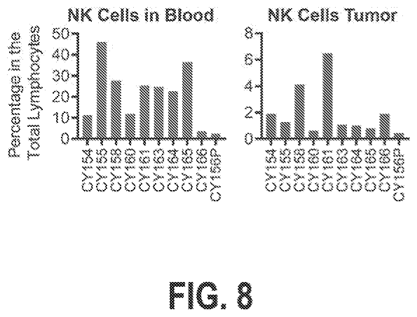

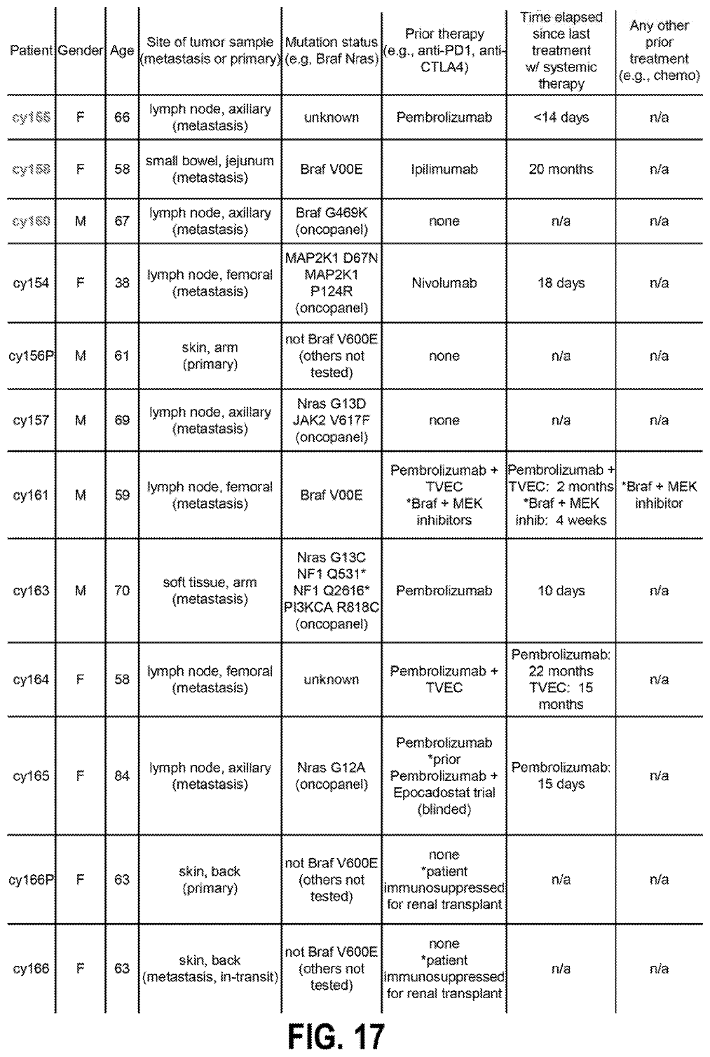

Increasing evidences validates a significant role played by NK cells in the tumor immunity.^{21, 22, 23, 24, 25, 26, 27, 28} However, little is known about the NK cell populations infiltrating human solid tumors. We therefore performed single cell studies of NK cells infiltrating human melanoma metastases that required surgical resection. In the majority of these patients, tumors had progressed following treatment with checkpoint blockade (FIG. 17). NK cells were sorted from tumors and matching blood samples by flow cytometry as CD45⁺ CD56⁺ CD3⁻ CD4⁻ CD8a⁻ CD14⁻ CD15⁻ CD163⁻ viable lymphocyte-size cells. NK cells identified with these markers were present at lower frequencies in tumors compared to blood samples. The NK cell frequency in the total lymphocyte population varied from 2.47% to 46.10% in the blood and 0.46% to 6.48% in the tumor (FIG. 8). For example, NK cells represented 4.12% of total CD45⁺ lymphocytes in the tumor and 27.7% of total CD45⁺ lymphocytes in the blood of patient CY158 (FIG. 1).

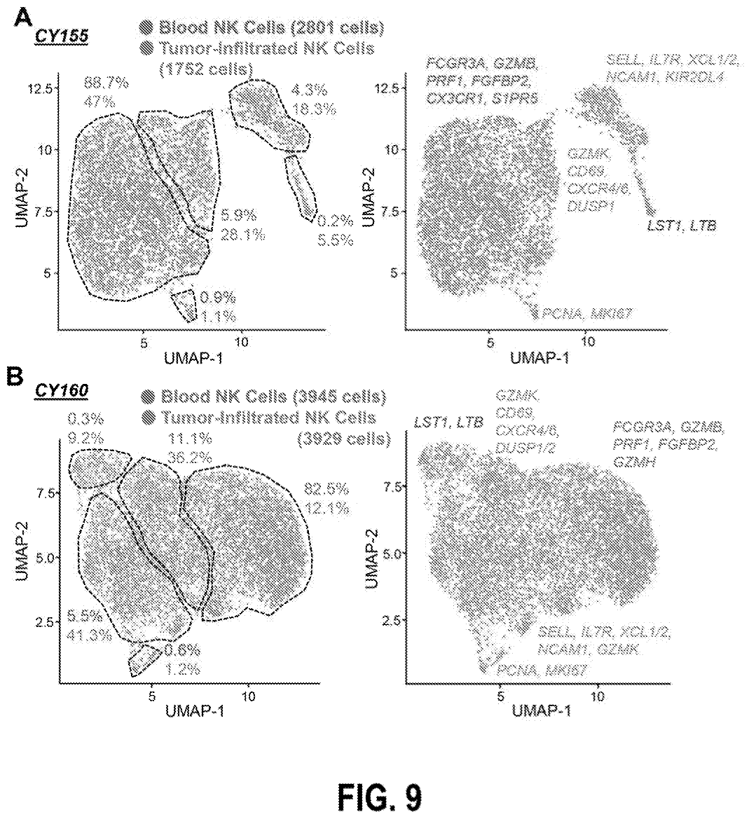

We examined the transcriptome of NK cells isolated from melanoma metastases and matching blood samples from three patients (CY155, CY158, and CY160) by single-cell RNA-seq (scRNA-seq) using the 10× Genomics platform. A non-linear dimensionality reduction technique, uniform manifold approximation and projection (UMAP) was used to visualize NK cell clusters. An integrated analysis of tumor versus blood NK cells from each patient demonstrated major differences in the distribution of NK cells across clusters, indicating that there were substantial differences in the transcriptome of tumor-infiltrating versus circulating NK cells. NK cell clusters with similar gene expression profiles were identified in all three patients; the only exception was a NK cell population characterized by differential expression of interferon-inducible genes (ISG15, IFI6) which only was present in the tumor from patient CY158 (FIG. 1; FIG. 9).

An analysis of blood NK cells from all three patients led to the identification of four blood NK cell clusters (bNK.0-bNK.3). A similar analysis of tumor-infiltrating cells from all three patients resulted in the identification of eight clusters of NK cells (tNK.0-tNK.7) (FIG. 1). Two NK cell clusters identified in tumors shared transcriptional features with blood NK cells but were present at strikingly different frequencies in these locations. The predominant NK cell cluster in tumors (tNK.0, 41.1%) shared a transcriptional signature with a minor NK cell population in blood (bNK.2, 3.3%) (SELL, IL7R, XCL1 and XCL2). The predominant NK cell cluster in blood (bNK.0) shared expression of a number of key genes with cluster tNK.3 in tumors (including FGFBP2, FCGR3A, PRF1, GZMB), but represented a much lower fraction of tumor-infiltrating NK cells (10.9% compared to 82.9% in blood). Also, small proliferating NK cell clusters were present in both blood (bNK.3, 0.9%) as well as tumors (tNK.7, 1.5%), and these cells shared expression of cell cycle genes (including PCNA and MKI67). The other five NK cell clusters identified in melanoma metastases (tNK.1, tNK.2, tNK.4, tNK.5 and tNK.6) were quite distinct in their transcriptional programs from blood NK cell clusters (FIG. 1).

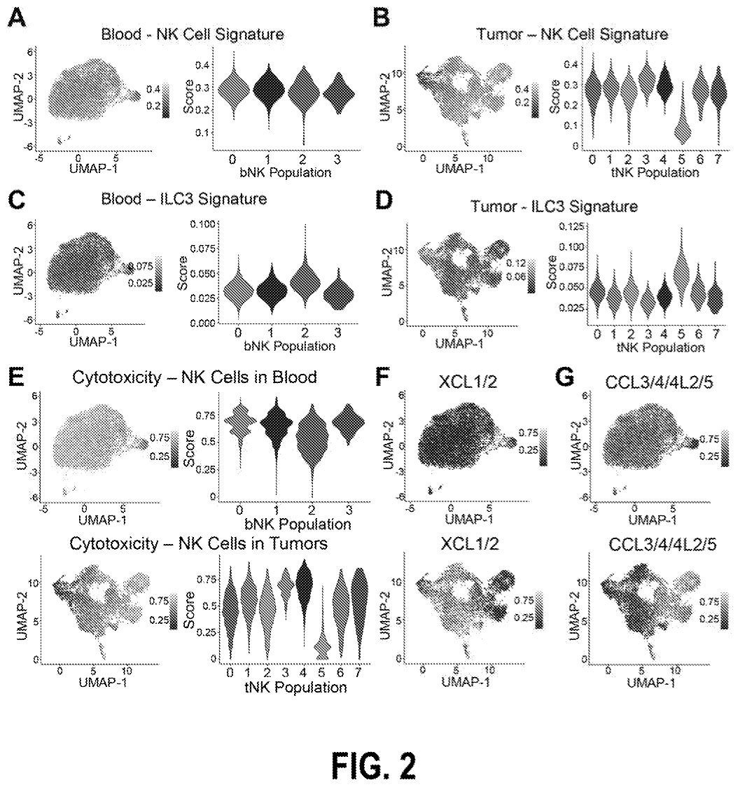

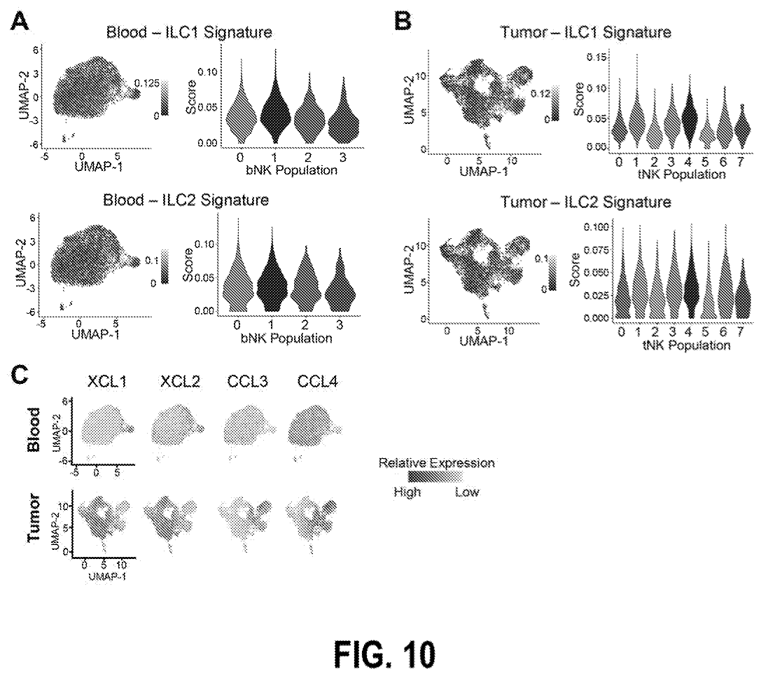

The markers used for isolation of NK cells (FIG. 1) are also expressed by innate lymphoid cells (ILCs) which include the ILC1, ILC2, and ILC3 subpopulations.²⁹ ILCs are known to be tissue-resident cells and it was therefore likely that ILCs were not present in blood samples.³⁰ However, it was important to assess whether some of the NK cell clusters identified in melanoma metastases can represent ILCs. A previous scRNA-seq study had identified genes differentially expressed by NK cells, ILC1, ILC2, and ILC3, and we used these genes to assemble transcriptional signatures for each of these innate lymphocyte populations.³¹ All NK cell clusters in the blood had a strong NK cell gene expression signature but a low score for all three ILC signatures (FIG. 2 & FIG. 10). Furthermore, 7 of 8 NK cell clusters identified in tumors also had a high score for the NK cell gene expression signature (FIG. 2). The exception was the tNK.5 cluster (gold color) which had a high score for the ILC3 gene expression signature but a low score for the NK cell, ILC1 or ILC2 signatures (FIG. 2 & FIG. 10). Finally, none of the tumor or blood NK cell populations had strong ILC1 or ILC2 gene expression signatures (FIG. 10). Therefore, NK cells (7 of 8 clusters, total of 93.2% cells) and ILC3-like cells (one cluster, 6.8% of cells) were identified in human melanoma metastases.

Gene Expression Programs Related to Key NK Cell Functions

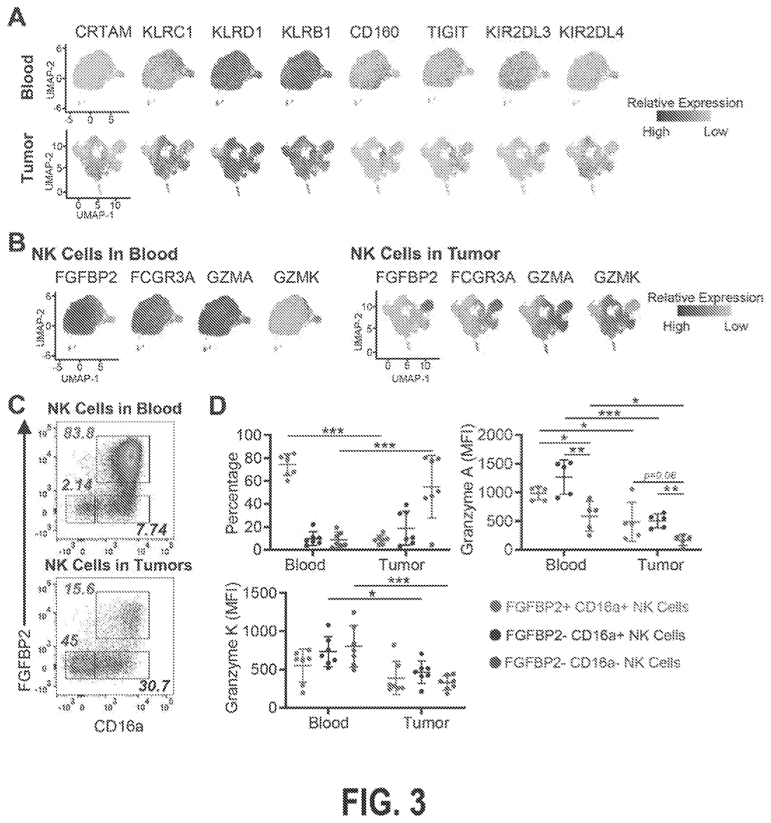

The investigation of NK cells in tumor immunity has primarily focused on their cytotoxic function, but recent studies have highlighted an important role of NK cells in recruitment of dendritic cells that are critical for induction of T cell-mediated tumor immunity.^{24, 26} We used a panel of genes encoding key cytotoxicity proteins to assemble a cytotoxicity gene expression signature (GZMA, GZMB, GZMH, GZMK, GZMM, PRF1, GNLY and NKG7). This cytotoxicity signature was high in most blood NK cells, in particular in clusters bNK.0, bNK.1 and bNK.3. In tumor NK cells, we observed a gradient for this cytotoxicity signature which was the highest in tNK.3 and tNK.4 clusters and at an intermediate level in five other clusters. Only the tNK.5 cluster was negative for this cytotoxicity signature, consistent with designation of these cells as ILC3 (FIG. 2). We also observed different patterns of expression for granzyme genes across NK cell clusters: GZMA expression was high in most blood NK cells, but showed a gradient similar to the cytotoxicity signature in tumor NK cells. Interestingly, GZMK expression showed a distinct pattern: it was low in most blood NK cells but high in many of the tumor NK cell clusters (FIG. 3).

We also observed striking differences in the expression of chemokine genes between tumor and blood NK cells. The chemokines XCL1 and XCL2 (that bind to the XCR1 chemokine receptor) were recently shown to play a critical role in recruiting cross-presenting DCs (cDC1) to tumors.²⁶ Expression of these two chemokine genes was substantially higher in tumor NK cells (clusters tNK.0, tNK.1, tNK.2, tNK.6) compared to blood NK cells (FIG. 2 and FIG. 10). Also, we observed high expression of another set of chemokine genes (CCL3, CCL4, CCL4L2 and CCL5) in many tumor NK cells (clusters tNK.3, tNK.4 and tNK.1) while expression in most blood NK cells was low (FIG. 2 & FIG. 10). These chemokines bind to CCR5 and other chemokine receptors and play a critical role in recruitment of T cells and other immune cells. CCL5 is also known to contribute to the recruitment of cross-presenting DCs.^{26, 32} Thus, tumor-resident NK cells express many chemokine genes that are important for recruitment of DCs, T cells and other immune cell populations. We note that tumor-infiltrating compared to blood NK cells expressed substantially higher levels of FOS and JUN which encode the subunits of the AP-1 transcription factor (FIG. 1). The single cell data also clearly demonstrated functional specialization among tumor NK cell populations in terms of chemokine gene expression: four clusters of tumor NK cells showed high expression of XCL1/XCL2, while a distinct set of tumor NK cell clusters showed high expression of CCL3, CCL4, CCL4L2 and CCL5. These tumor NK cell populations can thus create distinct microenvironments.

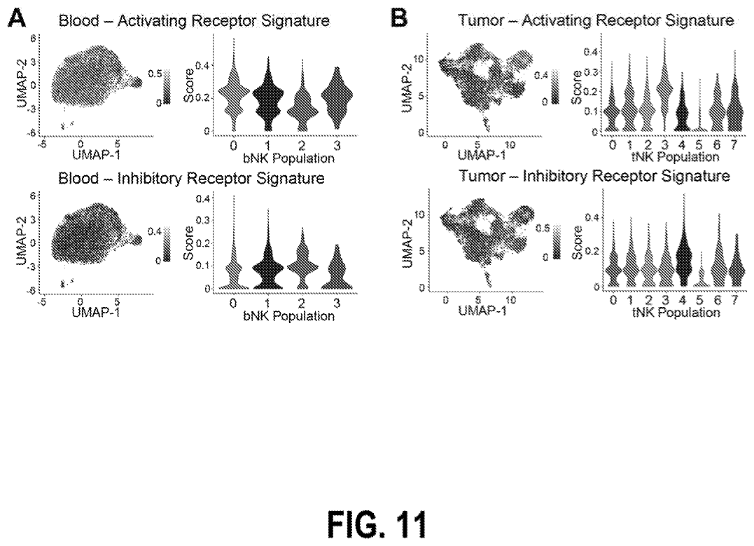

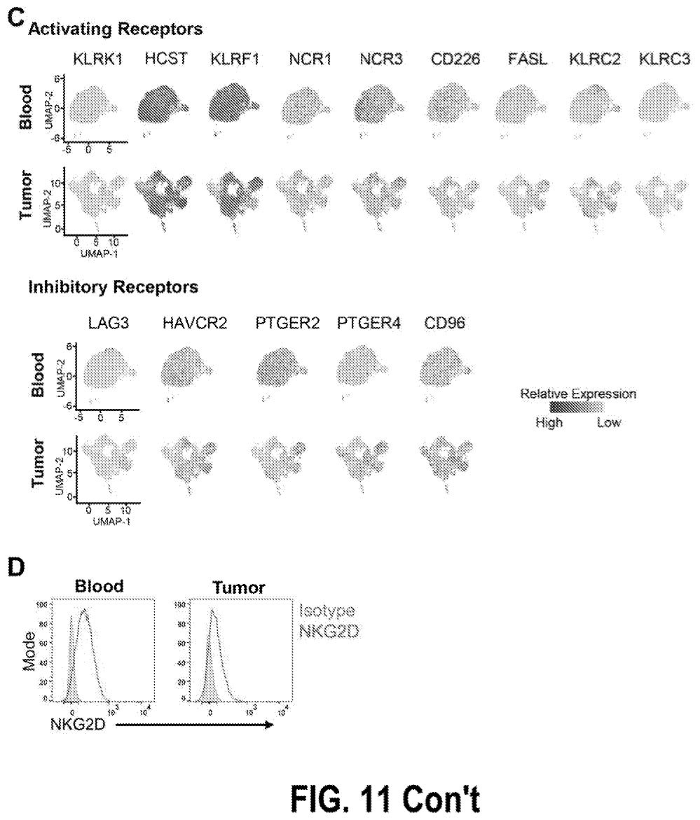

NK cells integrate signals from the extracellular environment through a series of activating and inhibitory receptors (FIG. 11). Among the genes encoding activating receptors, a strong signal was observed for KLRF1 (NKp80 protein) in a large fraction of blood and tumor NK cells (FIG. 11). The AICL gene, which encodes the ligand for NKp80, can be expressed in both hematological malignancies and solid tumors.³³ Signals for other well-established activating NK cell receptors were lower (NCR1, NCR3, CD226 and NKG2D), but is important to note that some mRNAs tend to yield rather weak signals by scRNA-seq even though both mRNA and protein are quite abundant in the relevant cell populations. For example, although KLRK1 mRNA was low in all NK cell populations including blood NK cells (KLRK1 encodes the NKG2D protein), HCST mRNA was high (HCST mRNA encodes DAP10, which is the adaptor molecule for NKG2D). Thus, without wishing to be bound by theory, KLRK1 mRNA was difficult to detect by scRNA-seq, whereas HCST was detected. On the other hand, published reports demonstrated that NKG2D protein can detected on human blood NK cells from melanoma patients, yet at lower levels compared to health donors.^{34, 35} NKG2D expression was lower on tumor-infiltrating compared to blood NK cells (FIG. 11), likely due to ligand-induced downregulation and TGFβ-induced changes in gene expression.³⁶

We also observed interesting expression patterns for receptors with established inhibitory function in NK cells. Tumor-infiltrating NK cells expressed higher levels of the KLRC1 gene (NKG2A protein) than blood NK cells, and the KLRD1 gene (CD94 protein) was highly expressed by most tumor and blood NK cells. This indicates that a large fraction of melanoma-infiltrating NK cells express the inhibitory NKG2A-CD94 receptor that recognizes HLA-E. We also observed a strong signal for the KLRB1 gene (CD161 protein) in both tumor and blood NK cells, and CD161 is known to inhibit NK cell cytotoxicity following binding to CLEC2D on tumor cells and APCs.³⁷ The signals for most other inhibitory receptors were weaker, yet interestingly distinct expression patterns emerged: CD96 was expressed across NK cell clusters, while expression of other receptors was limited to one or a small subset of NK cell clusters (such as CD160 and KIR2DL3). Also, KIR2DL3 and KIR2DL4 showed different expression patterns across NK cell clusters even though they belong to the same family of inhibitory receptor genes (FIG. 3 & FIG. 11).

Validation of scRNA-Seq Data by Flow Cytometry

We used flow cytometry to validate key findings from the scRNA-seq data and extend the analysis to a larger population of melanoma patients. We identified NK cells using well established markers (CD45⁺ CD56⁺ CD3⁻ CD4⁻ CD8a⁻ CD14⁻ CD15⁻ CD163⁻ CD19⁻ viable lymphocyte-size cells), and then used the CD16a and FGFPB2 markers based on the scRNA-seq data to identify key NK cell subpopulations. Of importance, CD16a protein is encoded by the FCGR3A gene. This analysis identified three cell populations: 1) FGFBP2⁺ CD16a⁺ NK cells that corresponded to bNK.0 (the most abundant population in blood) and tNK.3 that expressed key cytotoxicity genes (PRF1 and GZMB); 2) FGFBP2⁻ CD16a⁺ NK cells that can be resolved from the CD16a⁺ population using FGFPB2 as a marker; these cells corresponded to bNK.1 as well as tNK.4 and tNK.1 that expressed FCGR3A but not FGFBP2. 3) FGFBP2⁻ CD16a⁻ NK cells that corresponded to bNK.2 (a small blood subset) and abundant tumor NK cell populations that did not express FGFBP2 and FCGR3A (primarily but not exclusively tNK.0 and tNK.2) (FIG. 1 & FIG. 3).

We used the FGFBP2 and CD16a markers to examine these three NK cell populations in melanoma and matching blood samples from a total of seven patients. The predominant NK cell population in blood samples was positive for both FGFBP2 and CD16a while a large fraction of tumor-infiltrating NK cells was negative for both FGFBP2 and CD16a (FIG. 3), consistent with the scRNA-seq data (FIG. 3). We further examined the expression of granzymes A and K in these NK cell populations. The expression of granzymes A and K was higher in blood compared to tumor NK cells. Also, granzyme A levels were higher in FGFBP2 positive and negative CD16a⁺ blood NK cells compared to FGFBP2⁻ CD16a⁻ blood NK cells (FIG. 3), consistent with the scRNA-seq data (FIG. 3).

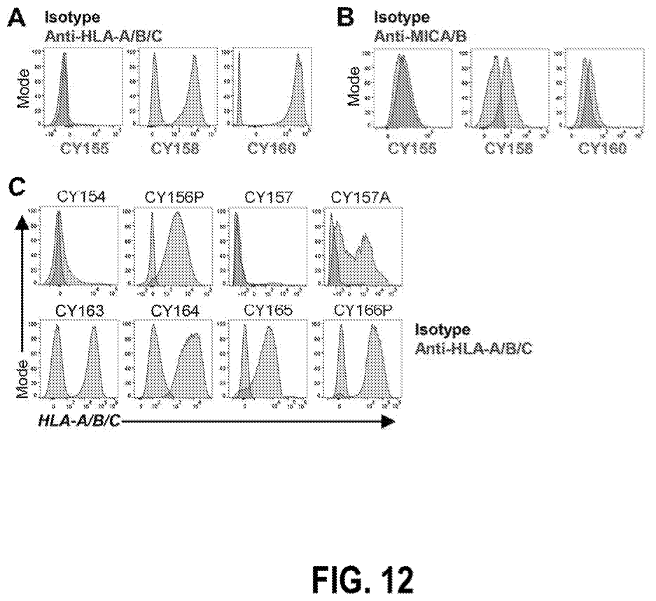

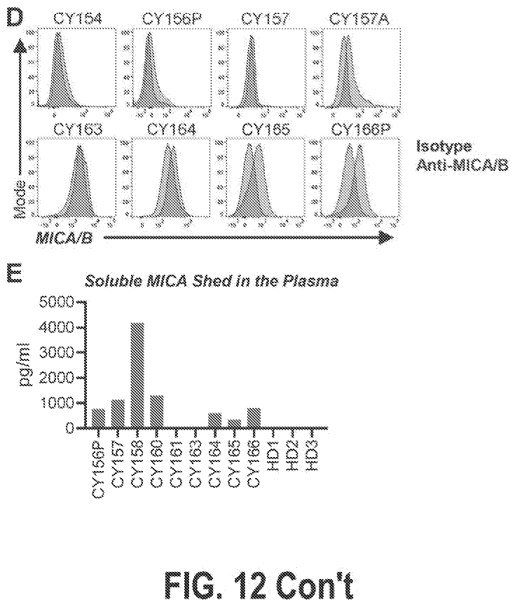

HLA-A/B/C proteins were detected on the surface of melanoma cells from 8 patients but were low or undetectable on tumor cells from three patients (FIG. 12). In particular, one of the tumors studied by scRNA-seq (CY155) had undetectable surface HLA-A/B/C protein, and this patient had progressed following treatment with a PD-1 mAb. Surface MICA/B protein was detected on the surface of melanoma cells in the majority of cases, although at a relatively low level. In 7 of 9 serum samples, shed MICA was detected, consistent with loss of surface MICA from tumor cells by shedding (FIG. 12). Shed MICA was not detected in sera from healthy subjects.

MICA Antibody Enhanced NK Cell-Mediated Killing of Human B2M-Deficient Melanoma Cells

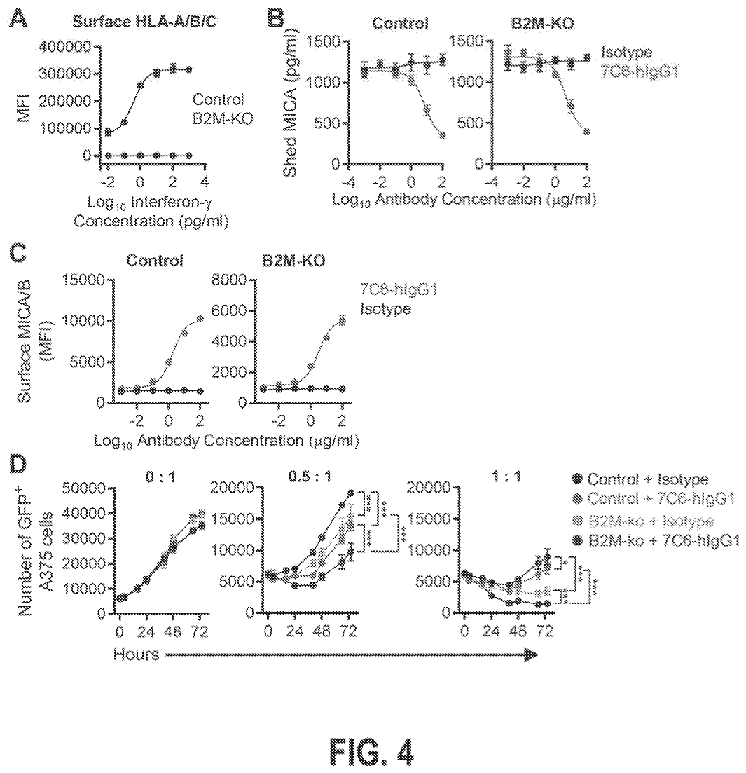

The single cell data demonstrate that NK cells infiltrate human melanoma metastases, including lesions resistant to checkpoint blockade (FIG. 17). We therefore examined whether a MICA/B α3 domain specific antibody can enhance NK cell-mediated immunity against B2M-deficient tumor cells.²⁰ The MICA and MICB genes are part of the MHC locus on human chromosome 6, and the encoded proteins share significant structural similarity with MHC-proteins. B2M-deficiency abrogates T cell-mediated immunity and responsiveness to T cell checkpoint blockade, but MICA/B proteins do not associate with 32 microglobulin or peptides.^{5, 38, 39} We inactivated the B2M gene in human A375 melanoma cells which resulted in a complete loss of MHC-I surface proteins even following stimulation with IFNγ (FIG. 4 & FIG. 13). B2M deficiency did not interfere with the MICA/B pathway, but treatment with a MICA/B α3 domain specific mAb (7C6-hIgG1) inhibited MICA shedding and increased surface levels of MICA/B to a similar extent for control and B2M-edited A375 cells (FIG. 4 & FIG. 13).

NK cells have inhibitory receptors for MHC-I molecules¹², and, without wishing to be bound by theory, B2M-deficient tumor cells can be more sensitive to MICA/B mAb treatment. We studied the kinetics of NK cell-mediated killing of human A375 melanoma cells using an imaging-based system that enabled counting of fluorescent tumor cells in 96-well plates at multiple time points. A major advantage of this technique is that it enables investigation of NK cell—tumor cell interactions at low effector to target ratios that are more relevant to the tumor microenvironment.⁴⁰ This experiment demonstrated that MICA/B mAb treatment (7C6-hIgG1) was substantially more effective against B2M-KO compared to control A375 melanoma cells. Even at a low effector to target ratio (1:1), only a small number of fluorescent B2M-KO melanoma cells remained at late time points (48-72 hours) in the presence of the MICA/B mAb (FIG. 4). KIR2DL2, KIR2DL3, and KIR2DL4 are some of the best characterized inhibitory receptors for MHC-I molecules on human NK cells.¹² Antibody-mediated blockade of those receptors increased NK cell-mediated killing of 7C6-hIgG1-treated A375 melanoma cells (FIG. 13). These experiments demonstrated that loss of MHC class I surface expression rendered human tumor cells more vulnerable to NK cells in the presence of a MICA/B mAb.

A MICA B Antibody Induced NK Cell-Driven Immunity Against Metastases Resistant to Cytotoxic T Cells

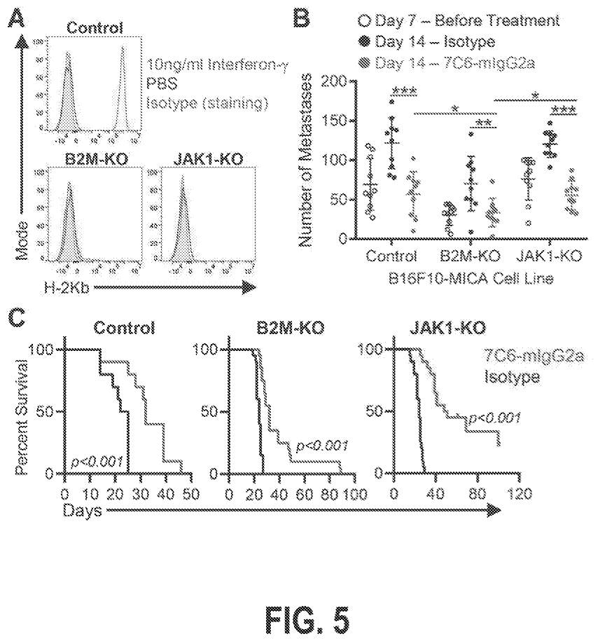

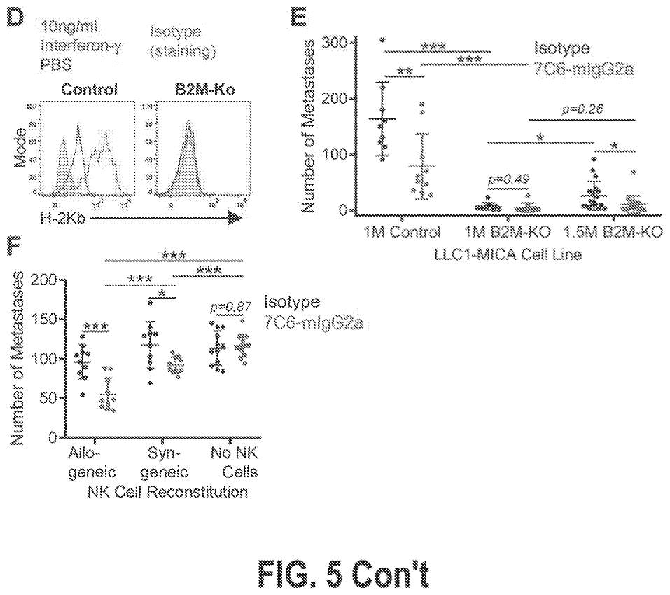

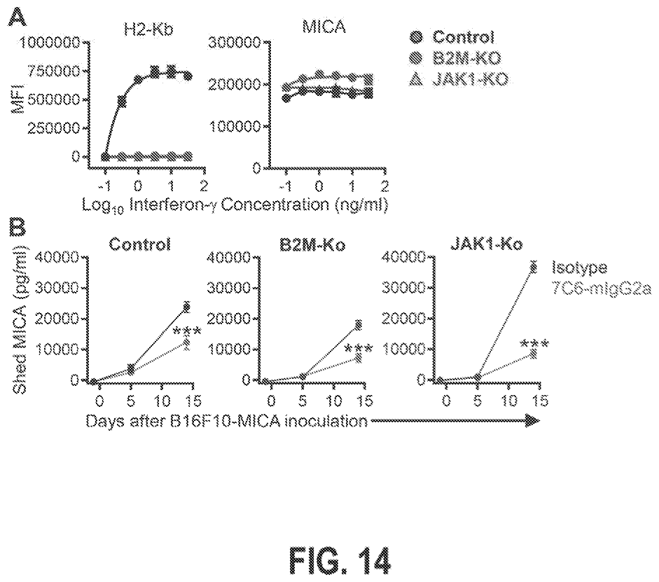

We used two murine models to investigate whether MICA/B mAb treatment can induce NK cell-driven immunity against tumors with inactivating mutations in the MHC-I and IFNγ pathways (B2m and Jak1 mutations, respectively). The Jak1 mutation was of particular interest because IFNγ is secreted by both T cells and NK cells. IFNγ signaling in tumor cells not only enhances expression of many genes of the MHC class I pathway, but also inhibits tumor cell proliferation.³ Therefore, Jak1 mutations can either negatively impact the ability of NK cells to control tumor cell growth or enhance NK cell activation through loss of MHC class I proteins that engage inhibitory receptors on NK cells. B16F10 melanoma and LLC1 lung cancer cell lines were transduced with a lentiviral vector to induce expression of human MICA which is known to bind to the murine NKG2D receptor.²⁰ These murine models had important differences in their pattern of MHC class I expression. B16F10 melanoma cells had very low basal surface level of H-2K^{b} protein, but exposure to IFNγ resulted in a striking increase of H-2K^{b} surface protein (FIG. 5 and FIG. 14). In contrast, LLC1 lung tumor cells had detectable basal levels of H-2K^{b} which was increased by IFNγ treatment (FIG. 5).

We inactivated the B2m or Jak1 genes in B16F10-MICA melanoma cells (FIG. 5 & FIG. 14) and tested the efficacy of MICA/B mAb treatment in a lung metastasis model. Edited tumor cells were injected intravenously, and treatment was initiated on day 7 when established surface lung metastases were detected (as determined by pathological analysis of a subset of mice, labeled as ‘before treatment group’) (FIG. 5). B cell deficient Ighm^{-/-} mice were used as hosts to prevent development of endogenous antibodies against human MICA, as previously reported.²⁰ Treatment with the MICA mAb (7C6-mIgG2a) inhibited the outgrowth of lung metastases by control, B2m-KO and Jak1-KO B16F10-MICA cells (FIG. 5). MICA mAb treatment also reduced plasma levels of shed MICA (FIG. 14). MICA mAb administered on days 1 and 2 relative to B16F10-MICA inoculation significantly increased survival of wild type (WT) mice with control, B2m-KO or Jak1-KO melanoma metastases compared to isotype control mAb treatment (FIG. 5). Surprisingly, there was a greater survival benefit in the JAK1-KO mice when compared to the B2M-KO mouse. This finding was surprising because it was not known whether JAK1-KO mice would respond (because NK cells also secrete gamma interferon). As shown herein, NK cells are still active against tumors with loss mutations in gamma interferon pathway. We also examined the efficacy of MICA/B antibody treatment in the LLC1-MICA tumor model. Of note, control LLC1 cells express MHC-I at baseline and treatment with IFNγ increases MHC-I surface protein levels, whereas B2m-KO LLC1 cells have no MHC-I expression even if treated with IFNγ (FIG. 5). Tumor cells were injected intravenously into WT mice, and mAb treatment was initiated on day 2. MICA mAb treatment reduced the number of lung metastases formed by control LLC1-MICA tumor cells. Inactivation of the B2m gene reduced the number of lung metastases compared to control LLC1-MICA cells to almost undetectable levels. We therefore increased the number of inoculated tumor cells by 50% which resulted in formation of lung metastases by B2m-KO LLC1-MICA cells. Under these experimental conditions, we observed a significant reduction in the number of B2m-KO LLC1-MICA metastases following treatment with 7C6-mIgG2a compared to isotype control mAb (FIG. 5). We further investigated the role of NK cells and inhibitory receptors for MHC-I proteins using an adoptive transfer model. Rag2^{-/-} Il2rg^{-/-} KO mice were reconstituted with either syngeneic (from C57BL/6 mice) or allogeneic (from CB6F1 mice) NK cells. Both syngeneic and allogeneic NK cells significantly reduced the number of lung metastases formed by LLC1-MICA tumor cells when mice were treated with MICA/B versus isotype control mAb. Also, MICA/B antibody treatment was more effective when allogeneic NK cells were transferred (FIG. 5). Allogeneic NK cells are not inhibited by MHC class I proteins on tumor cells (such as LLC1-MICA cells), as reported previously.⁴¹ Without wishing to be bound by theory, the data validates that engagement of MHC-I proteins by inhibitory receptors on NK cells reduces the efficacy of anti-tumor immunity induced by the MICA/B mAb.

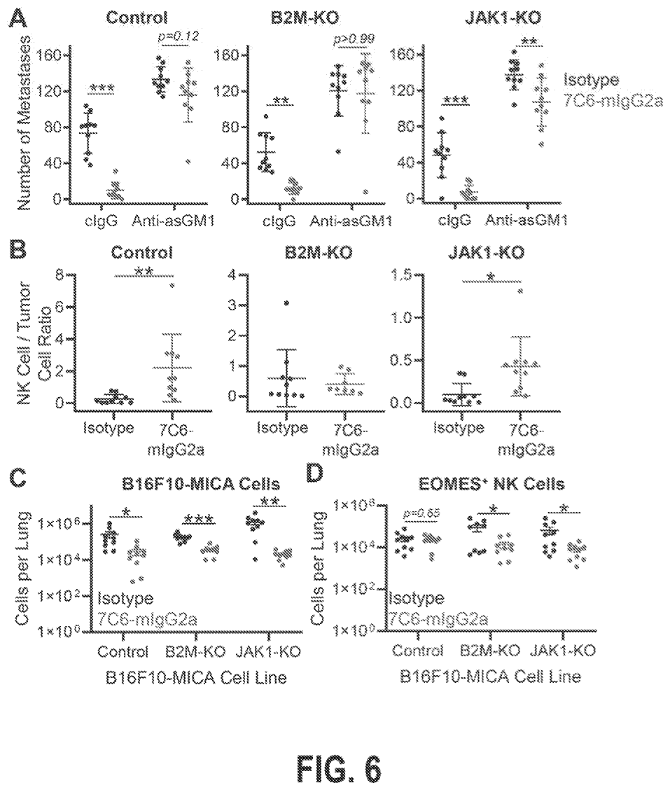

We next performed mechanistic experiments with the B16F10-MICA cell lines inoculated into WT mice. Depletion of NK cells resulted in a complete loss of MICA mAb efficacy against both control and B2m-KO B16F10-MICA tumor cells, whereas NK cell depletion greatly reduced MICA mAb efficacy against Jak1-KO B16F10-MICA tumor cells (FIG. 6). We distinguished lung-infiltrating and blood NK cells by intravenous injection of an APC-conjugated anti-CD45.2 antibody prior to euthanasia, as reported previously.²⁰ MICA mAb treatment increased the degree of NK cell infiltration into control or Jak1-KO B16F10-MICA tumors (FIG. 6). In this analysis, NK cell infiltration was normalized to tumor burden because the number of B16F10-MICA tumor cells was substantially reduced in MICA mAb treated mice (FIG. 6). These data demonstrate that MICA mAb treatment inhibits the outgrowth of melanoma metastases in a NK cell-dependent manner even when tumor cells carry inactivating mutations in B2m or Jak1 genes.

Enhanced MICA/B Surface Protein Levels on Human Tumor Cells Treated with the Combination of MICA/B mAb and HDAC Inhibitor

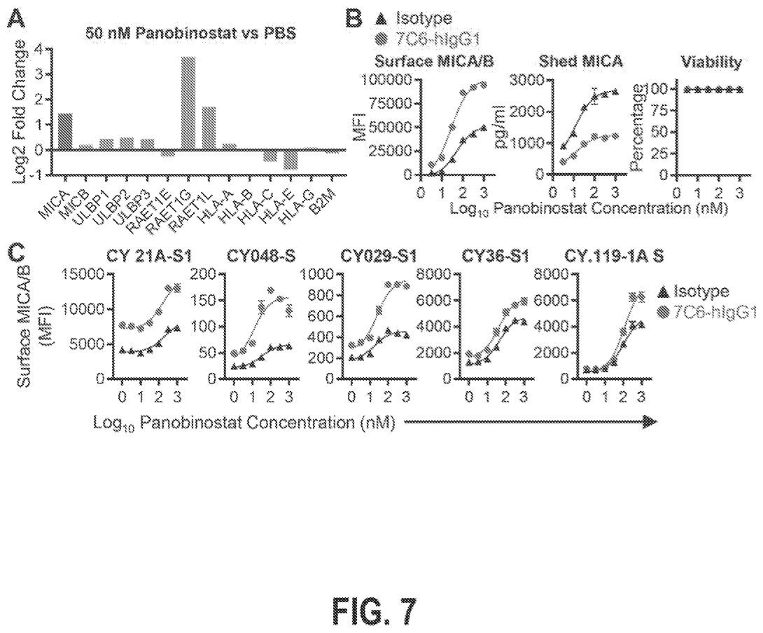

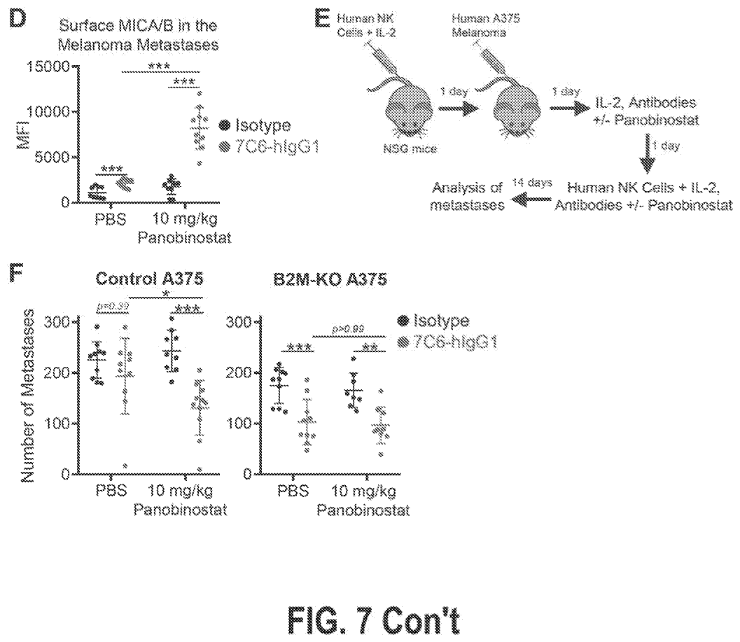

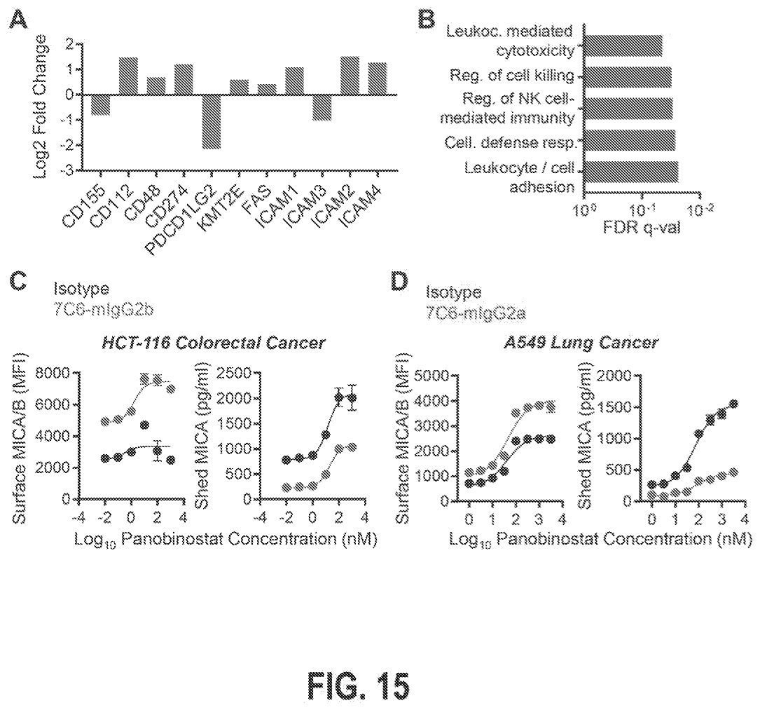

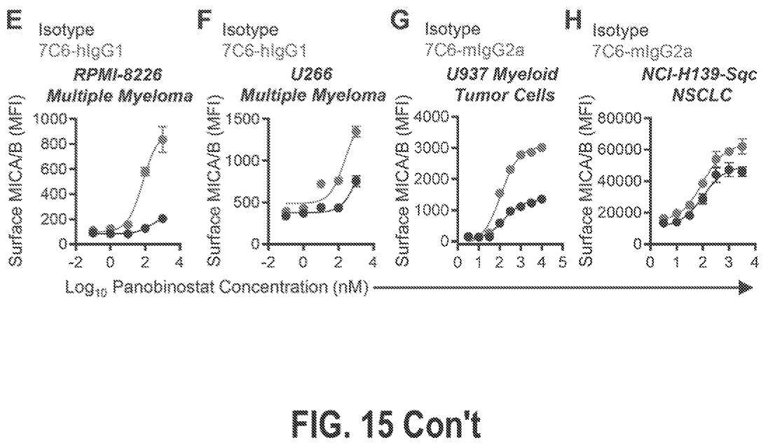

In the tumor models described above, MICA transcription was controlled by a heterologous promoter that induced relatively high levels of MICA, as previously shown.²⁰ However, in human cancers MICA/B expression is induced in response to DNA damage and cellular stress.¹³ In the human melanoma metastases that we investigated, MICA/B proteins were detectable on the surface of tumor cells in the majority of cases, albeit at a low level, and shed MICA was present in sera from 7 of 9 patients (FIG. 12). It is well known that the transcription of MICA and MICB genes is epigenetically regulated by histone deacetylases (HDACs), and that HDAC inhibitors enhance transcription of these genes.⁴² The pan-HDAC inhibitor panobinostat was approved by the U.S. Food and Drug Administration (FDA) for the treatment of multiple myeloma.⁴³ Previous work in multiple mouse models of cancer established that an intact immune system is required for the therapeutic activity of panobinostat.⁴⁴ We therefore examined whether the combination of panobinostat and 7C6-hIgG1 mAb can enhance MICA/B protein levels by increased transcription (panobinostat) and surface protein stabilization (MICA/B mAb). RNA-seq analysis demonstrated that treatment of A375 melanoma cells with panobinostat (50 nM) for 24 hours increased mRNA levels of multiple genes encoding NKG2D ligands, including MICA, RAET1G, and RAET1L. However, panobinostat did not increase mRNA levels of genes encoding classical or non-classical MHC-I molecules (FIG. 7). Panobinostat also affected transcription of many other genes in A375 melanoma cells, some of which represented immunity-related pathways (FIG. 15). Surface MICA/B protein levels were substantially increased by the combination of panobinostat and MICA/B mAb, and the concentration of shed MICA was reduced without a reduction in cellular viability (FIG. 7). These conclusions were further supported by analysis of a diverse panel of human tumor cell lines (FIG. 15). We also examined MICA/B protein levels by a panel of short-term human melanoma cell lines established from metastatic lesions.^{20, 45} Treatment with panobinostat plus MICA/B mAb substantially increased the surface density of MICA/B proteins compared to treatment with individual compounds (FIG. 7). These data demonstrate that combinatorial approaches that increase transcription of MICA/B genes and stabilize synthesized proteins result in a substantial increase of surface MICA/B proteins on human cancer cells.

The Combination of MICA mAb and Panobinostat Reduces Melanoma Metastases in Mice Reconstituted with Human NK Cells

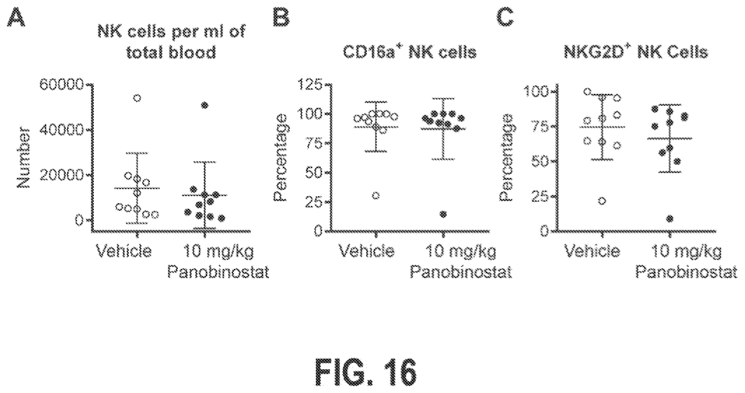

We next investigated the in vivo activity of panobinostat on surface MICA/B protein levels on human melanoma cells. We first established that the selected dose of panobinostat (10 mg/kg) did not negatively impact human NK cells transferred to immunodeficient NSG mice (based on number of circulating total NK cells as well as CD16a or NKG2D positive NK cells, FIG. 16). Next, we injected ZsGreen⁺ A375 melanoma cells intravenously into NSG mice and waited for two weeks until metastases were established. Mice were then treated twice at 24-hour intervals with panobinostat (or PBS), MICA/B mAb (or isotype control mAb) or the combination of panobinostat plus MICA/B mAb (or panobinostat plus isotype control mAb). One day later, MICA/B surface protein levels were quantified on ZsGreen⁺ tumor cells from dissociated lung tissue by flow cytometry. The selected dose of panobinostat did not significantly increase MICA/B protein levels on melanoma cells, but the combination of panobinostat and MICA/B mAb resulted in high MICA/B surface levels on ZsGreen⁺ A375 melanoma cells in pulmonary metastases (FIG. 7).

Based on our prior experience, survival of transferred human NK cells was limited in NSG mice and only a relatively small number of human NK cells infiltrated lung tissue. We therefore initiated treatment one day following inoculation of A375 melanoma cells (FIG. 7). The early start of treatment likely enabled NK cell recognition of tumor cells that had not yet infiltrated deeply into the lung tissue. We found that only the combination of panobinostat plus MICA/B mAb reduced the number of lung metastases formed by control (B2M wild-type) A375 melanoma cells, while monotherapy with either panobinostat or MICA/B mAb was ineffective. In contrast, monotherapy with the MICA/B mAb significantly reduced the number of lung metastases formed by B2M-KO A375 melanoma cells (FIG. 7). The combination of MICA/B mAb and panobinostat did not enhance this effect against B2M-KO metastases, potentially because NK cell reconstitution was limited in this model. These results demonstrate that MICA/B mAb treatment is more effective against MHC-I deficient human melanoma metastases in this humanized mouse model, whereas only the combination therapy is effective against melanoma metastases that express MHC-I protein.

Discussion

Primary and secondary resistance to checkpoint blockade are major issues in oncology. Many mechanisms of resistance to checkpoint blockade are related to the MHC-I and IFNγ signaling pathways in tumor cells. These include mutations of B2M or other genes in the MHC-I antigen presentation pathway, transcriptional and epigenetic silencing of neoantigen or MHC-I expression as well as inactivating mutations in the IFNγ signaling pathway.^{4, 5, 6} Although MICA/B proteins have a similar structure to MHC-I proteins, they do not assemble with α2-microglobulin.³⁸ Also, transcription of the MICA/B genes is regulated by DNA damage and cellular stress rather than by IFNγ.¹³ Therefore, inactivating mutations in the MHC-I and IFNγ pathway have no detrimental effect on MICA/B expression.

It is well known that loss of MHC-I expression removes an important inhibitory signal for NK cells, but sufficient activating signals are also required for induction of NK cell-mediated tumor immunity.¹² We show that metastases with inactivating mutations in the MHC-I (B2M mutation) or IFNγ signaling (JAK1 mutation) pathways can be treated with a MICA/B α3 domain specific antibody. This antibody inhibits proteolytic shedding of MICA/B, a common evasion mechanism from NKG2D receptor mediated immunity in human cancers. We previously showed that treatment with this mAb induces activation of both NKG2D (increased density of MICA/B ligand) and CD16a (Fc region of mAb) receptors on NK cells.²⁰ Without wishing to be bound by theory, two approaches can be tested for eliciting NK cell-mediated tumor immunity with such a mAb. First, a MICA/B α3 domain specific mAb can be used to treat tumors resistant to checkpoint blockade due to inactivating mutations in the MHC-I or IFNγ signaling pathways. Second, simultaneous administration of a MICA/B mAb and a PD-1 mAb can elicit simultaneously NK cell and CD8 T cell-mediated immunity against tumor cells and thereby prevent the outgrowth of tumor clones resistant to cytotoxic T cells. Such an approach can be of particular interest for advanced human tumors with extensive heterogeneity. It is important to note that the NKG2D receptor is also expressed by human CD8 T cells, γδT cells and ILCs.^{14, 46, 47} Without wishing to be bound by theory, MICA/B mAb treatment therefore can enhance T cell-mediated tumor immunity.

Many therapeutic approaches used in oncology enhance expression of MICA/B proteins by tumor cells. For example, it is well known that HDAC inhibitors enhance transcription of MICA/B genes, such as panobinostat, a FDA approved drug.⁴³ However, proteolytic shedding of MICA/B proteins by tumor cells limits the effect of such drugs on NKG2D receptor activation. We show that the combination of panobinostat and a MICA/B α3 domain antibody greatly increased MICA/B surface protein levels and enhanced NK cell-mediated immunity in a humanized mouse model of melanoma metastases. We acknowledge that this humanized model has significant limitations, in particular the limited survival of transferred human NK cells due to lack of homeostatic cytokine signaling. A similar approach can be used to develop combination therapies with other FDA approved drugs. The HDAC inhibitor can also induce expression of NKG2D ligands in healthy tissues, but this aspect cannot be evaluated in our study because mice do not have MICA B genes. It is also known that the DNA damage response induced by radiation therapy strongly enhances MICA/B transcription.⁴⁸ A combination of local radiotherapy and systemic immunotherapy with a MICA/B mAb can be attractive to limit immune-related adverse events that have been observed with combinations involving two systemic immunotherapy agents (such as PD-1 and CTLA-4 mAbs). Also, there is already clinical evidence that radiation therapy in combination with immunotherapy (CTLA-4 blockade) can induce systemic tumor immunity against non-irradiated lesions (abscopal effect).⁴⁹

The single cell data demonstrate that NK cells are indeed present in human melanoma metastases, including patients who progressed following treatment with PD-1 or CTLA-4 mAbs. Tumor and blood NK cell populations from the same patients demonstrated striking transcriptional differences. Most NK cells in blood samples (82.9%, bNK0 cluster) expressed the classical cytotoxicity signature (including GZMB and PRF1). However, NK cells isolated from melanoma metastases were more diverse in their gene expression programs: we detected 7 NK cell clusters (plus one ILC3 cluster) in tumors while one dominant and three smaller NK cell clusters were detected in blood. Most NK cells within tumors were positive for the cytotoxicity signature but with a gradient across clusters (highest in tNK.3 and tNK.4 clusters, lower in tNK.0, tNK.1 and tNK.2 clusters). This signature, for example, is a global assessment of cytokine function, which is more informative than a single marker such as granzyme A or perform. The most striking difference between tumor-infiltrating versus blood NK cells related to expression of chemokine genes. Two recent publications showed that NK cells play an important role in the recruitment of cross-presenting DCs (cDC1) to tumors by secreting the chemokines XCL1 and XCL2 that bind to the XCR1 receptor.^{24, 26} Also, NK cells and cDC1 were found to frequently interact, indicating that NK cells play an important role in recruiting DCs critical for T cell-mediated tumor immunity.²⁴ Interestingly, we found that a much larger number of tumor-infiltrating versus blood NK cells expressed XCL1 and XCL2. Furthermore, we observed functional specialization among tumor NK cell clusters in terms of chemokine gene expression: NK cells with a lower cytotoxicity signature (clusters tNK.0, tNK.1, tNK.2, tNK.6) expressed higher levels of XCL1 and XCL2 than clusters with a higher cytotoxicity signature (tNK.3 and tNK.4). Tumor-infiltrating NK cells with a higher cytotoxicity signature in fact expressed a distinct set of chemokine genes (CCL3, CCL4, CCL4L2, CCL5). The encoded chemokines bind to the CCR5 chemokine receptor and recruit T cells as well as other immune cell populations.³² These data thus indicate significant functional specialization among tumor-infiltrating NK cell populations: NK cells with a lower cytotoxicity signature tend to express high levels of XCR1 binding chemokines that recruit cDC1, while NK cells with a higher cytotoxicity signature express higher levels of CCR5 binding chemokines that recruit T cells and other immune cells. These data and recent publications demonstrate that the role of NK cells in tumor immunity needs to be reconsidered in a broader context: NK cells not only kill tumor cells but can also create favorable microenvironments by recruiting key immune cell populations required for protective tumor immunity.

A recent scRNA-seq study analyzed the transcriptome of human NK cells and identified two NK cell populations in blood and four populations in spleen samples. NK cell populations in blood versus spleen were characterized by distinct transcriptional features.⁵⁰ Without wishing to be bound by theory, this study and our data validate that the transcriptional state of NK be dynamically regulated by the tissue microenvironment. Without wishing to be bound by theory, the different NK cell populations identified in tumors localize to distinct microenvironments. The apparent functional specialization of NK cell populations within tumors also provides opportunities to enhance distinct aspects of NK cell function, such as enhancing cytotoxic activity or secretion of chemokines that recruit cross-presenting DCs.

In summary, we show that metastases with mutations that cause resistance to cytotoxic T cells can be targeted by NK cells when MICA/B shedding is inhibited with a mAb. A number of combination strategies can be tested to further enhance the activity of NK cells against metastatic lesions: 1. Approaches that enhance MICA/B protein expression by tumor cells (such as panobinostat, local radiation therapy)⁴⁸, 2. Cytokines that enhance NK cell function within tumors and reduce TGFβ-mediated NKG2D downregulation (such as IL-15/IL-15Rα complex)⁵¹, 3. Antibodies that target inhibitory receptors on NK cells⁵². The single cell data on metastasis-infiltrating human NK cells also provide a wealth of information on the gene expression programs of distinct NK cell populations. These single cell data can inform the future development of strategies for enhancing NK cell immunity against tumors resistant to cytotoxic T cells.

Methods

Cell Lines

B16F10, LLC1, A375, HCT-116, A549, and U937 cell lines were purchased from ATCC (Manassas, Va.). RPMI-8226 and U266 cell lines were generously donated (Dana-Farber Cancer Institute, Boston, Mass.), and the NCI-H139-Sqc cell line was generously donated. The CY029-S1, CY048-S, CY 21A-S1, CY.119-1A S, and CY36-S1 short-term melanoma cell lines were previously described.^{20, 45} All cell lines tested negative for mycoplasma using the Universal Mycoplasma Detection Kit (ATCC, catalog number 30-1012K) or MycoAlert™ Mycoplasma Detection Kit (Lonza, catalog number LT07-318). All cell lines were used within a small number of passages after they had been obtained from vendors or collaborators. A375, HCT-116, A549, U937, RPMI-8226, U266, and NCI-H139-Sqc cell lines were cultured in RPMI-1640 media, whereas the B16F10, LLC1, CY029-S1, CY048-S, CY 21A-S1, CY.119-1A S, and CY36-S1 in DMEM media. RPMI-1640 and DMEM media were supplemented with 10% FBS, 1× Glutamax, and 1× Penicillin/Streptomycin. All tissue culture reagents were purchased from Gibco (Thermo Fisher Scientific). Cells were cultured at 37° C. with 5% CO₂.

Control and B2M-KO A375 cells were generated by transducing parental A375 cells with a lentiCas9-blast vector followed by selection with blasticidin. Subsequently, cells were transduced with pLKO3G-gRNA-PGK-EGFP vector with a gRNA targeting the human B2M genes inserted between the BsmB1 sites; the control cell line was transduced with the backbone of the vector. Following transduction, cells were cultured for 24 hours in the presence of recombinant human IFNγ (10 ng/ml) to induce upregulation of MHC-I proteins and stained with APC-conjugated W6/32 antibody (Biolegend, catalog number 311410). HLA-A/B/C negative B2M-KO cells and HLA-A/B/C positive control cells were sorted by flow cytometry. Parental A375 cells were also transduced with the pHAGE lentiviral vector to enable expression of ZsGreen under the control of the EF1α promoter. ZsGreen⁺ A375 cells were sorted by flow cytometry and used to examine MICA/B expression in vivo.

The generation of B16F10 control and B2m-KO cell lines was previously reported.⁵³ MICA expression was achieved by transduction of control and B2m-KO B16F10 cells with the pHAGE lentiviral vector that carried a MICA*009-IRES-luciferase expression cassette under the control of the EF1α promoter, as described previously.²⁰ Cells were treated for 24 hours with IFNγ (10 ng/ml) and then labeled with APC-conjugated H-2D^{b} antibody (Biolegend, catalog number 111513) and PE-conjugated MICA 6D4 antibody (Biolegend, catalog number 320906). MICA⁺ H-2D^{b-} B2M-KO and MICA⁺ H-2D^{b+} control B16F10 were then sorted by flow cytometry.

Jak1-KO B16F10-MICA cells were generated by electroporation of the control B16F10-MICA cell line with Cas9 protein and a gRNA targeting the Jak1 gene. Electroporation was performed using the Amaxa™ SF Cell Line 96-well Nucleofector™ Kit (Lonza, V4SC-2096) in a 4D Nucleofactor (Lonza). Cells were then cultured for 24 hours with recombinant murine IFNγ (10 ng/ml), and MICA⁺ H-2D^{b-} cells were isolated by flow cytometry.

LLC1 cells were first transduced with a pHAGE lentiviral vector that carried a MICA*009 cDNA—IRES—ZsGreen expression cassette under the control of an EF1α promoter. The resulting LLC1-MICA cells were electroporated with Cas9 protein and bound gRNAs targeting the B2m gene; control cells were electroporated with Cas9 protein alone. Control and B2m-KO LLC1-MICA cells were treated with IFNγ for 24 hours and sorted by flow cytometry based on expression of H-2K^{b}. Control LLC1-MICA cells were H-2K^{b+}, whereas B2m-KO LLC1 cells were H-2K^{b-}.

MICA/B Shedding Assays

5×10⁴ tumor cells were cultured for 24 hours in 96-wells plates (flat-bottom for adherent cells or U-bottom for suspension cells) in the presence of different concentrations of antibodies, IFNγ, and/or panobinostat (ApexBio, catalog number A8178), as indicated in each figure. Following a 24-hour culture period, plates were centrifuged for 5 minutes at 500× g, and supernatants were collected for analysis of shed MICA using the Human MICA ELISA Kit (Abcam, catalog number ab59569). We previously demonstrated that the 7C6 mAb did not interfere with detection of shed MICA using this ELISA kit.²⁰

Adherent cells were detached with Versene (Gibco, catalog number 15040-066) to preserve the integrity of MICA/B proteins on the cell surface. Fc receptors were blocked using Human TruStain FcX™ (Biolegend, catalog number 422302), and cells were stained with PE or APC-conjugated anti-human MICA/B clone 6D4 (Biolegend, catalog numbers 320906 or 320908, respectively). Importantly, the 6D4 antibody binds to the α1-α2 domains of MICA/B and thereby does not compete with the 7C6 antibody that targets the 3 domain of MICA/B, as shown previously.²⁰ Cells were also stained with dead cell markers, either 7-AAD (BD Pharmingen™, catalog number 559925), Zombie UV, Yellow or Near Infrared (Biolegend, catalog numbers 423108, 423104, and 423106, respectively). Data were acquired using a BD Fortessa X20 or Beckman Coulter CytoFLEX LX, and analyses were performed using FlowJo V10 software.

Isolation of Human and Murine NK Cells

Human NK cells from healthy individuals (leukoreduction collars) were isolated by negative selection using the EasySep™ Human NK Cell Isolation Kit (Stem Cell Technologies, catalog number 17955), which resulted in NK cell purities of at least 90%. Leukoreduction collars were provided in an anonymous manner by Brigham and Women's Hospital (Boston, USA). NK cells were expanded in vitro in G-Rex 6-wells plates (Wilson Wolf, catalog number 80240M) using RPMI-1640 media supplemented with 10% FBS, 5% human AB serum, 1,000 U/ml IL-2, and 20 ng/ml IL-15; media was replenished once per week until NK cells were used for experiments.

Murine NK cells were isolated by meshing spleen tissue using a 70 μm cell strainer, followed by red cell lysis (ACK buffer) and staining with PE-conjugated anti-mouse CD49b mAb (Biolegend, catalog number 108908) and APC-conjugated anti-mouse CD3ϵ mAb (Biolegend, catalog number 100312). NK cells were sorted by flow cytometry, yielding typical purities of ˜99%. These cells were immediately injected in Rag2^{-/-} Il2rg^{-/-} KO mice for experiments involving allogeneic or syngeneic NK cells.

NK Cell-Mediated Killing Assays

For long-term NK cell-mediated killing assays, GFP⁺ control and B2M-KO A375 cells were pretreated for 24 hours with MICA/B or isotype control mAbs (20 μg/ml) in tissue culture media. Subsequently, tumor cells were detached with Versene, washed with PBS, and plated in black-wall 96-well plates (Corning™, catalog number 3603) at a density of 5×10³ cells per well. Human NK cells were added 1-2 hours later at different effector to target ratios as indicated in the figures, and IL-2 (300 U/ml) was added to support NK cell survival. The number of GFP⁺ tumor cells was tracked over time using a Celigo Image Cytometer (Nexcelom Bioscience, Lawrence, USA), as reported previously.⁴⁰

For short-term NK cell killing assays, A375 melanoma cells were pretreated for 24 hours with MICA/B or isotype control mAbs (20 μg/ml) in tissue culture media and then used as target cells in 4-hours ⁵¹Cr-release assays, as described previously.²⁰ NK cells were isolated by negative selection from leukapheresis reduction collars and cultured for 24 hours with 1,000 U/ml IL-2 in 96-wells U-bottom plates before use in the assay. In some experiments, KIR receptors on NK cells were blocked in the ⁵¹Cr-release assay by addition of isotype control mAb or anti-KIR2DL2/3 plus anti-KIR2DL4 mAbs (BioLegend, catalog numbers 312602 and 347003).

Bulk RNA-Seq Analysis of Human A375 Melanoma Cells

Parental A375 cells were cultured for 24 hours with 50 nM panobinostat or the corresponding volume of PBS. Cells were then detached with Versene, and RNA was isolated with RNeasy Plus Mini Kit and RNase-Free DNase Set, respectively (both Qiagen kits, catalog numbers 74134 and 79254, respectively). Generation of cDNA, sequencing, and analyses were done as previously reported.²⁰

Mice

Wild Type (WT) C57BL6/J, Ighm^{-/-} C57BL6/J, CB6F1/J and NSG mice were purchased from the Jackson Laboratories (catalog numbers 000664, 002288, 100007, and 005557, respectively). Rag2^{-/-} Il2rg^{-/-} knockout mice were purchased from Taconic (catalog number 4111). Mice were male (except for NSG mice that were female) and 6-8 weeks of age. All mice were housed in the vivarium of the Dana-Farber Cancer Institute as previously reported.²⁰ The institutional committee for animal use approved the procedures used in this study (animal protocol number 08-049).

Metastasis Models in Immunocompetent Mice

B16F10-MICA tumor cells (control, B2m-KO or Jak1-KO) were inoculated intravenously into C57BL/6 mice (WT or Ighm^{-/-}) via the tail vein (1 to 7×10⁵ cells in 100 μl of PBS depending on the experiment, as described in figure legends). Treatment was initiated in Ighm^{-/-} mice when mice had established metastases (day 7 following tumor inoculation) by intraperitoneal injection of isotype control mAb (BioXcell, catalog number BE0085) or 7C6-mIgG2a mAb (200 μg per injection, days 7, 8 and then once per week). In an alternative protocol, WT mice received antibody injections on days 1, 2 and then once per week. Antibodies that induced depletion of CD8 T cells (100 μg anti-CD80, BioXcell, catalog BE0223) or NK cells (1:10 dilution anti-asialo GM1, Wako Chemicals, catalog 986-10001) were injected on days −1, 0 and then once per week relative to tumor cell inoculation; murine IgG1 was used as control IgG (BioXcell, catalog BE0083). Lung metastases were quantified on day 14 under a stereomicroscope following formalin fixation of the tissue. Alternatively, the survival of mice was recorded.

For the LLC1-MICA metastasis model, WT C57BL/6 mice were inoculated intravenously via the tail vein with 1.0 to 1.5×10⁶ tumor cells (as indicated in the figure legends) in 0.1 ml of PBS. 7C6-mIgG2a or isotype control mAbs (200 μg) were administered on days 2, 3 and then once per week relative to tumor cell inoculation. For experiments involving adoptive transfer of NK cells, 2×10⁵ NK cells isolated from WT C57BL/6 (syngeneic) or CB6F1/J (allogeneic) mice were injected intravenously into Rag2^{-/-} Il2rg^{-/-} knockout mice. These NK cells were isolated by flow cytometry as CD3ϵ⁻ CD49b⁺ cells. LLC1-MICA tumor cells (7×10⁵ cells) were injected intravenously one day following NK cell transfer. 7C6-mIgG2a or isotype control mAbs (200 μg/injection) were given intraperitoneally on days 2, 3 and then once per week. On day 14, mice (WT or Rag2^{-/-} Il2rg^{-/-} knockout) were euthanized by CO₂ inhalation, and Indian ink (30%) was injected into the trachea to enable counting of lung metastases, as previously described.²⁸ Lung tissue was treated using Fekete's fixative and surface metastases were counted using a stereomicroscope.

Characterization of Murine NK Cells in the B16F10-MICA Metastasis Model

WT C57BL/6 mice were inoculated intravenously with 7×10⁵ B16F10-MICA cells with a control, B2m-KO or Jak1-KO genotype. Mice were treated with 7C6-mIgG2a or isotype control mAbs (200 μg/injection) on days 1 and 3 following tumor cell inoculation. On day 12, mice were injected intravenously with 50 μl of APC-conjugated anti-mouse CD45.2 (Biolegend, 109814) to label intravascular immune cells and then euthanized. Lung tissue was cut into small pieces, resuspended in RPMI-1640 supplemented with 1 mg/ml collagenase type IV, 0.1 mg/ml hyaluronidase and 20 U/ml DNase, and processed using a gentle MACS instrument (Miltenyi). The cell suspension was then incubated with mouse TruStain FcX™ (Biolegend, catalog number 101320) and multiple antibodies, including PE-Cy7-conjugated anti-mouse CD45.2 (Biolegend, 109830), APC-conjugated anti-mouse CD3ϵ (Biolegend, 100312), APC-conjugated anti-mouse TCRβ (Biolegend, 109212), BV785-conjugated anti-mouse NK1.1 (BD Biosciences, 740853), PE-CF594-conjugated anti-mouse CD49b (BD Biosciences, 562453), Alexa488-conjugated anti-mouse EOMES (Invitrogen, 53-4875-82), PE-conjugated anti-mouse GZMA (Invitrogen, 12-5831-82), BV421-conjugated anti-mouse NKG2D (BD Biosciences, 562800), PERCP-CY5.5-conjugated anti-mouse CD16/32 (Biolegend, 101324), BV510-conjugated anti-mouse Ly49C/I (BD Biosciences, 744028) and Zombie UV. Cells were analyzed using a CytoFLEX Flow Cytometer (Beckman Coulter), and data were processed using FlowJo V10.

Humanized Mouse Model

NSG mice were reconstituted intravenously with human NK cells (1 to 2×10⁶ cells) that had been expanded in vitro as described above; IL-2 was injected intraperitoneally (7.5×10⁴ units) to support in vivo survival of NK cells, as previously reported.²⁰ A375 melanoma cells (5×10⁵ cells, control or B2M-KO) were injected one day later (day 0). One day after tumor cell inoculation, mice received another dose of IL-2, plus isotype control (BioXcell, catalog BE0096) or 7C6-hIgG1 mAbs (200 μg) as well as PBS or 10 mg/kg panobinostat (ApexBio, catalog number A8178). On day 2, mice were again reconstituted with human NK cells from the same donor and also received injections of IL-2, antibodies as well as PBS or panobinostat. Metastases were quantified 2 weeks after the last treatment as described above for the LLC1-MICA metastasis model (injection of Indian ink into the trachea, treatment of lung tissue with Fekete's fixative).

NSG mice were inoculated with 1×10⁶ ZsGreen⁺ A375 melanoma cells to study the effect of MICA/B mAb and panobinostat treatment on MICA/B surface levels in lung metastases; these mice did not receive human NK cells. When metastases were established (two weeks later), mice were treated on two subsequent days with 7C6-hIgG1 or isotype control mAbs (200 μg) as well as panobinostat (10 mg/kg) or PBS as a solvent control. One day following the last treatment, mice were euthanized by CO₂ inhalation, and lung tissue was dissociated mechanically to preserve the integrity of MICA/B proteins. Tumor cells were identified as viable large cells that were ZsGreen positive but negative for the murine CD45 antigen. MICA/B surface protein was labeled with 6D4-PE mAb (Biolegend, catalog 320906) and quantified by flow cytometry.

Characterization of Human NK Cells in Tumor-Free NSG Mice

Tumor-free NSG mice were inoculated intravenously with 2×10⁶ human NK cells that were expanded in vitro as described above. At the same time, mice also received intraperitoneal injections of IL-2 (7.5×10⁴) as well as panobinostat (10 mg/kg) or PBS as the solvent control. One day later, blood was collected via eye bleeding, and human NK cells were analyzed by flow cytometry.

Characterization of Human NK Cells Infiltrating Melanoma Metastases

Melanoma tissue samples were obtained from patients who required surgery for treatment of non-responsive lesions at Brigham & Women's Hospital. Blood samples were also collected at the time of surgery. Freshly resected tumor tissue was dissociated using a Tumor Dissociation Kit (Miltenyi Biotec, catalog 130-095-929). Red blood cells in blood samples were lysed using ACK buffer. NK cells were isolated using a BD Aria flow cytometer (BD Biosciences) from melanoma lesions and corresponding blood samples as lymphocyte-size single viable cells that were CD45⁺, CD56⁺, CD3ϵ⁻, CD4⁻, CD8a⁻, CD14⁻, CD15⁻, and CD163⁻. These sorted NK cells were used for scRNA-seq analysis.

In follow-up experiments, tumor and blood NK cells were also stained with anti-FGFPB2, GZMA, GZMK, CD62L, NKG2D, and CD16a antibodies and analyzed by flow cytometry. All fluorochrome-labelled antibodies were purchased from BioLegend, BD Biosciences, or eBiosciences. For intracellular staining, NK cells were fixed and permeabilized with True-Nuclear™ Transcription Factor Buffer Set (Biolegend, catalog number 424401) according to the recommendations of the manufacturer. Samples were analyzed using a Cytoflex flow cytometer (Beckman Coulter).

Single-Cell RNA-Seq

Immediately after sorting of NK cells (approximately 13,000 NK cells per sample), cell suspensions were washed in 0.05% RNase-free BSA in PBS. The 10× Genomics 3′ V2 single cell assay (10× Genomics) was used for construction of scRNA-seq libraries. Reverse transcription, cDNA amplification and library preparation were all performed according to the manufacturer's instructions. Libraries were sequenced using an Illumina HiSeq 2500 on rapid-run mode, which yielded >25,000 reads per cell.

Computational Analysis of Single-Cell RNA-Seq Data

We used 10× Genomics' Cell Ranger software for the demultiplexing, alignment, filtering, barcode counting and unique molecular identifiers (UMI) counting steps. The analysis was performed using the Seurat 3.0 package.⁵⁴ We first processed each individual data set separately prior to combining data from multiple samples. For each data set, we selected the 1,500 most variable genes. Subsequently, we ran principal component analysis (PCA) and used the first 15 principle components (PCs) to perform Louvain clustering and Uniform Manifold Approximation and Projection (UMAP) embedding.^{55, 56} We checked the most significant marker genes for each cluster to identify potential contaminating cell populations such as T cells (CD3D, CD3E and CD3G), B cells (IGHG1, IGHG2 and JCHAIN), macrophages (LYZ) and melanoma cells (MLANA); these cells were removed prior to subsequent analyses.

We then compared the paired blood and melanoma-infiltrating NK cell populations of each patient using Seurat's integration algorithm, and then also separately integrated the three melanoma-infiltrating NK cell samples and three blood NK cell samples. We used the 3,000 most variable genes from each sample and the first 15 PCs to choose 1,000 anchor genes for integration. Afterwards, we repeated PCA, clustering (resolution=0.3) and UMAP embedding on the integrated data sets. Finally, we performed differential tests on the integrated data sets to identify the genes significantly up-regulated in each cluster compared to all other cells (adjusted P<0.05), as well as the genes differentially expressed between blood and melanoma-infiltrating NK cells within each major cluster.

For gene sets representing specific cellular functions or pathways, we also computed AUCell scores for each cell in order to evaluate the variation of gene sets' activity across cell population.⁵⁷ We handpicked gene sets for cytotoxicity and chemokine activity based on analysis of the data. For NK cell, ILC1, ICL2, and ILC3 identities, we referred to the gene signatures defined by a previous study using the genes significantly up-regulated in one of these innate cell types compared to the other three populations (adjusted P<1e-3); specifically for ILC identities, the same genes were not significantly up-regulated in NK cells (adjusted P>1e-2).³¹

Statistical Analyses

All statistical analyses were performed using GraphPad Prism 8 software, and the relevant statistical tests are indicated in each figure legend.

### REFERENCES CITED IN THIS EXAMPLE

1. Ribas, A. & Wolchok, J. D. Cancer immunotherapy using checkpoint blockade. Science 359, 1350-1355 (2018).
2. Schumacher, T. N. & Schreiber, R. D. Neoantigens in cancer immunotherapy. Science 348, 69-74 (2015).
3. Parker, B. S., Rautela, J. & Hertzog, P. J. Antitumour actions of interferons: implications for cancer therapy. Nature Reviews Cancer 16, 131-144 (2016).
4. Gao, J. et al. Loss of IFN-γ pathway genes in tumor cells as a mechanism of resistance to anti-CTLA-4 therapy. Cell 167, 397-404. e399 (2016).
5. Zaretsky, J. M. et al. Mutations associated with acquired resistance to PD-1 blockade in melanoma. New England Journal of Medicine 375, 819-829 (2016).
6. Pitt, J. M. et al. Resistance mechanisms to immune-checkpoint blockade in cancer: tumor-intrinsic and -extrinsic factors. Immunity 44, 1255-1269 (2016).
7. Snyder, A. et al. Genetic basis for clinical response to CTLA-4 blockade in melanoma. New England Journal of Medicine 371, 2189-2199 (2014).
8. Rizvi, N. A. et al. Mutational landscape determines sensitivity to PD-1 blockade in non-small cell lung cancer. Science 348, 124-128 (2015).
9. Hellmann, M. D. et al. Nivolumab plus ipilimumab in lung cancer with a high tumor mutational burden. New England Journal of Medicine (2018).
10. Hellmann, M. D. et al. Tumor mutational burden and efficacy of nivolumab monotherapy and in combination with ipilimumab in small-cell lung cancer. Cancer cell 33, 853-861. e854 (2018).
11. June, C. H., O'Connor, R. S., Kawalekar, O. U., Ghassemi, S. & Milone, M. C. CAR T cell immunotherapy for human cancer. Science 359, 1361-1365 (2018).
12. Long, E. O., Sik Kim, H., Liu, D., Peterson, M. E. & Rajagopalan, S. Controlling natural killer cell responses: integration of signals for activation and inhibition. Annual review of immunology 31, 227-258 (2013).
13. Raulet, D. H., Gasser, S., Gowen, B. G., Deng, W. & Jung, H. Regulation of ligands for the NKG2D activating receptor. Annu Rev Immunol 31, 413-441 (2013).
14. Bauer, S. et al. Activation of NK cells and T cells by NKG2D, a receptor for stress-inducible MICA. Science 285, 727-729 (1999).
15. Salih, H. R., Rammensee, H.-G. & Steinle, A. Cutting edge: down-regulation of MICA on human tumors by proteolytic shedding. The Journal of Immunology 169, 4098-4102 (2002).
16. Kaiser, B. K. et al. Disulphide-isomerase-enabled shedding of tumour-associated NKG2D ligands. Nature 447, 482-486 (2007).
17. Jinushi, M. et al. MHC class I chain-related protein A antibodies and shedding are associated with the progression of multiple myeloma. Proceedings of the National Academy of Sciences 105, 1285-1290 (2008).
18. Yang, F. et al. Matrix metallopeptidase 2 (MMP2) mediates MHC class I polypeptide-related sequence A (MICA) shedding in renal cell carcinoma. Actas Urológicas Españolas (English Edition) 38, 172-178 (2014).
19. Boutet, P. et al. Cutting edge: the metalloproteinase ADAM17/TNF-α-converting enzyme regulates proteolytic shedding of the MHC class I-related chain B protein. The Journal of Immunology 182, 49-53 (2009).
20. de Andrade, L. F. et al. Antibody-mediated inhibition of MICA and MICB shedding promotes NK cell-driven tumor immunity. Science 359, 1537-1542 (2018).
21. Trefny, M. P. et al. A Variant of a Killer Cell Immunoglobulin-like Receptor is Associated with Resistance to PD-1 Blockade in Lung Cancer. Clinical Cancer Research, clincanres. 3041.2018 (2019).
22. Hsu, J. et al. Contribution of NK cells to immunotherapy mediated by PD-1/PD-L1 blockade. J Clin Invest (2018).
23. Rodig, S. J. et al. MHC proteins confer differential sensitivity to CTLA-4 and PD-1 blockade in untreated metastatic melanoma. Science translational medicine 10, eaar3342 (2018).
24. Barry, K. C. et al. A natural killer-dendritic cell axis defines checkpoint therapy-responsive tumor microenvironments. Nature Medicine, 1 (2018).
25. Cursons, J. et al. A natural killer cell gene signature predicts melanoma patient survival. bioRxiv, 375253 (2018).
26. Bottcher, J. P. et al. NK cells stimulate recruitment of cDC1 into the tumor microenvironment promoting Cancer immune control. Cell 172, 1022-1037. e1014 (2018).
27. Souza-Fonseca-Guimaraes, F., Cursons, J. & Huntington, N. D. The Emergence of Natural Killer Cells as a Major Target in Cancer Immunotherapy. Trends in immunology (2019).
28. Ferrari de Andrade, L. et al. Natural killer cells are essential for the ability of BRAF inhibitors to control BRAFV600E-mutant metastatic melanoma. Cancer research 74, 7298-7308 (2014).
29. Chen, L. et al. CD56 Expression Marks Human Group 2 Innate Lymphoid Cell Divergence from a Shared NK Cell and Group 3 Innate Lymphoid Cell Developmental Pathway. Immunity 49, 464-476. e464 (2018).
30. Gasteiger, G., Fan, X., Dikiy, S., Lee, S. Y. & Rudensky, A. Y. Tissue residency of innate lymphoid cells in lymphoid and nonlymphoid organs. Science 350, 981-985 (2015).
31. Björklund, Å. K. et al. The heterogeneity of human CD127+ innate lymphoid cells revealed by single-cell RNA sequencing. Nature immunology 17, 451 (2016).
32. Chow, M. T. & Luster, A. D. Chemokines in cancer. Cancer immunology research 2, 1125-1131 (2014).
33. Akatsuka, A. et al. Tumor cells of non-hematopoietic and hematopoietic origins express activation-induced C-type lectin, the ligand for killer cell lectin-like receptor F1. International immunology 22, 783-790 (2010).
34. da Silva, I. P. et al. Reversal of NK-cell exhaustion in advanced melanoma by Tim-3 blockade. Cancer immunology research 2, 410-422 (2014).
35. Fregni, G. et al. Phenotypic and functional characteristics of blood natural killer cells from melanoma patients at different clinical stages. PLoS One 8, e76928 (2013).
36. Lee, J.-C., Lee, K.-M., Kim, D.-W. & Heo, D. S. Elevated TGF-β1 secretion and down-modulation of NKG2D underlies impaired NK cytotoxicity in cancer patients. The Journal of Immunology 172, 7335-7340 (2004).
37. Marrufo, A. M. et al. Blocking LLT1 (CLEC2D, OCIL)-NKRP1A (CD161) interaction enhances natural killer cell-mediated lysis of triple-negative breast cancer cells. American journal of cancer research 8, 1050 (2018).
38. Li, P. et al. Complex structure of the activating immunoreceptor NKG2D and its MHC class I-like ligand MICA. Nature immunology 2, 443-451 (2001).
39. Bahram, S., Bresnahan, M., Geraghty, D. E. & Spies, T. A second lineage of mammalian major histocompatibility complex class I genes. Proceedings of the National Academy of Sciences 91, 6259-6263 (1994).
40. Chan, L. L.-Y., Wucherpfennig, K. & De Andrade, L. F. Visualization and quantification of NK cell-mediated cytotoxicity over extended time periods by image cytometry. Journal of Immunological Methods (2019).
41. Ruggeri, L. et al. Effectiveness of donor natural killer cell alloreactivity in mismatched hematopoietic transplants. Science 295, 2097-2100 (2002).
42. Skov, S. et al. Cancer cells become susceptible to natural killer cell killing after exposure to histone deacetylase inhibitors due to glycogen synthase kinase-3-dependent expression of MHC class I-related chain A and B. Cancer research 65, 11136-11145 (2005).
43. Laubach, J. P., Moreau, P., San-Miguel, J. F. & Richardson, P. G. Panobinostat for the treatment of multiple myeloma. Clinical cancer research, clincanres. 0530.2015 (2015).
44. West, A. C. et al. An intact immune system is required for the anticancer activities of histone deacetylase inhibitors. Cancer research (2013).
45. Izar, B. et al. Bidirectional cross talk between patient-derived melanoma and cancer-associated fibroblasts promotes invasion and proliferation. Pigment cell & melanoma research 29, 656-668 (2016).
46. Groh, V., Steinle, A., Bauer, S. & Spies, T. Recognition of stress-induced MHC molecules by intestinal epithelial γδ T cells. Science 279, 1737-1740 (1998).
47. Chiossone, L., Dumas, P.-Y., Vienne, M. & Vivier, E. Natural killer cells and other innate lymphoid cells in cancer. Nature reviews Immunology, 1 (2018).
48. Dhar, P. & Wu, J. D. NKG2D and its ligands in cancer. Current opinion in immunology 51, 55-61 (2018).
49. Formenti, S. C. et al. Radiotherapy induces responses of lung cancer to CTLA-4 blockade. Nature medicine 24, 1845 (2018).
50. Crinier, A. et al. High-dimensional single-cell analysis identifies organ-specific signatures and conserved NK cell subsets in humans and mice. Immunity 49, 971-986. e975 (2018).
51. Romee, R. et al. First-in-human phase 1 clinical study of the IL-15 superagonist complex ALT-803 to treat relapse after transplantation. Blood, blood-2017-2012-823757 (2018).
52. André, P. et al. Anti-NKG2A mAb is a checkpoint inhibitor that promotes anti-tumor immunity by unleashing both T and NK cells. Cell 175, 1731-1743. e1713 (2018).
53. Pan, D. et al. A major chromatin regulator determines resistance of tumor cells to T cell-mediated killing. Science 359, 770-775 (2018).
54. Stuart, T. et al. Comprehensive integration of single cell data. BioRxiv, 460147 (2018).
55. Waltman, L. & Van Eck, N. J. A smart local moving algorithm for large-scale modularity-based community detection. The European Physical Journal B 86, 471 (2013).
56. McInnes, L., Healy, J. & Melville, J. Umap: Uniform manifold approximation and projection for dimension reduction. arXiv preprint arXiv:1802.03426 (2018).
57. Aibar, S. et al. SCENIC: single-cell regulatory network inference and clustering. Nature methods 14, 1083 (2017).

### Example 3—Inhibition of MICA and MICB Shedding Elicits NK Cell-Mediated Immunity Against Tumors Resistant to Cytotoxic T Cells

Abstract

Resistance to cytotoxic T cells can be mediated by loss of MHC class I expression or IFNγ signaling in tumor cells, such as mutations of B2M or JAK1 genes. Without wishing to be bound by theory, NK cells can target resistant tumors, but NK cell-based strategies remain to be developed. Without wishing to bound by theory, tumors can be targeted by NK cells if activating signals are provided. Human tumors express the MICA and MICB ligands of the activating NKG2D receptor, but proteolytic shedding of MICA/B represents an immune evasion mechanism in many human cancers. We show that B2M and JAK1 deficient metastases are targeted by NK cells following treatment with a mAb that blocks MICA/B shedding. Furthermore, we demonstrate that the FDA-approved HDAC inhibitor panobinostat and a MICA/B antibody act synergistically to enhance MICA/B surface levels on tumor cells: the HDAC inhibitor enhances MICA/B gene expression while the MICA/B antibody stabilizes the synthesized protein on the cell surface. The combination of panobinostat and the MICA/B antibody reduces the number of pulmonary metastases formed by a human melanoma cell line in NSG mice reconstituted with human NK cells. NK cell-mediated immunity induced by a mAb specific for MICA/B therefore provides an opportunity to target tumors with mutations that render them resistant to cytotoxic T cells.

Introduction

Checkpoint blockade with antibodies targeting the programmed cell death protein 1 (PD-1) or cytotoxic T-lymphocyte-associated protein 4 (CTLA-4) inhibitory receptors on T cells can induce durable anti-tumor immunity even in patients with advanced cancer. However, many patients fail to benefit from these therapies due to primary or secondary resistance (1). Cytotoxic T cells play a role in the efficacy of checkpoint blockade based on their ability to recognize tumor-derived peptides bound to major histocompatibility complex class I (MHC-I) proteins (2). Recognition of such MHC-I—peptide complexes by the T cell receptor (TCR) triggers T cell-mediated killing via release of cytotoxic granules that contain perform and granzymes. Also, secretion of interferon-γ (IFNγ) by T cells inhibits tumor cell proliferation and enhances the expression of MHC-I proteins on both tumor and dendritic cells (3). Resistance to checkpoint blockade is therefore mediated by loss of MHC-I expression by tumor cells, either by mutation or epigenetic silencing of key genes in the MHC-I (B2M, TAP1, TAP2 and other genes) or IFNγ (JAK1, JAK2) pathways (4-6). A low number or loss of neoantigens also diminishes tumor immunity mediated by cytotoxic T cells (7-10). There are currently no alternative immunotherapies for patients with solid tumors resistant to checkpoint blockade. Without wishing to be bound by theory, chimeric antigen receptor (CAR) T cells can target tumor cells that lack MHC-I proteins, but thus far CAR T cells have shown limited efficacy against solid tumors (11).

Natural Killer (NK) cells recognize tumor cells by molecular mechanisms that differ from those required by cytotoxic T cells. NK cell recognition of tumor cells is mediated by germline-encoded activating receptors that bind to ligands upregulated on tumor cells by cellular processes associated with malignant transformation, including DNA damage and cellular stress (12). In contrast, T cells recognize MHC-presented peptides derived from shared tumor antigens or neoantigens created by somatic mutations (2). Therefore, tumors resistant to cytotoxic T cells can respond to NK cell-based immunotherapy approaches. In fact, loss of MHC-I expression by tumor cells renders them more sensitive to NK cells because MHC-I proteins serve as ligands for inhibitory NK cell receptors (12). However, induction of NK cell-mediated tumor immunity can also require effective targeting of immune evasion mechanisms that hinder NK cell-mediated attack of tumor cells. For example, many human cancers express the MHC class I chain-related polypeptide A (MICA) and MICB (MICA/B) proteins that serve as ligands for the activating NK group 2D (NKG2D) receptor on NK cells and subpopulations of T cells (13, 14). Tumors evade NKG2D receptor-mediated tumor immunity by proteolytic shedding of MICA/B proteins (15-22). We developed monoclonal antibodies (mAbs) that bind to the α3 domain of MICA/B, a domain essential for shedding. These mAbs inhibited MICA/B shedding and induced NK cell-mediated tumor immunity. The increased density of MICA/B proteins on tumor cells enhanced NKG2D receptor-mediated activation of NK cells, and the Fc segment of tumor-bound antibodies also activated NK cells through the CD16 Fc receptor. Treatment with such MICA/B antibodies induced a striking shift of tumor-infiltrating NK cells to a highly cytotoxic state (23).

The MICA and MICB genes are part of the MHC locus on human chromosome 6, and the encoded proteins share significant structural similarity with MHC proteins. B2M deficiency abrogates T cell-mediated immunity and responsiveness to T cell checkpoint blockade, but MICA/B proteins do not associate with β2 microglobulin or peptides (5, 24-26). Without wishing to be bound by theory, inhibition of MICA/B shedding induces NK cell mediated immunity against metastatic lesions resistant to cytotoxic T cells. Indeed, treatment with a mAb specific for the MICA/B α3 domain enabled NK cell-mediated immunity against tumors with inactivating mutations in the MHC-I or IFNγ signaling pathways (B2m and Jak1 mutations, respectively). Also, the MICA/B genes are epigenetically regulated by histone deacetylases, which inhibit MICA/B expression by tumor cells (27-30). We found that a HDAC inhibitor acted synergistically with a MICA/B mAb in vivo to enhance MICA/B protein levels on the surface of tumor cells through enhanced transcription of MICA/B genes (HDAC inhibitor) and inhibition of MICA/B shedding (MICA/B mAb). This combination therapy conferred NK cell-mediated immunity against melanoma metastases in a humanized mouse model. Therefore, NK cell-based immunotherapies that trigger activating receptors can be used to treat cancers resistant to cytotoxic T cells.

Materials and Methods

Cell Lines

B16F10, LLC1, A375, HCT-116, A549, and U937 cell lines were purchased from ATCC (Manassas, Va.). RPMI-8226 and U266 cell lines were donated (Dana-Farber Cancer Institute, Boston, Mass.), and the NCI-H139-Sqc cell line was generously donated. The CY029-S1, CY048-S, CY 21A-S1, CY.119-1A S, and CY36-S1 short-term melanoma cell lines were previously described (23, 31). All cell lines were tested negative for mycoplasma prior to use in experiments using the Universal Mycoplasma Detection Kit (ATCC, catalog number 30-1012K) or MycoAlert™ Mycoplasma Detection Kit (Lonza, catalog number LT07-318). All cell lines were used within a small number of passages (approximately less than 10 passages) after they had been obtained from vendors or collaborators. A375, HCT-116, A549, U937, RPMI-8226, U266, and NCI-H139-Sqc cell lines were cultured in RPMI-1640 media, whereas the B16F10, LLC1, CY029-S1, CY048-S, CY 21A-S1, CY.119-1A S, and CY36-S1 were grown in DMEM media. RPMI-1640 and DMEM media were supplemented with 10% FBS, 1× Glutamax, and 1× Penicillin/Streptomycin. All tissue culture reagents were purchased from Gibco (Thermo Fisher Scientific). Cells were cultured at 37° C. with 5% CO₂.

Control and B2M-KO A375 cells were generated by transducing parental A375 cells with a lentiCas9-blast vector (Addgene #52962) followed by selection with blasticidin (Gibco, catalog number R21001). Subsequently, cells were transduced with pLKO3G-gRNA-PGK-EGFP vector, which was reported previously (32), with a gRNA targeting the human B2M genes inserted between the BsmB1 sites; the control cell line was transduced with the backbone of the vector. Following transduction, cells were cultured for 24 hours in the presence of recombinant human IFNγ (10 ng/ml, BD Biosciences) to induce upregulation of MHC-I proteins. Cells were stained with APC-conjugated W6/32 antibody (Biolegend, catalog number 311410), and HLA-A/B/C negative B2M-KO cells and HLA-A/B/C positive control cells were sorted by flow cytometry. Parental A375 cells were also transduced with a pHAGE lentiviral vector to enable expression of ZsGreen under the control of the EF1α promoter. This vector was generated by inserting the ZsGreen sequence between the NotI and BamHI restriction sites which removed an IRES and ZsGreen sequence from a parental vector. ZsGreen⁺ A375 cells were sorted by flow cytometry and used to examine MICA/B expression in vivo.

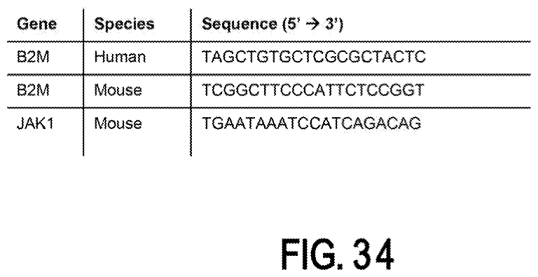

The B16F10 control and B2m-KO cell lines were previously reported (32). MICA expression was achieved by transduction of control and B2m-KO B16F10 cells with a pHAGE lentiviral vector that carried a MICA*009-IRES-luciferase expression cassette under the control of the EF1α promoter; this plasmid that was reported previously (23). The B16F10 control and B2m-KO cell lines were labelled with a PE-conjugated anti-MICA/B antibody (Biolegend catalog number 320906) and MICA⁺ cells were sorted by flow cytometry. Jak1-KO B16F10-MICA cells were generated by electroporation of the control B16F10-MICA cell line with Cas9 protein and a gRNA targeting the Jak1 gene (FIG. 34). Electroporation was performed using the Amaxa™ SF Cell Line 96-well Nucleofector™ Kit (Lonza, V4SC-2096) in a 4D Nucleofactor (Lonza). Cells were treated for 24 hours with IFNγ (10 ng/ml, BD Biosciences). Subsequently, cells were labeled with PE-conjugated MICA 6D4 antibody (Biolegend, catalog number 320906) and a cocktail of APC-conjugated anti-MHC-I antibodies (anti-H-2K^{b} and anti-H-2D^{b}, Biolegend catalog numbers 116518 and 111513, respectively). MICA⁺ MHC-I⁻ B2M-KO and MICA⁺ MHC-I⁺ control B16F10 were then sorted by flow cytometry.

LLC1 cells were first transduced with a pHAGE lentiviral vector that carried a MICA*009 cDNA—IRES—ZsGreen expression cassette under the control of an EF1α promoter (Addgene #114007). The resulting LLC1-MICA cells were electroporated with Cas9 protein and bound gRNAs targeting the B2m gene; control cells were electroporated with Cas9 protein alone. Control and B2m-KO LLC1-MICA cells were treated with IFNγ (BD Biosciences) for 24 hours and sorted by flow cytometry based on expression of H-2K^{b} (clone AF6-88.5 Biolegend). Control LLC1-MICA cells were H-2K^{b} positive whereas B2m-KO LLC1 cells were H-2K^{b} negative.

Western Blotting

B16F10, LLC1, and A375 cell lines were treated with or without 50 ng/ml IFNγ (BD Biosciences) for 16 hours. Subsequently, cells were washed in PBS and lysed in RIPA buffer (Thermo Scientific) supplemented with a protease inhibitor cocktail (Sigma Aldrich). Lysates were centrifuged for 10 minutes at 14,000 rpm, 4° C. Total protein was measured by a bicinchoninic acid assay (Thermo Scientific) and normalized prior to gel loading. Following SDS-PAGE, samples were transferred to polyvinylidene difluoride membrane, which was blocked in 5% milk in Tris-buffered saline supplemented with 0.10% Tween and then incubated overnight with the appropriate primary antibodies, as follows: anti-mouse B2M (R&D Systems), anti-human B2M, JAK1, GAPDH, and tubulin (all from Cell Signaling Technology). Following incubation with secondary antibodies conjugated with horseradish peroxidase (Cell Signaling Technology and Jackson Immunoresearch), proteins were visualized by chemiluminescence (Western Lightning and Perkin-Elmer) using a Chemi-Doc instrument (Bio-Rad Laboratories).

MICA/B Shedding Assays

5×10⁴ tumor cells were cultured for 24 hours in 96-wells plates (flat-bottom for adherent cells or U-bottom for suspension cells) in the presence of different concentrations of antibodies, IFNγ, and/or panobinostat (ApexBio, catalog number A8178), as indicated in each figure. Following a 24-hour culture period, plates were centrifuged for 5 minutes at 500×g, and supernatants were collected for analysis of shed MICA using the Human MICA ELISA Kit (Abcam, catalog number ab59569). Importantly, we previously demonstrated that the 7C6 mAb did not interfere with detection of shed MICA using this ELISA (23).

Adherent cells were detached with Versene (Gibco, catalog number 15040-066) to preserve the integrity of MICA/B proteins on the cell surface. Fc receptors were blocked using Human TruStain FcX™ (Biolegend, catalog number 422302), and cells were stained with PE or APC-conjugated anti-human MICA/B clone 6D4 (Biolegend, catalog numbers 320906 or 320908, respectively). The 6D4 antibody binds to the α1-α2 domains of MICA/B and thereby does not compete with the 7C6 antibody that targets the α3 domain of MICA/B, as shown previously (23). Cells were also stained with dead cell markers, either 7-AAD (BD Pharmingen™, catalog number 559925), Zombie UV, Yellow or Near Infrared (Biolegend, catalog numbers 423108, 423104, and 423106, respectively). Data were acquired using a BD Fortessa X20 or Beckman Coulter CytoFLEX LX, and analyses were performed using FlowJo V10 software.

Isolation of Human and Murine NK Cells

Human NK cells from healthy individuals (leukoreduction collars) were isolated by negative selection using the EasySep™ Human NK Cell Isolation Kit (Stem Cell Technologies, catalog number 17955), which resulted in NK cell purities of at least 90%. Leukoreduction collars were provided in an anonymous manner by Brigham and Women's Hospital (Boston, USA). NK cells were expanded in vitro in G-Rex 6-wells plates (Wilson Wolf, catalog number 80240M) using RPMI-1640 media supplemented with 10% FBS, 5% human AB serum, 1,000 U/ml IL-2, and 20 ng/ml IL-15 (both cytokines were from BD Biosciences); media was replenished once per week until NK cells were used for experiments.

Murine NK cells were isolated by meshing spleen tissue using a 70 m cell strainer, followed by red cell lysis (ACK buffer) and staining with PE-conjugated anti-mouse CD49b mAb (Biolegend, catalog number 108908) and APC-conjugated anti-mouse CD3ε mAb (Biolegend, catalog number 100312). NK cells were sorted by flow cytometry, yielding typical purities of ˜99%. These cells were immediately injected in Rag2^{-/-} Il2rg^{-/-} KO mice for experiments involving allogeneic or syngeneic NK cells.

NK Cell-Mediated Killing Assays

For long-term NK cell-mediated killing assays, GFP⁺ control and B2M-KO A375 cells were pretreated for 24 hours with MICA/B or isotype control mAbs (20 μg/ml) in tissue culture media. Subsequently, tumor cells were detached with Versene, washed with PBS, and plated in black-wall 96-well plates (Corning™, catalog number 3603) at a density of 5×10³ cells per well. Human NK cells were added 1-2 hours later at different effector to target ratios as indicated in the figures, and IL-2 (300 U/ml, BD Biosciences) was added to support NK cell survival. The number of GFP⁺ tumor cells was tracked over time using a Celigo Image Cytometer (Nexcelom Bioscience, Lawrence, USA), as reported previously (33).

For short-term NK cell killing assays, A375 melanoma cells were pretreated for 24 hours with MICA/B or isotype control mAbs (20 μg/ml) in tissue culture media and then used as target cells in 4-hour ⁵¹Cr-release assays, as described previously (23). NK cells were isolated by negative selection from leukapheresis reduction collars and cultured for 24 hours with 1,000 U/ml IL-2 (BD Biosciences) in 96-wells U-bottom plates prior to use in the assay. KIR receptors on NK cells were blocked in the ⁵¹Cr-release assay by addition of isotype control mAb or anti-KIR2DL2/3 plus anti-KIR2DL4 mAbs (BioLegend, catalog numbers 312602 and 347003).

CD8 T Cell Cytotoxicity Assay

To confirm resistance of Jak1-KO and B2m-KO B16F10-MICA cell lines to CD8 T cell-mediated cytotoxicity, a Celigo based image cytometry assay was performed (33). Briefly, tumor cells were pulsed with 10 nM Ova peptide overnight, washed with PBS and added to 96-well plates (5,000 tumor cells per well). The tumor cells were co-cultured with naïve OT-I CD8 T cells at different effector to target ratios (1:0 no T cells; 1:1, 2:1 and 5:1; 8-10 replicates per group); 48 hours later, supernatants were removed, and wells were washed with PBS to remove dead tumor cells and CD8 T cells. The plate was then analyzed using the Celigo instrument for quantification of live tumor cells.

Bulk RNA-Seq Analysis of Human A375 Melanoma Cells

Parental A375 cells were treated for 24 hours with panobinostat (50 nM) or the corresponding volume of PBS. Cells were then detached with Versene, and RNA was isolated with RNeasy Plus Mini Kit and RNase-Free DNase Set, respectively (both Qiagen kits, catalog numbers 74134 and 79254, respectively). Generation of cDNA, sequencing, and analyses were done as previously reported (23).

Real-Time Quantitative PCR (qPCR)

A375 cells were treated for 24 hours with panobinostat (50 nM). Subsequently, cells were washed twice with PBS, pelleted and used for extraction of total RNA using the RNeasy mini kit (#74106, Qiagen) according to the manufacturer's protocol. One microgram of the extracted RNA was used to synthesize cDNA using SuperScript IV VILO Master Mix (ThermoFisher, 11756050). Diluted cDNA was used for qPCR using TaqMan Gene Expression MasterMix (Life Technologies, 4369016), TaqMan probes (MICA—Hs00741286_m1, MICB—Hs00792952_m1, GAPDH—Hs02786624_g1, ULBP2—Hs00607609_mH, and RAETIL—Hs04194671_s1) and QuantStudio 6 Flex Real-Time PCR System (ThermoFisher). To examine changes in gene expression between groups, ΔΔCT values were determined from mean CT values of three technical replicates per sample in each group. Fold change in gene expression was represented relative to GAPDH (a housekeeping gene) for each sample.

Mice

Wild Type (WT) C57BL6/J, Ighm^{-/-} C57BL6/J, CB6F1/J and NSG mice were purchased from the Jackson Laboratories (catalog numbers 000664, 002288, 100007, and 005557, respectively). Rag2^{-/-} 12rg^{-/-} knockout mice were purchased from Taconic (catalog number 4111). Mice were male (except for NSG mice that were female) and 6-8 weeks of age. Mice were housed in the vivarium of the Dana-Farber Cancer Institute and Icahn School of Medicine at Mount Sinai. The institutional committees for animal use approved the procedures used in this study.

Metastasis Models in Immunocompetent Mice

B16F10-MICA tumor cells (control, B2m-KO or Jak1-KO) were inoculated intravenously into C57BL/6 mice (WT or Ighm^{-/-}) via the tail vein (1 to 7×10⁵ cells in 100 μl of PBS depending on the experiment, as described in figure legends). Treatment was initiated in Ighm^{-/-} mice when mice had established metastases (day 7 following tumor inoculation) by intraperitoneal injection of isotype control mAb (BioXcell, catalog number BE0085) or 7C6-mIgG2a mAb (200 μg per injection, days 7, 8 and then once per week). In an alternative protocol, WT mice (Ighm^{+/+}) received antibody injections on days 1, 2 and then once per week. Antibodies that induced depletion of CD8 T cells (100 μg anti-CD8β, BioXcell, catalog BE0223) or NK cells (1:10 dilution anti-asialo GM1, Wako Chemicals, catalog 986-10001, and 100 μg anti-NK1.1, clone PK136, BioXcell) were injected on days −1, 0 and then once per week relative to tumor cell inoculation; murine IgG1 was used as control IgG (BioXcell, catalog BE0083). Lung metastases were quantified on day 14 under a stereomicroscope following formalin fixation of the tissue. Alternatively, the survival of mice was recorded.

For the LLC1-MICA metastasis model, WT C57BL/6 mice were inoculated intravenously via the tail vein with 1.0 to 1.5×10⁶ tumor cells (as indicated in the figure legends) in 0.1 ml of PBS. 7C6-mIgG2a or isotype control mAbs (200 μg) were administered on days 2, 3 and then once per week relative to tumor cell inoculation. For experiments involving adoptive transfer of NK cells, 2×10⁵ NK cells isolated from WT C57BL/6 (syngeneic) or CB6F1/J (allogeneic) mice were injected intravenously into Rag2^{-/-} Il2rg^{-/-} knockout mice. These NK cells were isolated by flow cytometry as CD3ε⁻ CD49b⁺ cells. LLC1-MICA tumor cells (7×10⁵ cells) were injected intravenously one day following NK cell transfer. 7C6-mIgG2a or isotype control mAbs (200 μg/injection) were given intraperitoneally on days 2, 3 and then once per week. On day 14, mice (WT or Rag2^{-/-} Il2rg^{-/-} knockout) were euthanized by CO₂ inhalation, and Indian ink (30%) was injected into the trachea to enable counting of lung metastases, as previously described (34). Lung tissue was treated using Fekete's fixative and surface metastases were counted using a stereomicroscope.

Characterization of Murine NK Cells in the B16F10-MICA Metastasis Model

WT C57BL/6 mice were inoculated intravenously with 7×10⁵ B16F10-MICA cells that either had the control, B2m-KO or Jak1-KO genotype. Mice were treated with 7C6-mIgG2a or isotype control mAbs (200 μg/injection) on days 1 and 3 following tumor cell inoculation. On day 12, mice were injected intravenously with 50 μl of APC-conjugated anti-mouse CD45.2 (Biolegend, 109814) to label intravascular immune cells and then euthanized. Lung tissue was cut into small pieces, resuspended in RPMI-1640 supplemented with 1 mg/ml collagenase type IV, 0.1 mg/ml hyaluronidase and 20 U/ml DNase, and processed using a gentleMACS instrument (Miltenyi). The cell suspension was then incubated with mouse TruStain FcX™ (Biolegend, catalog number 101320) and multiple antibodies, including PE-Cy7-conjugated anti-mouse CD45.2 (Biolegend, 109830), APC-conjugated anti-mouse CD3s (Biolegend, 100312), APC-conjugated anti-mouse TCRβ (Biolegend, 109212), BV785-conjugated anti-mouse NK1.1 (BD Biosciences, 740853), PE-CF594-conjugated anti-mouse CD49b (BD Biosciences, 562453), Alexa488-conjugated anti-mouse EOMES (Invitrogen, 53-4875-82), PE-conjugated anti-mouse GZMA (Invitrogen, 12-5831-82), BV421-conjugated anti-mouse NKG2D (BD Biosciences, 562800), PERCP-CY5.5-conjugated anti-mouse CD16/32 (Biolegend, 101324), BV510-conjugated anti-mouse Ly49C/I (BD Biosciences, 744028) and Zombie UV. Cells were analyzed using a CytoFLEX Flow Cytometer (Beckman Coulter), and data were processed using FlowJo V10.

Humanized Mouse Model

NSG mice were reconstituted intravenously with human NK cells (1 to 2×10⁶ cells) that had been expanded in vitro as described above; IL-2 (Peprotech, catalog number 200-02) was injected intraperitoneally (7.5×10⁴ units) to support in vivo survival of NK cells, as previously reported (23). A375 melanoma cells (5×10⁵ cells, control or B2M-KO) were injected one day later (day 0). One day after tumor cell inoculation, mice received another dose of IL-2, plus isotype control (BioXcell, catalog BE0096) or 7C6-hIgG1 mAbs (200 μg) as well as PBS or 10 mg/kg panobinostat (ApexBio, catalog number A8178). On day 2, mice were again reconstituted with human NK cells from the same donor and also received injections of IL-2, antibodies as well as PBS or panobinostat. Metastases were quantified 2 weeks after the last treatment as described above for the LLC1-MICA metastasis model (injection of Indian ink into the trachea, treatment of lung tissue with Fekete's fixative).

NSG mice were inoculated with 1×10⁶ ZsGreen⁺ A375 melanoma cells to study the effect of MICA/B mAb and panobinostat treatment on MICA/B surface levels in lung metastases; these mice did not receive human NK cells. When metastases were established (two weeks later), mice were treated on two subsequent days with 7C6-hIgG1 or isotype control mAbs (200 μg) as well as panobinostat (10 mg/kg) or PBS as a solvent control. One day following the last treatment, mice were euthanized by CO₂ inhalation, and lung tissue was dissociated mechanically to preserve the integrity of MICA/B proteins. Tumor cells were identified as viable large cells that were ZsGreen positive but negative for the murine CD45 antigen. MICA/B surface protein was labeled with 6D4-PE mAb (Biolegend, catalog 320906) and quantified by flow cytometry.

Characterization of Human NK Cells in Tumor-Free NSG Mice

Tumor-free NSG mice were inoculated intravenously with 2×10⁶ human NK cells that were expanded in vitro as described above. At the same time, mice also received intraperitoneal injections of IL-2 (7.5×10⁴, Peprotech) as well as panobinostat (10 mg/kg) or PBS as the solvent control. One day later, blood was collected via eye bleeding, and human NK cells were analyzed by flow cytometry.

Statistical Analyses

All statistical analyses were performed using GraphPad Prism 8 software, and the relevant statistical tests are indicated in each figure legend.

Results

NK Cell-Mediated Killing of Human B2M-Deficient Melanoma Cells is Enhanced by MICA mAb

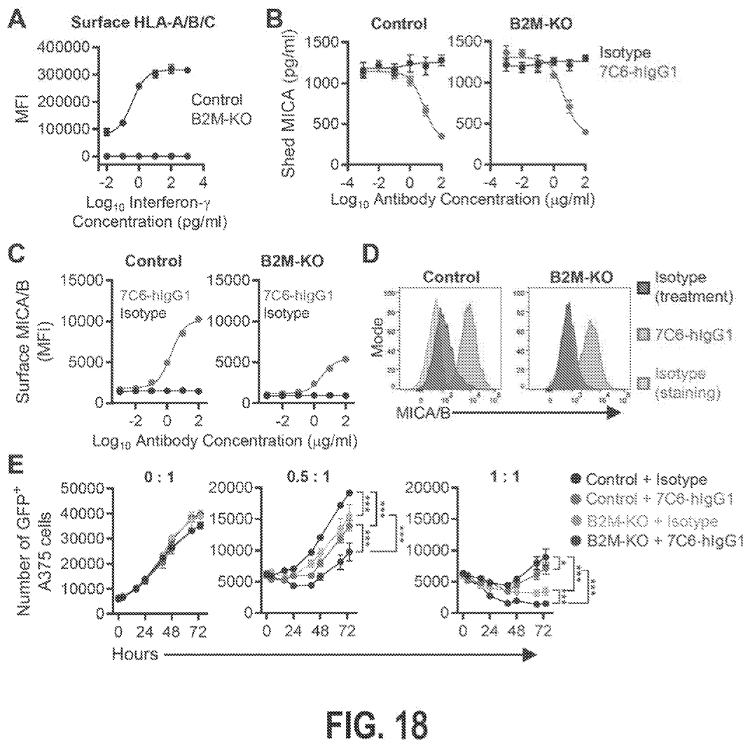

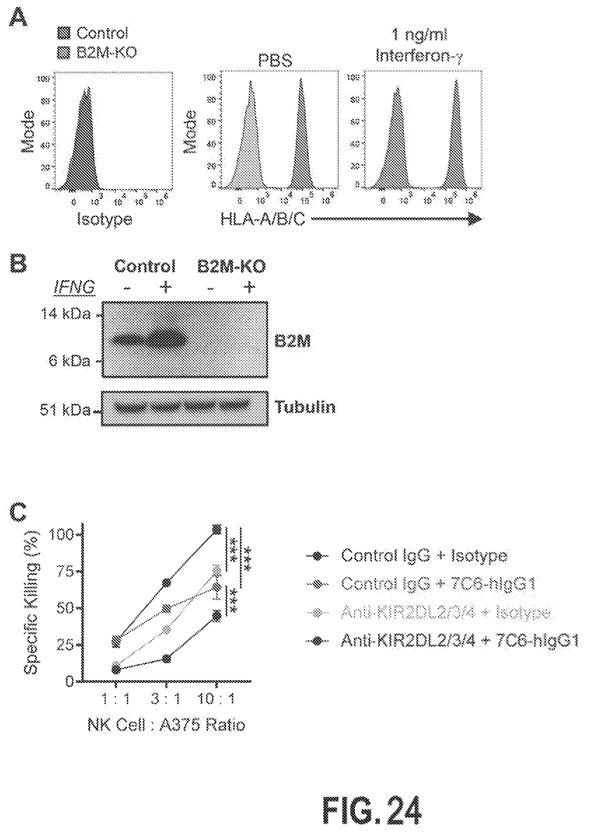

The effect of MICA/B α3 domain specific antibody on NK cell-mediated immunity against human B2M-deficient tumor cells was examined. We inactivated the B2M gene in human A375 melanoma cells which resulted in a complete loss of MHC-I surface proteins even following stimulation with IFNγ (FIG. 18A, FIG. 24A-B). B2M deficiency did not abolish MICA/B expression, although we noted a ˜50% decrease of cell surface levels. Both B2M-KO and control A375 melanoma cell lines shed MICA into the supernatant (FIG. 18B-D). Treatment with a MICA/B α3 domain specific mAb (7C6-hIgG1) inhibited MICA shedding and increased surface levels of MICA/B proteins for both control and B2M-deficient A375 cells (FIG. 18B-D).

NK cells express inhibitory receptors for MHC-I molecules (12), and without wishing to be bound by theory, B2M-deficient tumor cells can be more sensitive to MICA/B mAb treatment. We studied the kinetics of NK cell-mediated killing of human A375 melanoma cells using an imaging-based system that enabled counting of fluorescent tumor cells in 96-well plates at multiple time points. This technique enables investigation of NK cell—tumor cell interactions at low effector to target ratios that are relevant to the tumor microenvironment (33). We used primary NK cells isolated via negative selection from the blood of healthy donors for these experiments. This experiment demonstrated that MICA/B mAb treatment (7C6-hIgG1) was more effective against B2M-KO compared to control A375 melanoma cells. Even at a low effector to target ratio (1:1), only a small number of fluorescent B2M-KO melanoma cells remained at late time points (48-72 hours) in the presence of the MICA/B mAb (FIG. 18E). We previously established that the 7C6 mAb induced dual engagement of NKG2D and CD16a receptors in human NK cells, and that both receptors contributed to NK cell-mediated killing of target cells (23). KIR2DL2, KIR2DL3, and KIR2DL4 are some of the best characterized inhibitory receptors for MHC-I molecules on human NK cells (12). Although NK cells from healthy donors are alloreactive to A375 cells due to MHC mismatch, antibody-mediated blockade of those receptors increased NK cell-mediated killing of 7C6-hIgG1-treated A375 melanoma cells (Supplementary FIG. 18C), therefore NK cell inhibitory receptors recognized MHC-I protein on A375 cells. Altogether, these experiments demonstrated that loss of MHC class I surface expression rendered human tumor cells more vulnerable to NK cells, which was further enhanced by the presence of a MICA/B mAb.

MICA/B Antibody Induced Immunity Against Metastases Resistant to Cytotoxic T Cells

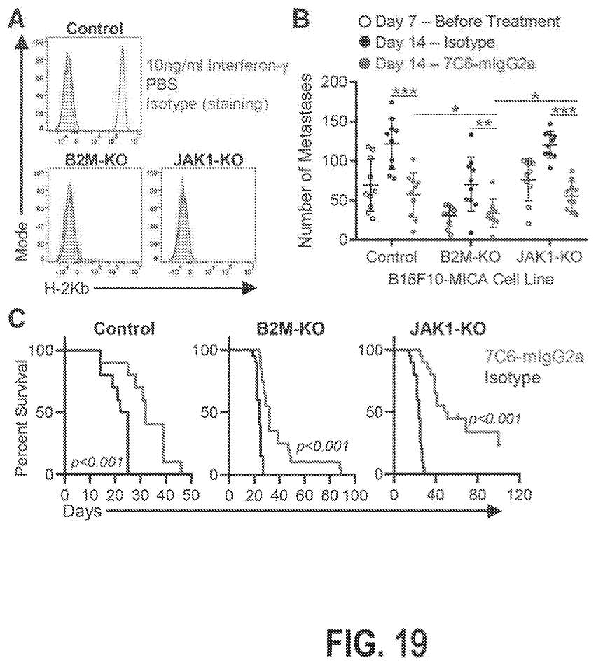

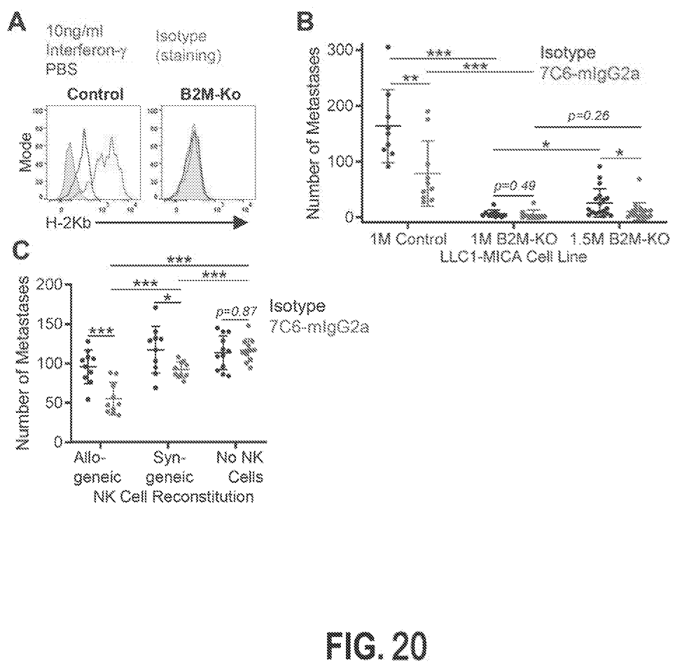

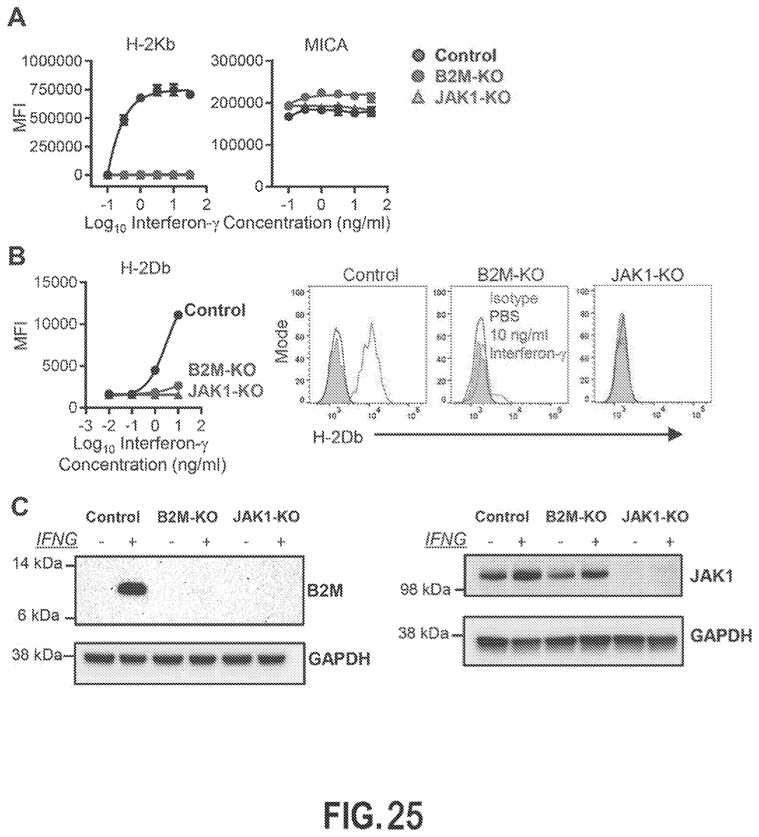

We used two murine models to validate whether MICA/B mAb treatment can induce immunity against tumors with inactivating mutations in the MHC-I and IFNγ pathways (B2m and Jak1 mutations, respectively). The Jak1 mutation was of interest because IFNγ is secreted by both T cells and NK cells. IFNγ signaling in tumor cells not only enhances expression of many genes of the MHC class I pathway, but also inhibits tumor cell proliferation (3). Therefore, Jak1 mutations can either negatively impact the ability of NK cells to control tumor cell growth or enhance NK cell activation through loss of MHC class I proteins that engage inhibitory receptors on NK cells. B16F10 melanoma and LLC1 lung cancer cell lines were transduced with a lentiviral vector to induce expression of human MICA which is known to bind to the murine NKG2D receptor (23). These murine models had differences in their pattern of MHC class I expression. B16F10 melanoma cells had a very low basal surface level of H-2K^{b} and H-2D^{b} proteins, but exposure to IFNγ resulted in a striking increase of H-2K^{b} and H-2D^{b} surface proteins (FIG. 19A, FIG. 25A-B). In contrast, LLC1 lung tumor cells had detectable basal levels of H-2K^{b} but not H-2D^{b}; the surface expression of H-2K^{b} was increased by IFNγ treatment (FIG. 20A, FIG. 274A).

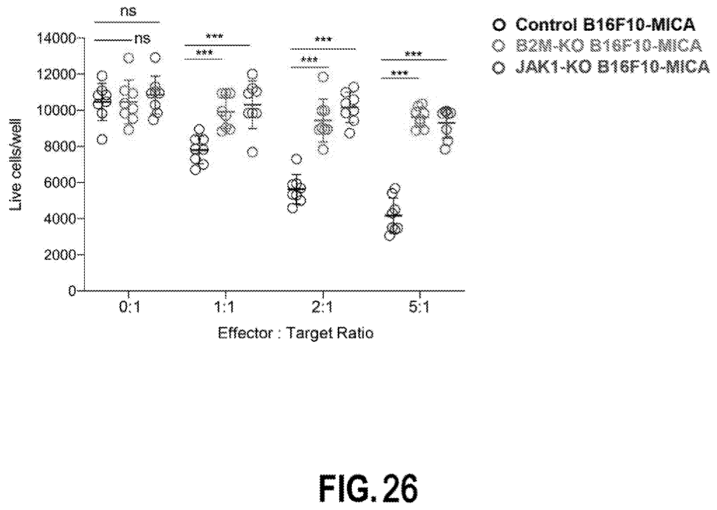

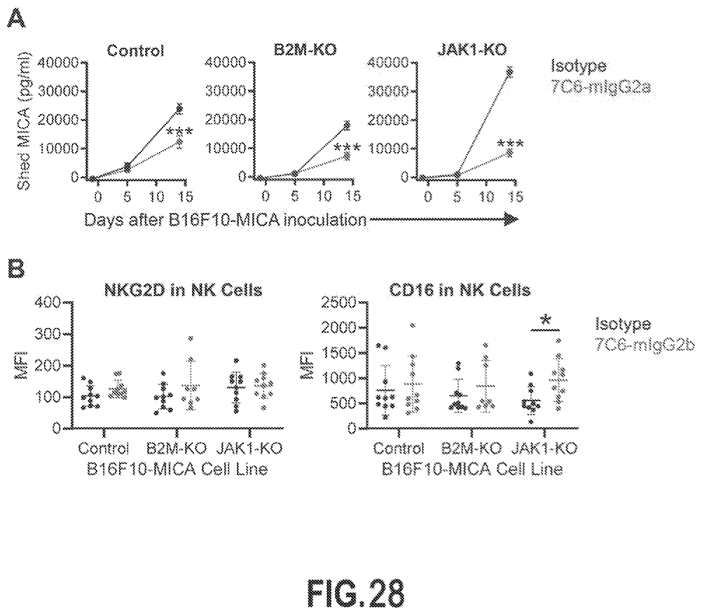

We inactivated B2m or Jak1 genes in B16F10-MICA melanoma cells (FIG. 19A, FIG. 25A-C), which caused resistance to CD8 T cell-mediated killing (FIG. 26), and tested the efficacy of MICA/B mAb treatment in a lung metastasis model. Edited tumor cells were injected intravenously, and treatment was initiated on day 7 when established surface lung metastases were detected (as determined by pathological analysis of a subset of mice, labeled as ‘before treatment’) (FIG. 19B). B cell deficient Ighm^{-/-} mice were used as hosts to prevent development of endogenous antibodies against human MICA, as previously reported (23). Treatment with the MICA/B mAb (7C6-mIgG2a) inhibited the outgrowth of lung metastases by control, B2m-KO and Jak1-KO B16F10-MICA cells (FIG. 19B). MICA/B mAb treatment also reduced plasma levels of shed MICA (FIG. 28A). In a separate set of experiments, we also analyzed the survival of wild-type (Ighm^{+/+}) mice inoculated with B16F10-MICA cell lines and treated with MICA/B or control mAbs. In these experiments, antibodies were administered on days 1 and 2 relative to B16F10-MICA inoculation, which was earlier than the generation of endogenous MICA antibodies by the murine immune system. 7C6 compared to isotype control antibody treatment significantly increased survival of WT mice with control, B2m-KO or Jak1-KO melanoma metastases (FIG. 19C).

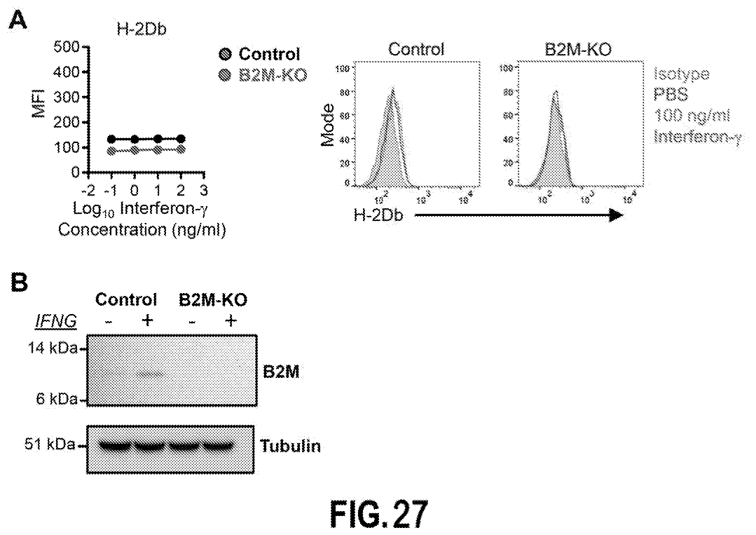

We also examined the efficacy of MICA/B antibody treatment in the LLC1-MICA tumor model. We knocked out the B2m gene in this cell line (FIG. 27B). Control LLC1 cells expressed H-2K^{b} at baseline and treatment with IFNγ increased MHC-I surface protein levels, whereas B2m-KO LLC1 cells had no MHC-I expression even following treatment with IFNγ (FIG. 20A). Tumor cells were injected intravenously into WT mice, and mAb treatment was initiated on day 2. MICA/B mAb treatment reduced the number of lung metastases formed by control LLC1-MICA tumor cells. Inactivation of the B2m gene reduced the number of lung metastases compared to control LLC1-MICA cells to almost undetectable levels. We therefore increased the number of inoculated tumor cells by 50% which resulted in formation of lung metastases by B2m-KO LLC1-MICA cells. Under these experimental conditions, we observed a significant reduction in the number of B2m-KO LLC1-MICA metastases following treatment with 7C6-mIgG2a compared to isotype control mAb (FIG. 20B).

NK cell inhibitory receptors for MHC-I are encoded by polymorphic genes and play a role for the generation of self-tolerant NK cells, which are a population of NK cells that are inhibited upon recognition of self MHC-I on target cells. These inhibitory receptors also have specificity for polymorphic variants of MHC-I proteins (35). C57BL/6 mice and Balbc mice differ in their MHC-I polymorphic variants and as consequence the F1 mice from the cross between these two mouse strains (called CB6F1) have a population of NK cells that is not tolerant to the MHC-I variants from the C57BL/6 strain (36). Such alloreactive NK cells are key to the therapeutic efficacy of allogeneic stem cell transplantation for leukemia (37). We took advantage of this mouse strain to further confirm the role of NK cells and inhibitory receptors for MHC-I proteins using an adoptive transfer model. Of note, the LLC1 cell line is syngeneic to C57BL/6 mice. Rag2^{-/-} Il2rg^{-/-} KO mice were reconstituted with either syngeneic NK cells (from C57BL/6 mice) or allogeneic NK cells (from CB6F1 mice). Both syngeneic and allogeneic NK cells reduced the number of lung metastases formed by LLC1-MICA tumor cells when mice were treated with MICA/B versus isotype control mAb. Also, MICA/B antibody treatment was more effective when allogeneic NK cells were transferred (FIG. 20C). One advantage of this model is that it does not affect NKG2A recognition of non-classical MHC-I molecules because surface expression of Qa-1 (a non-classical MHC-I molecule) also requires association with B2M; therefore, B2M-deficient tumors are not recognized by NK cell inhibitory receptors for both classical and non-classical MHC-I molecules (12, 38). The engagement of MHC-I proteins by inhibitory receptors on NK cells reduces the efficacy of anti-tumor immunity induced by the MICA/B mAb.

Essential Role of NK Cells for Efficacy of MICA/B Antibody

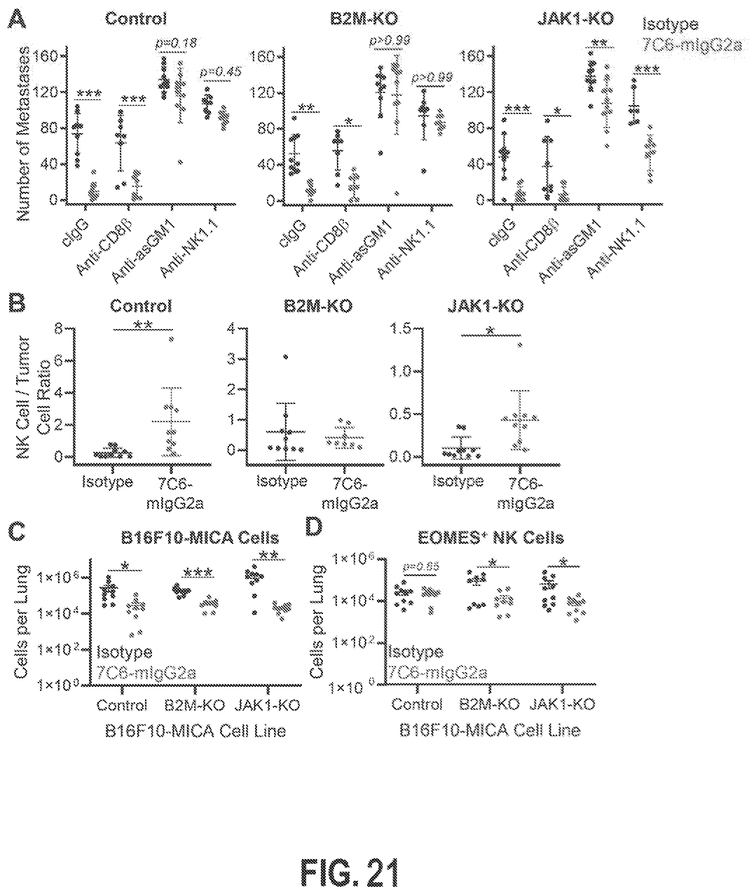

We next performed mechanistic experiments with the B16F10-MICA cell lines inoculated into WT mice. Depletion of NK cells, but not of CD8 T cells, resulted in a complete loss of MICA/B mAb efficacy against both control and B2m-KO B16F10-MICA tumor cells, whereas NK cell depletion greatly reduced MICA/B mAb efficacy against Jak1-KO B16F10-MICA tumor cells (FIG. 21A). We also examined lung-infiltrating NK cells by flow cytometry which were distinguished from blood NK cells by intravenous injection of an APC-conjugated anti-CD45.2 antibody prior to euthanasia, as reported previously (23). MICA/B mAb treatment increased the degree of NK cell infiltration into control or Jak1-KO B16F10-MICA tumors (FIG. 21B). In this analysis, NK cell infiltration was normalized to tumor burden because the number of B16F10-MICA tumor cells was substantially reduced in MICA/B mAb treated mice (FIG. 21C-D). We did not observe significant differences in NKG2D and CD16 expression by tissue-infiltrating NK cells depending on the genotype of B16F10-MICA melanoma cells, except for an increase in CD16 levels for NK cells in the Jak1-KO B16F10-MICA lung metastasis model following treatment with 7C6-mIgG2a mAb (FIG. 28B). These data demonstrate that MICA/B mAb treatment inhibits the outgrowth of melanoma metastases in a NK cell-dependent manner even when tumor cells carry inactivating mutations in B2m or Jak1 genes.

Enhanced MICA/B Surface Protein Levels on Human Tumor Cells Treated with the Combination of MICA/B mAb and HDAC Inhibitor

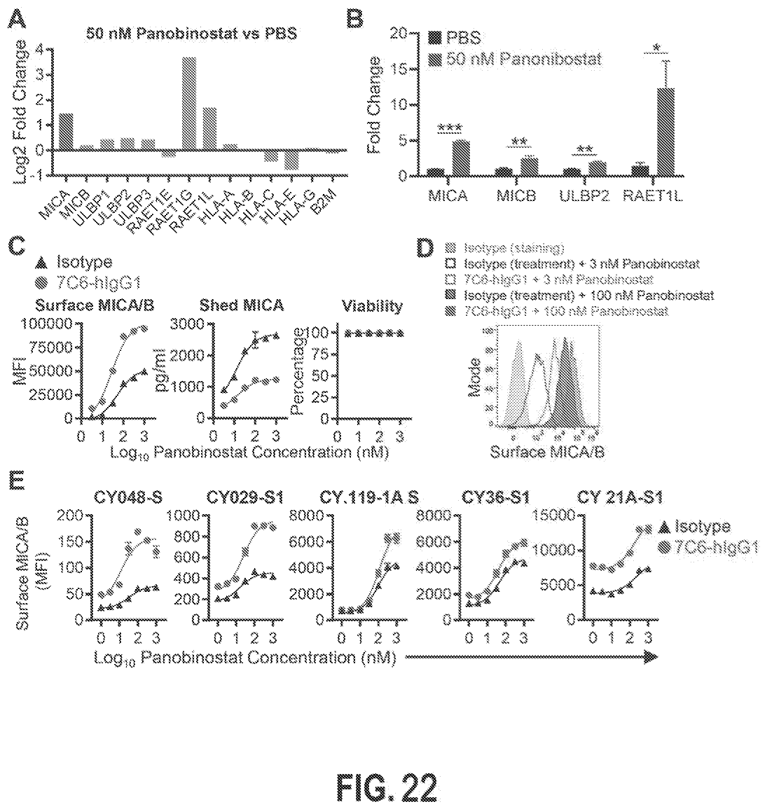

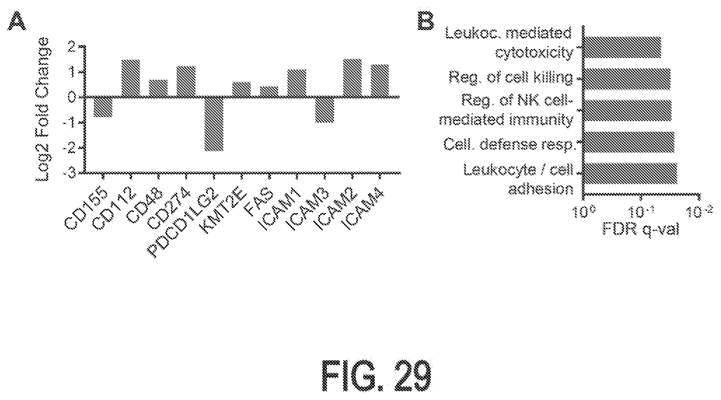

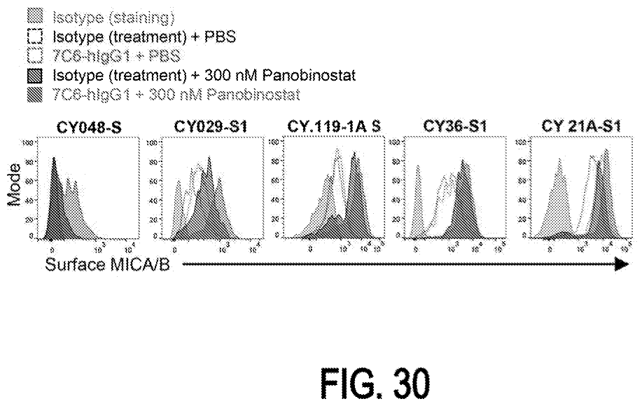

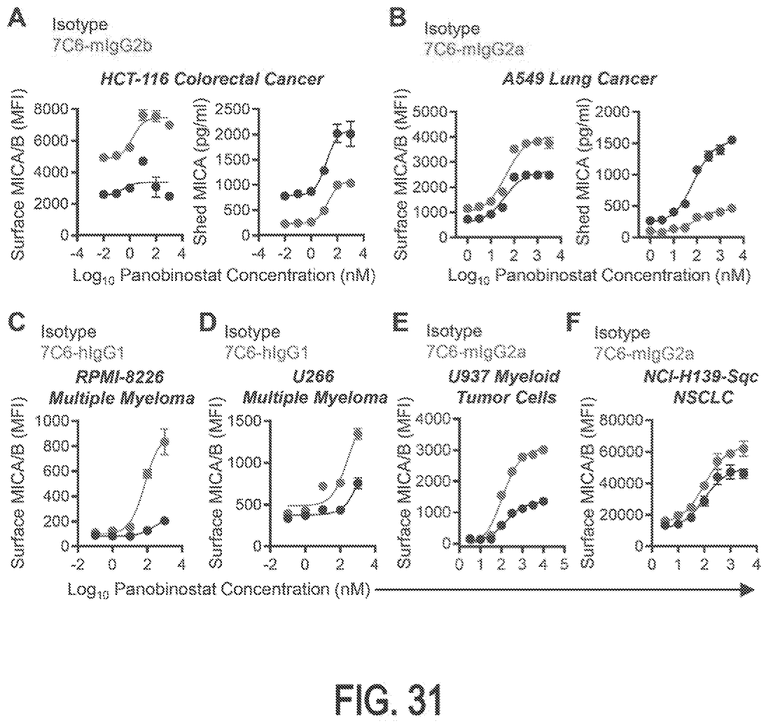

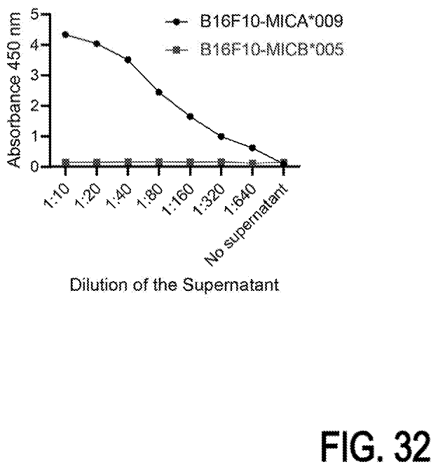

In the tumor models described herein, MICA transcription was controlled by a heterologous promoter that induced high levels of MICA, as previously shown (23). However, in human cancers MICA/B expression is induced in response to DNA damage and cellular stress (13). The transcription of MICA and MICB genes is epigenetically regulated by HDACs, and that HDAC inhibitors enhance transcription of these genes (27, 28). The pan-HDAC inhibitor panobinostat was approved by the U.S. Food and Drug Administration (FDA) for the treatment of multiple myeloma (39). Previous work in multiple mouse models of cancer established that an intact immune system is required for the therapeutic activity of panobinostat (40). We therefore examined whether the combination of panobinostat and 7C6-hIgG1 mAb could enhance MICA/B protein levels by increased transcription of MICA B genes (panobinostat) and stabilization of the encoded protein on the cell surface (MICA/B mAb). RNA-seq analysis demonstrated that treatment of A375 melanoma cells with panobinostat (50 nM) for 24 hours increased mRNA levels of multiple genes encoding NKG2D ligands, including MICA, RAET1G, and RAET1L. However, panobinostat did not increase mRNA levels of genes encoding classical or non-classical MHC-I molecules (FIG. 22A). We also confirmed by real-time qPCR that panobinostat increased the expression of MICA and RAET1L; this assay also detected an increase in MICB and ULBP2 (FIG. 22B). Panobinostat also affected transcription of many other genes in A375 melanoma cells, some of which represented immunity-related pathways (FIG. 29A-B). Surface MICA/B protein levels were substantially increased by the combination of panobinostat and MICA/B mAb, and the concentration of shed MICA was diminished without a reduction in cellular viability (FIG. 5C-D). We also examined MICA/B protein levels by a panel of short-term human melanoma cell lines established from metastatic lesions (23, 31). Treatment with panobinostat plus MICA/B mAb substantially increased the surface density of MICA/B proteins compared to treatment with individual compounds (FIG. 22E, FIG. 30). These conclusions were further supported by analysis of a panel of human tumor cell lines (FIG. 31A-F). Of note, shed MICB was not analyzed because the ELISA was specific for MICA (FIG. 32). These data demonstrate that combinatorial approaches which increase transcription of MICA B genes and stabilize synthesized proteins result in a substantial increase of surface MICA/B proteins on human cancer cells.

Efficacy of MICA/B mAb and Panobinostat Combination Therapy in a Humanized Mouse Model

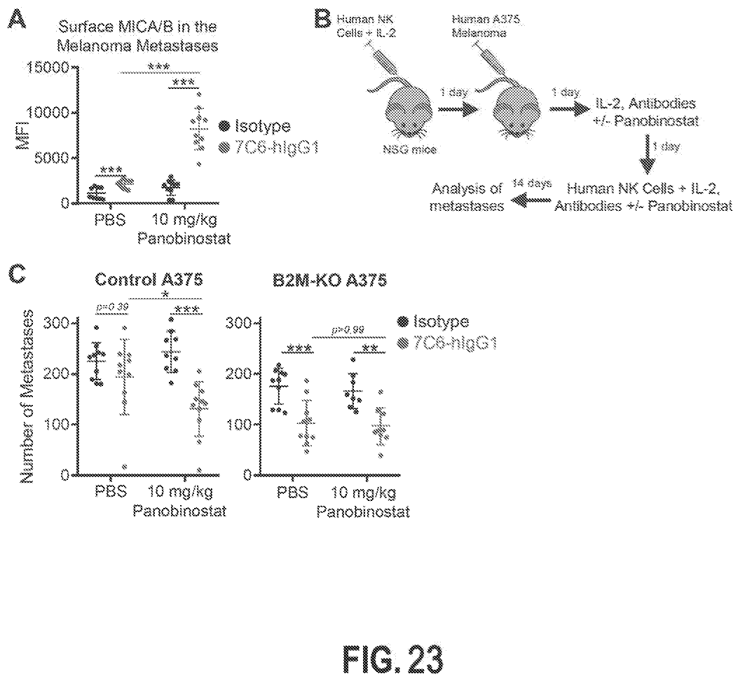

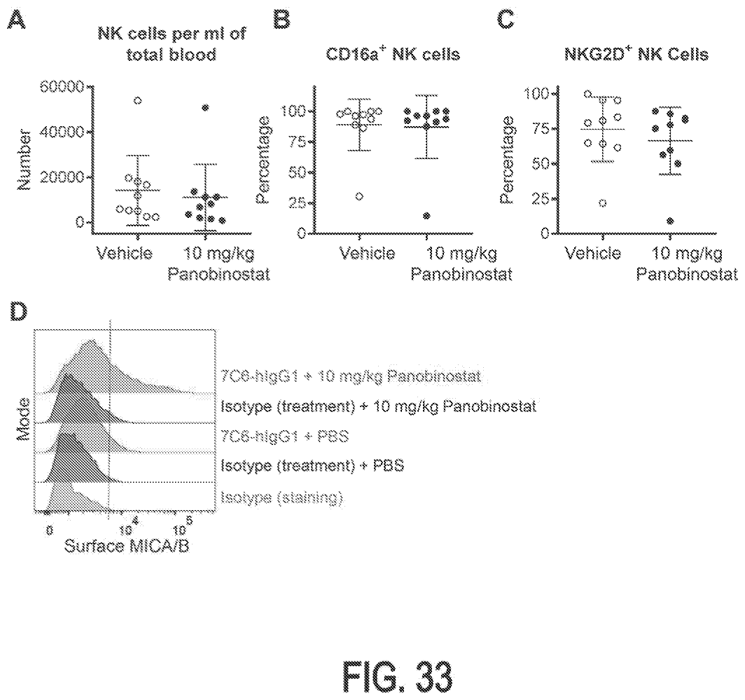

We next investigated the in vivo activity of panobinostat on surface MICA/B protein levels on human melanoma cells. We selected a dose of panobinostat (10 mg/kg) based on a previous research article that established the efficacy of panobinostat for multiple myeloma treatment in mouse models (41). We first established that the selected dose of panobinostat did not negatively impact human NK cells transferred to immunodeficient NSG mice (based on number of circulating total NK cells as well as percentage of CD16a or NKG2D positive NK cells, FIG. 33A-C). Next, we injected ZsGreen⁺ A375 melanoma cells intravenously into NSG mice and waited for two weeks until metastases were established. Mice were then treated twice at a 24-hour interval with panobinostat (or PBS), MICA/B mAb (or isotype control mAb) or the combination of panobinostat plus MICA/B mAb (or panobinostat plus isotype control mAb). One day later, MICA/B surface protein levels were quantified on ZsGreen⁺ tumor cells from dissociated lung tissue by flow cytometry. The selected dose of panobinostat did not significantly increase MICA/B protein levels on melanoma cells as a monotherapy, but the combination of panobinostat and MICA/B mAb resulted in high MICA/B surface levels on ZsGreen⁺ A375 melanoma cells in pulmonary metastases (FIG. 23A, FIG. 33D).

Based on our prior experience, survival of transferred human NK cells was limited in NSG mice and only a relatively small number of human NK cells infiltrated lung tissue. We therefore initiated treatment one day following inoculation of human A375 melanoma cells (FIG. 23B). The early start of treatment enabled NK cell recognition of tumor cells that had not yet infiltrated deeply into the lung tissue. We found that only the combination of panobinostat plus MICA/B mAb reduced the number of lung metastases formed by control (B2M-WT) A375 melanoma cells, while monotherapy with either panobinostat or MICA/B mAb was ineffective. In contrast, monotherapy with the MICA/B mAb significantly reduced the number of lung metastases formed by B2M-KO A375 melanoma cells (FIG. 23C). Without wishing to be bound by theory, the combination of MICA/B mAb and panobinostat did not enhance this effect against B2M-KO metastases, because NK cell reconstitution was limited in this model. These results demonstrate that MICA/B mAb treatment is more effective against MHC-I deficient human melanoma metastases in this humanized mouse model, whereas the combination therapy is effective against melanoma metastases that express MHC-I proteins.

Discussion

Primary and secondary resistance to checkpoint blockade are issues in oncology. Many mechanisms of resistance to checkpoint blockade are related to the MHC-I and IFNγ signaling pathways in tumor cells. These include mutations of B2M or other genes in the MHC-I antigen presentation pathway, transcriptional and epigenetic silencing of neoantigen or MHC-I expression as well as inactivating mutations in the IFNγ signaling pathway (4-6). Although MICA/B proteins have a similar overall structure to MHC-I proteins, they do not assemble with β2-microglobulin (24). Also, transcription of the MICA B genes is induced by DNA damage and cellular stress rather than by IFNγ (13). Therefore, inactivating mutations in the MHC-I and IFNγ pathway do not abrogate MICA/B expression.

The loss of MHC-I expression removes an inhibitory signal for NK cells, but sufficient activating signals are also required for induction of NK cell-mediated tumor immunity (12). We show that metastases with inactivating mutations in the MHC-I (B2M mutation) or IFNγ signaling (JAK1 mutation) pathways can be treated with a MICA/B α3 domain specific antibody. This antibody inhibits proteolytic shedding of MICA/B, a common evasion mechanism from NKG2D receptor-mediated immunity in human cancers. We previously showed that treatment with this mAb induces activation of both NKG2D (increased density of MICA/B ligand) and CD16a (Fc region of mAb) receptors on NK cells and that tumor immunity elicited by this mAb is NK cell dependent (23). Without wishing to be bound by theory, two approaches can elicit NK cell-mediated tumor immunity with such a mAb. First, a MICA/B α3 domain specific mAb could be used to treat tumors resistant to checkpoint blockade due to inactivating mutations in the MHC-I or IFNγ signaling pathways. Second, simultaneous administration of a MICA/B mAb and a PD-1 mAb can activate both NK cells and CD8 T cells and thereby prevent the outgrowth of tumor clones resistant to cytotoxic T cells. Such an approach can be of interest for advanced human tumors with extensive heterogeneity. The NKG2D receptor is also expressed by human CD8 T cells, γδ T cells and ILCs (14, 42, 43, 44). MICA/B mAb treatment therefore, without wishing to be bound by theory, can enhance T cell-mediated tumor immunity via the NKG2D receptor expressed by CD8 T cells. The shedding of NKG2D ligands is not always a mechanism of immune escape. For example, the shedding of MULT-1, a murine NKG2D ligand, promotes the NK cell-mediated immunity (45).

Many therapeutic approaches used in oncology enhance expression of MICA/B proteins by tumor cells. For example, HDAC inhibitors enhance transcription of MICA/B genes, such as panobinostat, a FDA approved drug (27, 28, 39). However, proteolytic shedding of MICA/B proteins by tumor cells limits the effect of such drugs on NKG2D receptor activation. We show that the combination of panobinostat and a MICA/B α3 domain antibody greatly increased MICA/B surface protein levels on tumor cells in vivo and enhanced NK cell-mediated immunity against melanoma metastases. Without wishing to be bound by theory, the HDAC inhibitor can also induce expression of NKG2D ligands in healthy tissues. A similar approach can be used to develop combination therapies with other FDA approved drugs. The DNA damage response induced by radiation therapy strongly enhances MICA/B transcription (46). A combination of local radiotherapy and systemic immunotherapy with a MICA/B mAb can limit immune-related adverse events that have been observed with combinations involving two systemic immunotherapy agents (such as PD-1 and CTLA-4 mAbs). Also, there is clinical evidence that radiation therapy in combination with checkpoint blockade (CTLA-4 blockade) can induce systemic tumor immunity against non-irradiated lesions (abscopal effect) (47).

In summary, we show that metastases with mutations that cause resistance to cytotoxic T cells can be targeted by NK cells when MICA/B shedding is inhibited with a mAb. A number of combination strategies can further enhance the activity of NK cells against metastatic lesions:

1. Approaches that enhance MICA/B protein expression by tumor cells (such as panobinostat, local radiation therapy) (46),
2. Cytokines that enhance NK cell function within tumors and reduce TGFβ-mediated NKG2D downregulation (such as the IL-15/IL-15Rα complex) (48),
3. Antibodies that target inhibitory receptors on NK cells (49).
Induction of NK cell-mediated immunity can thus provide a strategy to treat tumors with escape mutations from T cell-mediated cytotoxicity.

### REFERENCES CITED IN THIS EXAMPLE

1. A. Ribas, J. D. Wolchok, Cancer immunotherapy using checkpoint blockade. Science 359, 1350-1355 (2018).
2. T. N. Schumacher, R. D. Schreiber, Neoantigens in cancer immunotherapy. Science 348, 69-74 (2015).
3. B. S. Parker, J. Rautela, P. J. Hertzog, Antitumour actions of interferons: implications for cancer therapy. Nat Rev Cancer 16, 131-144 (2016).
4. J. Gao et al., Loss of IFN-γ pathway genes in tumor cells as a mechanism of resistance to anti-CTLA-4 therapy. Cell 167, 397-404 (2016).
5. J. M. Zaretsky et al., Mutations Associated with Acquired Resistance to PD-1 Blockade in Melanoma. N Engl J Med 375, 819-829 (2016).
6. J. M. Pitt et al., Resistance Mechanisms to Immune-Checkpoint Blockade in Cancer: Tumor-Intrinsic and -Extrinsic Factors. Immunity 44, 1255-1269 (2016).
7. A. Snyder et al., Genetic basis for clinical response to CTLA-4 blockade in melanoma. N Engl J Med 371, 2189-2199 (2014).
8. N. A. Rizvi et al., Cancer immunology. Mutational landscape determines sensitivity to PD-1 blockade in non-small cell lung cancer. Science 348, 124-128 (2015).
9. M. D. Hellmann et al., Nivolumab plus Ipilimumab in Lung Cancer with a High Tumor Mutational Burden. N Engl J Med 378, 2093-2104 (2018).
10. M. D. Hellmann et al., Tumor Mutational Burden and Efficacy of Nivolumab Monotherapy and in Combination with Ipilimumab in Small-Cell Lung Cancer. Cancer Cell 33, 853-861 (2018).
11. C. H. June, R. S. O'Connor, O. U. Kawalekar, S. Ghassemi, M. C. Milone, CAR T cell immunotherapy for human cancer. Science 359, 1361-1365 (2018).
12. E. O. Long, H. S. Kim, D. Liu, M. E. Peterson, S. Rajagopalan, Controlling natural killer cell responses: integration of signals for activation and inhibition. Annu Rev Immunol 31, 227-258 (2013).
13. D. H. Raulet, S. Gasser, B. G. Gowen, W. Deng, H. Jung, Regulation of ligands for the NKG2D activating receptor. Annu Rev Immunol 31, 413-441 (2013).
14. S. Bauer et al., Activation of NK cells and T cells by NKG2D, a receptor for stress-inducible MICA. Science 285, 727-729 (1999).
15. H. R. Salih, H. G. Rammensee, A. Steinle, Cutting edge: down-regulation of MICA on human tumors by proteolytic shedding. J Immunol 169, 4098-4102 (2002).
16. B. K. Kaiser et al., Disulphide-isomerase-enabled shedding of tumour-associated NKG2D ligands. Nature 447, 482-486 (2007).
17. M. Jinushi et al., MHC class I chain-related protein A antibodies and shedding are associated with the progression of multiple myeloma. Proc Natl Acad Sci USA 105, 1285-1290 (2008).
18. F. Q. Yang et al., Matrix metallopeptidase 2 (MMP2) mediates MHC class I polypeptide-related sequence A (MICA) shedding in renal cell carcinoma. Actas Urol Esp 38, 172-178 (2014).
19. P. Boutet et al., Cutting edge: the metalloproteinase ADAM17/TNF-alpha-converting enzyme regulates proteolytic shedding of the MHC class I-related chain B protein. J Immunol 182, 49-53 (2009).
20. V. Groh, J. Wu, C. Yee, T. Spies, Tumour-derived soluble MIC ligands impair expression of NKG2D and T-cell activation. Nature 419, 734-738 (2002).
21. I. Waldhauer et al., Tumor-associated MICA is shed by ADAM proteases. Cancer Res 68, 6368-6376 (2008).
22. H. R. Salih, D. Goehlsdorf, A. Steinle, Release of MICB molecules by tumor cells: mechanism and soluble MICB in sera of cancer patients. Hum Immunol 67, 188-195 (2006).
23. L. F. de Andrade et al., Antibody-mediated inhibition of MICA and MICB shedding promotes NK cell-driven tumor immunity. Science 359, 1537-1542 (2018).
24. P. Li et al., Complex structure of the activating immunoreceptor NKG2D and its MHC class I-like ligand MICA. Nat Immunol 2, 443-451 (2001).
25. S. Bahram, M. Bresnahan, D. E. Geraghty, T. Spies, A second lineage of mammalian major histocompatibility complex class I genes. Proc Natl Acad Sci USA 91, 6259-6263 (1994).
26. V. Groh et al., Cell stress-regulated human major histocompatibility complex class I gene expressed in gastrointestinal epithelium. Proc Natl Acad Sci USA 93, 12445-12450 (1996).
27. S. Armeanu et al., Natural killer cell-mediated lysis of hepatoma cells via specific induction of NKG2D ligands by the histone deacetylase inhibitor sodium valproate. Cancer Res 65, 6321-6329 (2005).
28. S. Skov et al., Cancer cells become susceptible to natural killer cell killing after exposure to histone deacetylase inhibitors due to glycogen synthase kinase-3-dependent expression of MHC class I-related chain A and B. Cancer Res 65, 11136-11145 (2005).
29. C. Zhang, Y. Wang, Z. Zhou, J. Zhang, Z. Tian, Sodium butyrate upregulates expression of NKG2D ligand MICA/B in HeLa and HepG2 cell lines and increases their susceptibility to NK lysis. Cancer Immunol Immunother 58, 1275-1285 (2009).
30. S. Diermayr et al., NKG2D ligand expression in AML increases in response to HDAC inhibitor valproic acid and contributes to allorecognition by NK-cell lines with single KIR-HLA class I specificities. Blood 111, 1428-1436 (2008).
31. B. Izar et al., Bidirectional cross talk between patient-derived melanoma and cancer-associated fibroblasts promotes invasion and proliferation. Pigment Cell & Melanoma Research 29, 656-668 (2016).
32. D. Pan et al., A major chromatin regulator determines resistance of tumor cells to T cell-mediated killing. Science 359, 770-775 (2018).
33. L. L. Chan, K. W. Wucherpfennig, L. F. de Andrade, Visualization and quantification of NK cell-mediated cytotoxicity over extended time periods by image cytometry. J Immunol Methods 469, 47-51 (2019).
34. L. Ferrari de Andrade et al., Natural killer cells are essential for the ability of BRAF inhibitors to control BRAFV600E-mutant metastatic melanoma. Cancer Res 74, 7298-7308 (2014).
35. S. Kim et al., Licensing of natural killer cells by host major histocompatibility complex class I molecules. Nature 436, 709-713 (2005).
36. Y. Y. Yu et al., The role of Ly49A and 5E6(Ly49C) molecules in hybrid resistance mediated by murine natural killer cells against normal T cell blasts. Immunity 4, 67-76 (1996).
37. L. Ruggeri et al., Effectiveness of donor natural killer cell alloreactivity in mismatched hematopoietic transplants. Science 295, 2097-2100 (2002).
38. X. Wang et al., A herpesvirus encoded Qa-1 mimic inhibits natural killer cell cytotoxicity through CD94/NKG2A receptor engagement. Elife 7, e38667 (2018).
39. J. P. Laubach, P. Moreau, J. F. San-Miguel, P. G. Richardson, Panobinostat for the Treatment of Multiple Myeloma. Clin Cancer Res 21, 4767-4773 (2015).
40. A. C. West et al., An Intact Immune System Is Required for the Anticancer Activities of Histone Deacetylase Inhibitors. Cancer Res 73, 7265-7276 (2013).
41. J. D. Growney et al. Blood 110, 1510-1510 (2007).
42. V. Groh, A. Steinle, S. Bauer, T. Spies, Recognition of stress-induced MHC molecules by intestinal epithelial gammadelta T cells. Science 279, 1737-1740 (1998).
43. L. Chiossone, P.-Y. Dumas, M. Vienne, E. Vivier, Natural killer cells and other innate lymphoid cells in cancer. Nature reviews Immunology 18, 671-688 (2018).
44. C. S. N. Klose et al., Differentiation of type 1 ILCs from a common progenitor to all helper-like innate lymphoid cell lineages. Cell 157, 340-356 (2014).
45. W. Deng et al., A shed NKG2D ligand that promotes natural killer cell activation and tumor rejection. Science 348, 136-139 (2015).
46. P. Dhar, J. D. Wu, NKG2D and its ligands in cancer. Curr Opin Immunol 51, 55-61 (2018).
47. S. C. Formenti et al., Radiotherapy induces responses of lung cancer to CTLA-4 blockade. Nat Med 24, 1845-1851 (2018).
48. R. Romee et al., First-in-human phase 1 clinical study of the IL-15 superagonist complex ALT-803 to treat relapse after transplantation. Blood 131, 2515-2527 (2018).
49. P. André et al., Anti-NKG2A mAb is a checkpoint inhibitor that promotes anti-tumor immunity by unleashing both T and NK cells. Cell 175, 1731-1743 (2018).

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of treating cancer in a subject, the method comprising:
administering to the subject a therapeutically effective amount of a composition comprising one or more activating agent(s) and a pharmaceutically acceptable carrier, wherein the activating agent(s) activates NK cells through the NKG2D and/or CD16 receptors, thereby causing lysis of one or more cancer cells in the subject, and wherein the cancer is resistant to cytotoxic T cells.

2. The method of claim 1, wherein the activating agent comprises a polynucleotide, a polypeptide, a small molecule, or a combination thereof.

3. The method of claim 1, wherein the activating agent comprises an anti-MICA antibody, an anti-MICB antibody, or both.

4. The method of claim 3, wherein the antibody comprises a monoclonal antibody.

5. The method of claim 3, wherein the antibody binds the alpha-3 domain of MICA/B.

6. The method of claim 3, wherein the antibody comprises one or more sequences of Table 1.

7. The method of claim 1, wherein the activating agent(s) inhibits MICA/MICB shedding by the tumor, thereby increasing the density of NKG2D receptor ligands on tumor cells.

8. The method of claim 1, further comprising administering to the subject a therapeutically effective amount of a second composition comprising one or more therapeutic agent(s) and a pharmaceutically acceptable carrier.

9. The method of claim 8, wherein the one or more therapeutic agent(s) comprises a small molecule, a toxin, a radiolabel, radiotherapy, an siRNA, a peptide, an antibody, a genetically engineered cell, radiation, or a cytokine.

10. The method of claim 8, wherein the one or more therapeutic agent comprises an HDAC inhibitor.

11. The method of claim 10, wherein the HDAC inhibitor is panobinostat.

12. The method of claim 9, wherein the cytokine comprises IL2, IL15, IL12, or IL18.

13. The method of claim 9, wherein the small molecule comprises a proteasome inhibitor.

14. The method of claim 9, wherein the antibody comprises an anti-PD1 antibody and/or anti-CTLA-4 antibody.

15. The method of claim 9, wherein the genetically engineered cell is a CAR T cell.

16. The method of claim 1, wherein the cancer is an MCH class I deficient cancer or a cancer resistant to IFN gamma.

17. The method of claim 1, wherein the cancer is resistant to immunotherapy.

18. The method of claim 1, wherein the cancer is resistant to anti-PD1/PD-L1 antibodies.

19. The method of claim 1, wherein the cancer comprises melanoma, lung cancer, renal cancer, bladder cancer, Hodgkin's lymphoma, breast cancer, stomach cancer, and pancreatic cancer.

20. The method of claim 1, wherein treating cancer is indicated by stopping or reducing tumor growth and/or metastasis.

21. The method of claim 1, further comprising a step of testing the cancer for a Jak1 mutation and/or a B2m mutation.

22. A method of sensitizing a cancer cell in a subject to NK cells, the method comprising:
administering to the subject a therapeutically effective amount of a composition comprising one or more activating agent(s) and a pharmaceutically acceptable carrier, wherein the activating agent(s) activates NK cells, thereby causing lysis of one or more cancer cells in the subject, and wherein the cancer is resistant to cytotoxic T cells.

23. The method of claim 22, wherein activation of the NKG2D receptor and/or CD16 receptor activates NK cells.

24. The method of claim 22, wherein the activating agent(s) inhibits MICA/MICB shedding by the cancer cell, thereby activating the NKG2D and/or CD16 receptor.

25. The method of claim 22, wherein MICA/B on the surface of the cancer cell activates the NKG2D receptor, the CD16 receptor, or both.

26. The method of claim 22, wherein the cancer is an MCH class I deficient cancer or a cancer resistant to IFN gamma.

27. The method of claim 22, further comprising a step of testing the cancer cell for a Jak1 mutation and/or a B2m mutation.
